(19) United States
(12) Patent Application Publication
(10) Pub. No.: US 2003/0162223 A1
(43) Pub. Date: Aug. 28, 2003

(54) Drosophila G protein coupled receptors, nucleic acids, and methods related to the same

(76) Inventors: David E. Lowery, Kalamazoo, MI (US); Valdin G. Smith, Kalamazoo, MI (US); Teresa M. Kubiak, Richland, MI (US); Martha J. Larsen, Kalamazoo, MI (US)

Correspondence Address: COZEN O ' CONNER, P.C., 1900 MARKET STREET, PHILADELPHIA, PA, 19103-3508, US

(21) Appl. No.: 10/283,423

(22) Filed: Oct. 30, 2002

Related U.S. Application Data
(63) Continuation-in-part of application No. 10/213,821, filed on Aug. 6, 2002.
Application No. 10/213,821 is a continuation-in-part of application No. 09/693,746, filed on Oct. 20, 2000.
Application No. 09/693,746 is a continuation-in-part of application No. 09/425,676, filed on Oct. 22, 1999, now abandoned.

(51) Int. Cl. G01N 33/53; A61K 39/395; A01N 25/00; A01N 63/00

(52) U.S. Cl. 435/7.1; 424/405; 514/12; 424/143.1

(57) ABSTRACT

The present invention provides Drosophila melanogaster GPCR (DmGPCR) polypeptides and polynucleotides which identify and encode such a DmGPCR. In addition, the invention provides expression vectors, host cells, and methods for its production. The invention also provides methods for the identification of homologs in other species and of DmGPCR agonists/antagonists useful as potential insecticides. The invention further provides methods for binding a DmGPCR, methods for identifying modulators of DmGPCR expression and activity, methods for controlling a population of insects with a DmGPCR antibody, a DmGPCR antisense polynucleotide, a DmGPCR binding partner or modulator, and methods of preventing or treating a disease or condition associated with an ectoparasite. Specifically, this invention discloses the matching of the orphan Drosophila short neuropeptide F receptor with its cognate peptide ligands.

### CROSS REFERENCE TO RELATED APPLICATIONS

This application is a continuation-in-part of U.S. patent application Ser. No. 10/213,821, filed Aug. 6, 2002, which is a continuation-in-part of U.S. patent application Ser. No. 09/693,746, filed Oct. 20, 2000, which is a continuation-in-part of U.S. patent application Ser. No. 09/425,676, filed Oct. 22, 1999, each of which is hereby incorporated by reference in its entirety.

### FIELD OF THE INVENTION

The present invention is directed, in part, to nucleic acid molecules encoding novel Drosophila melanogaster G protein coupled receptors (DmGPCRs), novel polypeptides, assays for screening compounds that bind to a DmGPCR and/or modulate the activity of a DmGPCR, methods for binding a DmGPCR, reagents such as antibodies to a DmGPCR, primers, and probes for detection of nucleotide sequences encoding a DmGPCR, kits including the antibodies, primers, and probes of the invention, compositions including DmGPCRs, DmGPCR binding partners, and DmGPCR modulators, and methods for controlling an insect population using a DmGPCR binding partner or modulator.

### BACKGROUND OF THE INVENTION

Humans and other life forms are comprised of living cells. Among the mechanisms through which the cells of an organism communicate with each other and obtain information and stimuli from their environment is cell membrane receptor molecules expressed on the cell surface. Many such receptors have been identified, characterized, and sometimes classified into major receptor superfamilies based on structural motifs and signal transduction features. Such families include (but are not limited to) ligand-gated ion channel receptors, voltage-dependent ion channel receptors, receptor tyrosine kinases, receptor protein tyrosine phosphatases, and G protein-coupled receptors. The receptors are a first essential link for translating an extracellular signal into a cellular physiological response.

G protein-coupled receptors (i.e., GPCRs) form a vast superfamily of cell surface receptors which are characterized by an amino-terminal extracellular domain, a carboxy-terminal intracellular domain, and a serpentine structure that passes through the cell membrane seven times. Hence, such receptors are sometimes also referred to as seven transmembrane (7TM) receptors. These seven transmembrane domains define three extracellular loops and three intracellular loops, in addition to the amino- and carboxy-terminal domains. The extracellular portions of the receptor have a role in recognizing and binding one or more extracellular binding partners (e.g., ligands), whereas the intracellular portions have a role in recognizing and communicating with downstream effector molecules.

The GPCRs bind a variety of ligands including calcium ions, hormones, chemokines, neuropeptides, neurotransmitters, nucleotides, lipids, odorants, and even photons. Not surprisingly, GPCRs are important in the normal (and sometimes the aberrant) function of many cell types. See generally Strosberg, Eur. J. Biochem., 1991, 196, 1-10; Bohm et al., Biochem J., 1997, 322, 1-18. When a specific ligand binds to its corresponding receptor, the ligand typically stimulates the receptor to activate a specific heterotrimeric guanine nucleotide-binding regulatory protein (G protein) that is coupled to the intracellular portion or region of the receptor. The G protein, in turn, transmits a signal to an effector molecule within the cell by either stimulating or inhibiting the activity of that effector molecule. These effector molecules include adenylate cyclase, phospholipases, and ion channels. Adenylate cyclase and phospholipases are enzymes that are involved in the production of the second messenger molecules cAMP, inositol triphosphate, and diacyglycerol. It is through this sequence of events that an extracellular ligand stimulus exerts intracellular changes through a G protein-coupled receptor. Each such receptor has its own characteristic primary structure, expression pattern, ligand binding profile, and intracellular effector system.

Because of the vital role of G protein-coupled receptors in the communication between cells and their environment, such receptors are attractive targets for regulation, for example, by activating or antagonizing such receptors. For receptors having a known ligand, the identification of agonists or antagonists may be sought specifically to enhance or inhibit the action of the ligand. For example, some G protein-coupled receptors have roles in disease pathogenesis (e.g., certain chemokine receptors that act as HIV co-receptors may have a role in AIDS pathogenesis), and are attractive targets for therapeutic intervention even in the absence of knowledge of the natural ligand of the receptor. Other receptors are attractive targets for therapeutic intervention by virtue of their expression pattern in tissues or cell types that are themselves attractive targets for therapeutic intervention. Examples of this latter category of receptors include receptors expressed in immune cells, which can be targeted to either inhibit autoimmune responses or to enhance immune responses to fight pathogens or cancer; and receptors expressed in the brain or other neural organs and tissues, which are likely targets in the treatment of schizophrenia, depression, bipolar disease, or other neurological disorders. This latter category of receptor is also useful as a marker for identifying and/or purifying (e.g., via fluorescence-activated cell sorting) cellular subtypes that express the receptor.

Insects are recognized as major pests in agriculture and in human domestic environments. Insects also parasitize animals and humans, being denoted as ectoparasites in such cases, causing morbidity and mortality. Insects also serve as vectors for the transmission of viral and parasitic diseases to plants, animals and humans. Thus, there is a continuing and compelling need to discover new methods for controlling insect populations and for repelling and/or killing pathogenic or pestiferous species. One way to control insect populations by killing or paralyzing insects is through the use of chemical agents, denoted as insecticides, that are selectively toxic to insects and potentially other invertebrates. Currently, insecticides have enormous value for the control of insects that are damaging to agricultural products, including crops and livestock. Insecticides are also used in human domestic situations, for the control of lawn and garden pests as well as insects that are damaging or annoying to humans, including stinging or biting insects, flies and cockroaches. Insecticides also have enormous value for the treatment or prevention of disease states caused by ectoparasites, including fleas, lice, ticks, mites, and biting flies, in livestock animals and pets. However, current chemicals used as insecticide are not optimal. Some have demonstrable toxicity for mammals, while resistance to some of them has arisen in certain target species. Therefore, there exists a need for new selective insecticides that have novel mechanisms of action.

Examples of insect GPCRs that have neuropeptide ligands are known (see, e.g., Li, et al., EMBO Journal, 1991, 10, 3221-3229; Li, et al., J. Biol. Chem., 1992, 267, 9-12; Monnier, et al., J. Biol. Chem., 1992, 267, 1298-1302; Vanden Broeck, et al., Int. Rev. Cytology, 1996, 164, 189-268; Guerrero, Peptides, 1997, 18, 1-5; Hauser, et al., J. Biol. Chem., 1997, 272, 1002-1010; Birgul et al., EMBO J, 1999, 18, 5892-5900; Torfs et al., J. Neurochem., 2000, 74, 2182-2189; and Hauser et al., Biochem. Biophys. Res. Comm., 1998, 249, 822-828; Larsen, et al., Biochem. Biophys. Res. Comm., 2001, 286, 895-901; Lenz, et al., Biochem. Biophys. Res. Comm., 2001, 286, 1117-1122; Kubiak et al., Biochem. Biophys. Res. Comm., 2002, 291, 313-320; Staubli et al., Proc. Natl. Acad. Sci. USA, 2002, 99, 3446-3451; Garczynski et al., Peptides, 2002,23, 773-780), Holmes et al. Insect Molecular Biology, 2000, (5), 457-465; Cazzamali et al. Proc. Natl. Acad. Sci. USA, 2002, 99, 12073-12078; Cazzamali et al., Biochem. Biophys. Res. Comm., 2002, 298, 31-36; Radford et. al., J. Biol. Chem. 2002, 277, 38810-38817; Park et al., Proc. Natl. Acad. Sci. USA, 2002, 99, 11423-11428; Kreienkamp et al., J. Biol. Chem, 10.1074/jbc.M206931200 (published online Aug. 6, 2002) and Mertens, et al., Biochem. Biophys. Res. Comm., 2002, 297, 1140-1148. Recent related patent applications: Ebens, Allen James, Jr.; Torpey, Justin; Keegan, Kevin Patrick. Nucleic acids and polypeptides of Drosophila melanogaster G protein-coupled receptor and their use as pesticidal and pharmaceutical targets. PCT Int. Appl. (2001), 43 pp. CODEN: PIXXD2 WO 0170981 A220010927 CAN 135:268323 AN 2001:713564 CAPLUS. Kravchik, Anibal. Drosophila G protein-coupled receptors, genomic DNA and cDNA molecules encoding GPCR proteins, and their uses as insecticidal targets. PCT Int. Appl. (2001), 392 pp. CODEN: PIXXD2 WO 0170980 A220010927 CAN 135:269068 AN

A large family of peptides generally 4-12 amino acids in length typically found in invertebrate animals (e.g., insects) is a class of neuropeptides known as FMRFamide-related peptides (i.e., FaRPs). The prototypical FMRFamide (FMRFa) peptides are so named because of the “FMRF” consensus amino acid sequence at their C-termini, consisting generally of (F,Y)(M,V,I,L)R(F,Y)NH₂. As neuropeptides, these molecules are involved in vital biological processes requiring controlled neuromuscular activity. Although some neurotransmitters and neuromodulators (including neuropeptides) have been shown to function as ligands for receptors, to date there has been no identification of a FaRP neuropeptide as a ligand of a GPCR.

Drosophila peptides containing a conserved FXGXR-amide motif are structurally related to mammalian tachykinins and, hence, have been coined drotachykinins (Siviter et al., J. Biol. Chem., 2000, 275(30), 23273-23280). The drotachykinins have potent stimulatory effects on contractions of the insect gut (id.).

Leucokinins are a group of widespread insect hormones that stimulate gut motility and tubule fluid secretion rates. In tubules, their major action is to raise chloride permeability by binding to a receptor on the basolateral membrane. Leucokinin acts by raising intracellular calcium in only the stellate cells (O'Donnell et al., Am. J. Physiol., 1998, 43, R1039-R1049).

The allatostatins are an important group of insect neurohormones controlling diverse functions including the synthesis of juvenile hormones known to play a central role in metamorphosis and reproduction in various insect species. The very first Drosophila allatostatin, Ser-Arg-Pro-Tyr-Ser-Phe-Gly-Leu-NH₂ (i.e., drostatin-3) (SEQ ID NO: 165), was isolated from Drosophila head extracts (Birgul et al., EMBO J., 1999, 18, 5892-5900). Recently, a Drosophila allatostatin preprophormone gene has been cloned which encodes four Drosophila allatostatins: Val-Glu-Arg-Tyr-Ala-Phe-Gly-Leu-NH₂ (drostatin-1) (SEQ ID NO: 163), Leu-Pro-Val-Tyr-Asn-Phe-Gly-Leu-NH₂ (drostatin-2) (SEQ ID NO: 164), Ser-Arg-Pro-Tyr-Ser-Phe-Gly-Leu-NH₂ (drostatin-3) (SEQ ID NO: 165), and Thr-Thr-Arg-Pro-Gln-Pro-Phe-Asn-Phe-Gly-Leu-NH₂ (drostatin-4) (SEQ ID NO: 166) (Lenz et al., Biochem. Biophys. Res. Comm., 2000, 273, 1126-1131). The first Drosophila allatostatin receptor was cloned by Birgul et al. and was shown to be functionally activated by drostatin-3 via Gi/Go pathways (Birgul et al., EMBO J. 1999, 18, 5892-5900). A second putative Drosophila allatostatin receptor (i.e., DAR11) has been recently cloned (Lenz et al., Biochem. Biophys. Res. Comm., 2000, 273, 571-577). The DAR11 receptor cDNA (Accession No. AF253526) codes for a protein that is strongly related to the first Drosophila allatostatin receptor. Recently, functional activation of DAR11 by allatostatins have been shown by us (Larsen, et al., Biochem. Biophys. Res. Comm., 2001, 286, 895-901) and others (Lenz, et al., Biochem. Biophys. Res. Comm., 2001, 286, 1117-1122). Recently, a Drosophila allatostatin type C preprophormone gene has been cloned which encodes a Drosophila allatostatin-C: Gln-Val-Arg-Tyr-Gln-Cys-Tyr-Phe-Asn-Pro-Ile-Ser-Cys-Phe-OH (Williamson et al., Biochem. Biophys. Res. Comm., 2001, 282, 124-130). The mature peptide should have a pGlu at the N-terminus, formed as a result of the N-terminal Gln cyclization, to yield: pGlu-Val-Arg-Tyr-Gln-Cys-Tyr-Phe-Asn-Pro-Ile-Ser-Cys-Phe-OH (SEQ ID NO: 183), and a disulfide bridge between Cys⁶ and Cys¹³, similar to the Manduca sexta type C allatostatin, pGlu-Val-Arg-Phe-Gln-Cys-Tyr-Phe-Asn-Pro-Ile-Ser-Cys-Phe-OH (SEQ ID NO: 182)., which differs only at position 4 (Phe⁴ vs Tyr⁴) (Kramer et al., Proc. Natl. Acad. Sci. USA, 1991, 88, 9458-9462). Nichols at al., showed potent and prolonged inhibition of muscle contraction of the Drosophila allatostatin-C and named it a flatline (FLT) peptide (Nichols et al. Peptides, 2002, 23, 787-794). To our knowledge, to date no receptors for insect allatostatin type-C have been identified.

The sulfakinins are a family of insect Tyr-sulfated neuropeptides. They show sequence and functional (myotropic effects, stimulation of digestive enzyme release) similarity to the vertebrate peptides gastrin and cholecystokinin. A gene encoding two sulfakinins (also called drosulfakinins), DSKI [Phe-Asp-Asp-Tyr(SO₃H)-Gly-His-Met-Arg-Phe-amide] (SEQ ID NO: 160) and DSKII [Gly-Gly-Asp-Asp-Gln-Phe-Asp-Asp-Tyr(SO₃H)-Gly-His-Met-Arg-Phe-amide] (SEQ ID NO: 161), has been identified in Drosophila melanogaster (Nichols, Mol. Cell Neuroscience, 1992, 3, 342-347; Nichols et al., J. Biol. Chem., 1988, 263, 12167-12170). The C-terminal heptapeptide sequence, Asp-Tyr(SO₃H)-Gly-His-Met-Arg-Phe-amide (SEQ ID NO: 162), is identical in all sulfakinins identified so far from insects that are widely separated in evolutionary terms. The conservation of the heptapeptide sequence, including the presence of the sulfated Tyr residue, in widely divergent insect taxa presumably reflects functional significance of this myotropic “active core” (Nachman & Holman, in INSECT NEUROPEPTIDES: CHEMISTRY, BIOLOGY AND ACTION, Menn, Kelly & Massler, Eds., American Chemical Society, Washington, D.C., 1991, pp. 194-214). Recently, we identified the Drosophila orphan receptor (DmGPCR9) as a drosulfakinin receptor (named DSK-R1) and matched it with its activating peptide, a Met⁵→Leu modified drosulfakinin-1, Asp-Tyr(SO₃H)-Gly-His-Leu-Arg-Phe-amide (SEQ ID NO: 157) (Kubiak et al., Biochem. Biophys. Res. Comm., 2002, 291, 313-320). The new de-orphaned Drosophila GPCRs include recptors for PRXamide peptides, CCAP, corazonin, and AKH (Park et al., Proc. Natl. Acad. Sci. USA, 2002, 99, 11423-11428; Cazzamali et al., Biochem. Biophys. Res. Comm., 2002, 298, 31-36); leukokinin (Radford et. al., J. Biol. Chem. 2002, 277, 38810-38817); Drostatin-C (Kreienkamp et al., J. Biol. Chem, 10.1074/jbc.M206931200 (published online Aug. 6, 2002)); FMRFamide (Cazzamali et al. Proc. Natl. Acad. Sci. USA, 2002, 99, 12073-12078); and neuoropeptide F (Mertens, et al., Biochem. Biophys. Res. Comm., 2002, 297, 1140-1148).

### SUMMARY OF THE INVENTION

The present invention involves the surprising discovery of novel polypeptides in Drosophila melanogaster, designated herein DmGPCRs (Drosophila melanogaster G Protein-Coupled Receptors), which exhibit varying degrees of homology to other neuropeptide GPCRs. The present invention provides genes encoding these heretofore unknown G protein-coupled receptors, the DmGPCR polypeptides encoded by the genes; antibodies to the polypeptides; kits employing the polynucleotides and polypeptides, and methods of making and using all of the foregoing. The DmGPCRs may play a role as a key component, for example, in regulating neuropeptide binding and/or signaling. DmGPCRs are thus useful in the search for novel agents that can modify and/or control binding and/or signaling by neuropeptides or other agents. These and other aspects of the invention are described below.

In some embodiments, the invention provides purified and isolated DmGPCR polypeptides comprising the amino acid sequence set forth in any of SEQ ID NOs: 2, 4, 6, 8, 10, 12, 14, 16, 18, 20, 22, or 24, or a fragment thereof comprising an epitope specific to the DmGPCR. By “epitope specific to” is meant a portion of the DmGPCR receptor that is recognizable by an antibody that is specific for the DmGPCR, as defined in detail below. One embodiment of the invention comprises purified and isolated polypeptides comprising the complete amino acid sequences set forth in SEQ ID NOs: 2, 4, 6, 8, 10, 12, 14, 16, 18, 20, 22, or 24, found in Table 4 below. These amino acid sequences were deduced from polynucleotide sequences encoding DmGPCR (SEQ ID NOs: 1, 3, 5, 7, 9, 11, 13, 15, 17, 19, 21, or 23, found in Table 4 below). The term “DmGPCR” as used herein in singular form is intended to encompass each of the ten amino acid sequences exemplified below, encoded by the respective polynucleotide sequences.

Although the sequences provided are particular Drosophila sequences, the invention is intended to include within its scope allelic variants, vertebrate, and invertebrate forms of DmGPCR.

In some embodiments, the invention provides purified and isolated polynucleotides (e.g., cDNA, genomic DNA, synthetic DNA, RNA, or combinations thereof, whether single- or double-stranded) that comprise a nucleotide sequence encoding the amino acid sequence of the polypeptides of the invention. Such polynucleotides are useful for recombinantly expressing the receptor and also for detecting expression of the receptor in cells (e.g., using Northern hybridization and in situ hybridization assays). Such polynucleotides also are useful in the design of antisense and other molecules for the suppression or regulation of the expression of DmGPCR in a cultured cell, a tissue, or an animal. Specifically excluded from the definition of polynucleotides of the invention are entire isolated, non-recombinant native chromosomes of host cells. Polynucleotides of the invention may have the sequence of any sequence set forth in SEQ ID NOs: 1, 3, 5, 7, 9, 11, 13, 15, 17, 19, 21, or 23, which correspond to naturally occurring DmGPCR sequences. It will be appreciated that numerous other polynucleotide sequences exist that also encode the DmGPCR having the sequence set forth in any of SEQ ID NOs: 2, 4, 6, 8, 10, 12, 14, 16, 18, 20, 22, or 24 due to the well-known degeneracy of the universal genetic code.

The invention also provides a purified and isolated polynucleotide comprising a nucleotide sequence that encodes a mammalian polypeptide, wherein the polynucleotide hybridizes to a polynucleotide having the sequence set forth in any of SEQ ID NOs: 1, 3, 5, 7, 9, 11, 13, 15, 17, 19, 21, or 23 or the non-coding strand complementary thereto, under the following hybridization conditions:

(a) hybridization for 16 hours at 42° C. in a hybridization solution comprising 50% formamide, 1% SDS, 1 M NaCl, 10% dextran sulfate; and

(b) washing 2 times for 30 minutes each at 60° C. in a wash solution comprising 0.1% SSC, 1% SDS.

Hybridization conditions should be such that hybridization occurs only with the genes in the presence of other nucleic acid molecules. Under stringent hybridization conditions only highly complementary nucleic acid sequences hybridize. Such conditions may prevent hybridization of nucleic acids having 1 or 2 mismatches out of 20 contiguous nucleotides.

In some embodiments, the invention provides vectors comprising a polynucleotide of the invention. Such vectors are useful, e.g., for amplifying the polynucleotides in host cells to create useful quantities thereof. In some embodiments, the vector is an expression vector wherein the polynucleotide of the invention is operatively linked to a polynucleotide comprising an expression control sequence. Such vectors are useful for recombinant production of polypeptides of the invention.

In some embodiments, the invention provides host cells that are transformed or transfected (stably or transiently) with polynucleotides of the invention or vectors of the invention. As stated above, such host cells are useful for amplifying the polynucleotides and also for expressing the DmGPCR polypeptide or fragment thereof encoded by the polynucleotide.

In still another embodiment, the invention provides methods for producing a DmGPCR polypeptide (or fragment thereof) comprising the steps of growing a host cell of the invention in a nutrient medium and isolating the polypeptide or variant thereof from the cell or the medium. Because DmGPCR is a seven transmembrane receptor, it will be appreciated that, for some applications, such as certain activity assays, the isolation may involve isolation of cell membranes containing the polypeptide embedded therein, whereas for other applications a more complete isolation may be desired.

It will be appreciated that extracellular epitopes are particularly useful for generating and screening for antibodies and other binding compounds that bind to receptors such as DmGPCR. Thus, in another embodiment, the invention provides a purified and isolated polypeptide comprising at least one extracellular domain (e.g., the N-terminal extracellular domain or one of the three extracellular loops) of DmGPCR such as the N-terminal extracellular domain of DmGPCR. Also included in the invention are purified polypeptides comprising transmembrane domains of DmGPCR, an extracellular loop connecting transmembrane domains of DmGPCR, an intracellular loop connecting transmembrane domains of DmGPCR, the C-terminal cytoplasmic region of DmGPCR, and fusions thereof. Such fragments may be continuous portions of the native receptor. However, it will also be appreciated that knowledge of the DmGPCR gene and protein sequences as provided herein permits recombining of various domains that are not contiguous in the native protein.

In still another embodiment, the invention provides antibodies specific for the DmGPCR of the invention. Antibody specificity is described in greater detail below. However, it should be emphasized that antibodies that can be generated from polypeptides that have previously been described in the literature and that are capable of fortuitously cross-reacting with DmGPCR (e.g., due to the fortuitous existence of a similar epitope in both polypeptides) are considered “cross-reactive” antibodies. Such cross-reactive antibodies are not antibodies that are “specific” for DmGPCR. The determination of whether an antibody is specific for DmGPCR or is cross-reactive with another known receptor is made using any of several assays, such as Western blotting assays, that are well-known in the art. For identifying cells that express DmGPCR and also for modulating DmGPCR-ligand binding activity, antibodies that specifically bind to an extracellular epitope of the DmGPCR may be used.

In one variation, the invention provides monoclonal antibodies. Hybridomas that produce such antibodies also are intended as aspects of the invention.

In another variation, the invention provides a cell-free composition comprising polyclonal antibodies, wherein at least one of the antibodies is an antibody of the invention specific for DmGPCR. Antisera isolated from an animal is an exemplary composition, as is a composition comprising an antibody fraction of an antisera that has been resuspended in water or in another diluent, excipient, or carrier.

In still another related embodiment, the invention provides anti-idiotypic antibodies specific for an antibody that is specific for DmGPCR.

It is well-known that antibodies contain relatively small antigen binding domains that can be isolated chemically or by recombinant techniques. Such domains are useful DmGPCR binding molecules themselves, and also may be fused to toxins or other polypeptides. Thus, in still another embodiment, the invention provides a polypeptide comprising a fragment of a DmGPCR-specific antibody, wherein the fragment and the polypeptide bind to the DmGPCR. By way of non-limiting example, the invention provides polypeptides that are single chain antibodies, CDR-grafted antibodies, and humanized antibodies.

Also within the scope of the invention are compositions comprising polypeptides, polynucleotides, or antibodies of the invention that have been formulated with, e.g., a pharmaceutically acceptable carrier.

The invention also provides methods of using antibodies of the invention. For example, the invention provides methods for modulating ligand binding of a DmGPCR comprising the step of contacting the DmGPCR with an antibody specific for the DmGPCR, under conditions wherein the antibody binds the receptor.

The invention provides methods of inducing an immune response in a subject against a polypeptide comprising a sequence from the group of SEQ ID NOs: 2, 4, 6, 8, 10, 12, 14, 16, 18, 20, 22, and 24, or a homolog or fragment thereof. The methods comprise administering to a subject an amount of the polypeptide sufficient to induce the immune response.

The invention also provides assays to identify compounds that bind a DmGPCR. One such assay comprises the steps of: (a) contacting a composition comprising a DmGPCR with a compound suspected of binding DmGPCR; and (b) measuring binding between the compound and DmGPCR. In one variation, the composition comprises a cell expressing DmGPCR on its surface. In another variation, isolated DmGPCR or cell membranes comprising DmGPCR are employed. The binding may be measured directly, e.g., by using a labeled compound, or may be measured indirectly by several techniques, including measuring intracellular signaling of DmGPCR induced by the compound (or measuring changes in the level of DmGPCR signaling).

The invention also provides methods of binding a DmGPCR with a binding partner. The methods comprise the steps of: (a) contacting a composition comprising a DmGPCR with a binding partner and (b) allowing the binding partner to bind the DmGPCR. For example, the DmGPCR may be DmGPCR1 (SEQ ID NO: 1), DmGPCR5 (SEQ ID NO: 9), DmGPCR7 (SEQ ID NO: 17), or DmGPCR8 (SEQ ID NO: 19). The binding partner may be, for example, a drotachykinin, a leucokinin, or an allatostatin-C. The drotachykinin (DTK) may be, for example, DTK-1 (SEQ ID NO: 169), Met8-DTK-2 (SEQ ID NO: 170), DTK-2 (SEQ ID NO: 171), DTK-3 (SEQ ID NO: 172), DTK-4 (SEQ ID NO: 173), and DTK-5 (SEQ ID NO: 174). The leucokinin (LK) may be, for example, LK-I (SEQ ID NO: 175), LK-V (SEQ ID NO: 176), LK-VI (SEQ ID NO: 177), and LK-VIII (SEQ ID NO: 178), Culekinin (SEQ ID NO: 179), mollusc leucokinin-like peptide, lymnokinin (PSFHSWSa) (SEQ ID NO: 180), and Drosophila leucokinin-like peptides DLK-1 (NSVVLGKKQRFHSWGa) (SEQ ID NO: 181), DLK-2 (pGlu-RFHSWGa) (SEQ ID NO: 182) and DLK-2A (QRFHSWGa) (SEQ ID NO: 183). The allatostatin (AST) may be, for example, AST-C (SEQ ID NO: 184), or DST-C (SEQ ID NO: 185). Other binding partners include, without limitation, SEQ ID NO: 186 and SEQ ID NO: 187.

The invention also provides methods for identifying a modulator of binding between a DmGPCR and a DmGPCR binding partner, comprising the steps of: (a) contacting a DmGPCR binding partner and a composition comprising a DmGPCR in the presence and in the absence of a putative modulator compound; (b) detecting binding between the binding partner and the DmGPCR; and (c) identifying a putative modulator compound or a modulator compound in view of decreased or increased binding between the binding partner and the DmGPCR in the presence of the putative modulator, as compared to binding in the absence of the putative modulator. For example, the DmGPCR may be DmGPCR5 (SEQ ID NO: 9), DmGPCR7 (SEQ ID NO: 17), or DmGPCR8 (SEQ ID NO: 19). The binding partner may be, for example, a drotachykinin, a leucokinin, or an allatostatin. The drotachykinin (DTK) may be, for example, DTK-1 (SEQ ID NO: 169), Met8-DTK-2 (SEQ ID NO: 170), DTK-2 (SEQ ID NO: 171), DTK-3 (SEQ ID NO: 172), DTK-4 (SEQ ID NO: 173), and DTK-5 (SEQ ID NO: 174). The leucokinin (LK) may be, for example, LK-I (SEQ ID NO: 175), LK-V (SEQ ID NO: 176), LK-VI (SEQ ID NO: 177), and LK-VIII (SEQ ID NO: 178), Culekinin (SEQ ID NO: 179), mollusc leucokinin-like peptide, lymnokinin (PSFHSWSa) (SEQ ID NO: 180), and Drosophila leucokinin-like peptides DLK-1 (NSVVLGKKQRFHSWGa) (SEQ ID NO: 181), DLK-2 (pGlu-RFHSWGa) (SEQ ID NO: 182), and DLK-2A (QRFHSWGa) (SEQ ID NO: 183). The allatostatin (AST) may be, for example, AST-C (SEQ ID NO: 184), or DST-C (SEQ ID NO: 185). In one variation, the composition comprises a cell expressing DmGPCR on its surface. In another variation, isolated DmGPCR or cell membranes comprising DmGPCR are employed. The binding may be measured directly, e.g., by using a labeled compound, or may be measured indirectly by several techniques, including measuring intracellular signaling of DmGPCR induced by the compound (or measuring changes in the level of DmGPCR signaling). For example, the function may be measured by an agonist induced [³⁵S]GTPγS binding assay, by cAMP assay (induction or inhibition of cAMP production), or by measuring intracellular calcium levels using fluorometric imaging plate reader (FLIPR) analysis.

DmGPCR binding partners that stimulate DmGPCR activity are useful as agonists to enhance or prolong DmGPCR signaling and this way to interfere with normally activated receptor signaling pathways. DmGPCR binding partners that block ligand-mediated DmGPCR signaling are useful as DmGPCR antagonists to to interfere with normal DmGPCR signaling and impair receptor-mediated effects. In addition, DmGPCR modulators, as well as DmGPCR polynucleotides and polypeptides, are useful in diagnostic assays for states or conditions in which DmGPCR activity is enhanced or impaired.

In another aspect, the invention provides methods for treating a disease or abnormal condition caused by an ectoparasite by administering to a subject in need of such treatment a substance that modulates the activity or expression of a polypeptide of the ectoparasite selected from the group consisting of SEQ ID NOs: 2, 4, 6, 8, 10, 12, 14, 16, 18, 20, 22, or 24.

Substances useful for treatment of disorders or diseases caused by an ectoparasite may show positive results in one or more in vitro assays for an activity corresponding to treatment of the disease or disorder in question. Substances that modulate the activity of the polypeptides include, but are not limited to, antisense oligonucleotides, agonists and antagonists, and antibodies.

In another aspect, the invention features methods for detection of a polypeptide in a sample as a diagnostic tool for diseases or disorders caused by an ectoparasite, wherein the methods comprise the steps of: (a) contacting the sample with a nucleic acid probe which hybridizes under hybridization assay conditions to a nucleic acid target region encoding a polypeptide selected from the group consisting of SEQ ID NOs: 2, 4, 6, 8, 10, 12, 14, 16, 18, 20, 22, or 24, said probe comprising the nucleic acid sequence encoding the polypeptide, fragments thereof, and/or the complements of the sequences and fragments; and (b) detecting the presence or amount of the probe:target region hybrid as an indication of the condition.

The test samples suitable for nucleic acid probing methods of the present invention include, for example, cells or nucleic acid extracts of cells, or biological fluids. The samples used in the above-described methods will vary based on the assay format, the detection method and the nature of the tissues, cells or extracts to be assayed. Methods for preparing nucleic acid extracts of cells are well-known in the art and can be readily adapted in order to obtain a sample that is compatible with the method utilized.

In some embodiments the present invention provides homologs, such as mammalian homologs, of DmGCPRs. Mammalian homologs of DmGPCR may be expressed in tissues including but not limited to tissues of the nervous system, pancreas (and particularly pancreatic islet tissue), pituitary, skeletal muscle, adipose tissue, liver, gastrointestinal (GI)-tract, and thyroid.

In some embodiments, the present invention provides methods of identifying a mammalian homolog of DmGPCR comprising the steps of screening a nucleic acid database or a nucleic acid library of the mammal with a nucleic acid molecule selected from SEQ ID NOs: 1, 3, 5, 7, 9, 11, 13, 15, 17, 19, 21, and 23, or a portion thereof, and determining whether a portion of the database or library is homologous to the sequence.

Another aspect of the invention provides methods of controlling an insect population by administering a binding partner or a modulator of a DmGPCR polynucleotide or polypeptide to an insect to modify the expression or activity of the DmGPCR. For example, the insect may be selected from the group consisting of a fly, a fruitfly, a tick, a flea, lice, a mite, and a cockroach.

The DmGPCR binding partner may be a drotachykinin (e.g., DTK-1 (SEQ ID NO: 169), Met8-DTK-2 (SEQ ID NO: 170), DTK-2 (SEQ ID NO: 171), DTK-3 (SEQ ID NO: 172), DTK-4 (SEQ ID NO: 173), and DTK-5 (SEQ ID NO: 174)), a leucokinin (e.g., LK-I (SEQ ID NO: 175), LK-V (SEQ ID NO: 176), LK-VI (SEQ ID NO: 177), and LK-VIII (SEQ ID NO: 178), Culekinin (SEQ ID NO: 179), mollusc leucokinin-like peptide, lymnokinin (PSFHSWSa) (SEQ ID NO: 180), DLK-1 (SEQ ID NO: 181), DLK-2 (SEQ ID NO: 182), and DLK-2A (QRFHSWGa) (SEQ ID NO: 183)), or an allatostatin (AST-C (SEQ ID NO: 184 or DST-C SEQ ID NO: 185)). Other binding partners include, without limitation, SEQ ID NO: 186 and SEQ ID NO: 187. The DmGPCR modulator may be an anti-DmGPCR antibody or a DmGPCR antisense polynucleotide.

Another embodiment of the invention provides methods of preventing or treating a disease or condition caused by an ectoparasite in a host subject by administering to the subject a binding partner or modulator of a DmGPCR polynucleotide or polypeptide to modify the expression or avtivity of the DmGPCR.

Additional features and variations of the invention will be apparent to those skilled in the art from the entirety of this application, including the detailed description, and all such features are intended as aspects of the invention. Likewise, features of the invention described herein can be re-combined into additional embodiments that also are intended as aspects of the invention, irrespective of whether the combination of features is specifically mentioned above as an aspect or embodiment of the invention. Also, only such limitations which are described herein as critical to the invention should be viewed as such; variations of the invention lacking limitations which have not been described herein as critical are intended as aspects of the invention.

### DETAILED DESCRIPTION OF PREFERRED EMBODIMENTS

The present invention provides, inter alia, isolated and purified polynucleotides that encode D. melanogaster G protein coupled receptor (DmGPCR) or a portion thereof, vectors containing these polynucleotides, host cells transformed with these vectors, processes of making DmGPCR, methods of using the above polynucleotides and vectors, isolated and purified DmGPCR, methods of screening compounds which modulate DmGPCR activity, and methods of identifying mammalian, vertebrate, or invertebrate homologs of DmGPCR.

Various definitions are made throughout this document. Most words have the meaning that would be attributed to those words by one skilled in the art. Words specifically defined either below or elsewhere in this document have the meaning provided in the context of the present invention as a whole and as are typically understood by those skilled in the art.

It is to be understood that when groups of sequences are set forth, combinations and sub-combinations thereof are also specifically contemplated. For example, with the disclosure of “SEQ ID NOs: 1, 3, 5, 7, 9, 11, 13, 15, 17, 19, 21, or 23”, it is to be understood that the present invention includes combinations and subcombinations, including but not limited to, SEQ ID NOs: 1 and 3; 1 and 5; 1, 3, and 5; etc.

“Synthesized” as used herein and understood in the art, refers to polynucleotides produced by purely chemical, as opposed to enzymatic, methods. “Wholly” synthesized DNA sequences are therefore produced entirely by chemical means, and “partially” synthesized DNAs embrace those wherein only portions of the resulting DNA were produced by chemical means.

By the term “region” is meant a physically contiguous portion of the primary structure of a biomolecule. In the case of proteins, a region is defined by a contiguous portion of the amino acid sequence of that protein.

The term “domain” is herein defined as referring to a structural part of a biomolecule that contributes to a known or suspected function of the biomolecule. Domains may be co-extensive with regions or portions thereof; domains may also incorporate a portion of a biomolecule that is distinct from a particular region, in addition to all or part of that region. Examples of GPCR protein domains include, but are not limited to, the extracellular (i.e., N-terminal), transmembrane and cytoplasmic (i.e., C-terminal) domains, which are co-extensive with like-named regions of GPCRs; each of the seven transmembrane segments of a GPCR; and each of the loop segments (both extracellular and intracellular loops) connecting adjacent transmembrane segments.

As used herein, the term “activity” refers to a variety of measurable indicia suggesting or revealing binding, either direct or indirect; affecting a response, i.e., having a measurable effect in response to some exposure or stimulus, including, for example, the affinity of a compound for directly binding a polypeptide or polynucleotide of the invention, or, for example, measurement of amounts of upstream or downstream proteins or other similar functions after some stimulus or event.

As used herein, the term “antibody” is meant to refer to complete, intact antibodies, and Fab, Fab′, F(ab′)₂, Fᵥ, and other fragments thereof. Complete, intact antibodies include monoclonal antibodies such as murine monoclonal antibodies, chimeric antibodies, human antibodies, and humanized antibodies.

As used herein, the term “binding” means the physical or chemical interaction between two proteins or compounds or associated proteins or compounds or combinations thereof. Binding includes ionic, non-ionic, hydrogen bonds, van der Waals, hydrophobic interactions, etc. The physical interaction, the binding, can be either direct or indirect, indirect being through or due to the effects of another protein or compound. Direct binding refers to interactions that do not take place through or due to the effect of another protein or compound but instead are without other substantial chemical intermediates.

As used herein, the term “compound” means any identifiable chemical or molecule, including, but not limited to, small molecule, peptide, protein, sugar, nucleotide, or nucleic acid, and such compound can be natural or synthetic.

As used herein, the term “complementary” refers to Watson-Crick basepairing between nucleotide units of a nucleic acid molecule.

As used herein, the term “contacting” means bringing together, either directly or indirectly, a compound into physical proximity to a polypeptide or polynucleotide of the invention. The polypeptide or polynucleotide can be in any number of buffers, salts, solutions etc. Contacting includes, for example, placing the compound into a beaker, microtiter plate, cell culture flask, or a microarray, such as a gene chip, or the like, which contains the nucleic acid molecule, or polypeptide encoding the GPCR or fragment thereof.

As used herein, the phrase “homologous nucleotide sequence,” or “homologous amino acid sequence,” or variations thereof, refers to sequences characterised by a homology, at the nucleotide level or amino acid level, of at least the specified percentage. Homologous nucleotide sequences include those sequences coding for isoforms of proteins. Such isoforms can be expressed in different tissues of the same organism as a result of, for example, alternative splicing of RNA. Alternatively, isoforms can be encoded by different genes. Homologous nucleotide sequences include nucleotide sequences encoding for a protein of a species other than insects, including, but not limited to, mammals. Homologous nucleotide sequences also include, but are not limited to, naturally occurring allelic variations and mutations of the nucleotide sequences set forth herein. A homologous nucleotide sequence does not, however, include the nucleotide sequence encoding other known GPCRs. Homologous amino acid sequences include those amino acid sequences which encode conservative amino acid substitutions, as well as polypeptides having neuropeptide binding and/or signalling activity. A homologous amino acid sequence does not, however, include the amino acid sequence encoding other known GPCRs. Percent homology can be determined by, for example, the Gap program (Wisconsin Sequence Analysis Package, Version 8 for Unix, Genetics Computer Group, University Research Park, Madison, Wis.), using the default settings, which uses the algorithm of Smith and Waterman (Adv. Appl. Math., 1981, 2, 482-489, which is incorporated herein by reference in its entirety).

As used herein, the term “isolated” nucleic acid molecule refers to a nucleic acid molecule (DNA or RNA) that has been removed from its native environment. Examples of isolated nucleic acid molecules include, but are not limited to, recombinant DNA molecules contained in a vector, recombinant DNA molecules maintained in a heterologous host cell, partially or substantially purified nucleic acid molecules, and synthetic DNA or RNA molecules.

As used herein, the terms “regulates”, “modulates”, or “modifies” means an increase or decrease in the amount, quality, or effect of a particular activity or protein.

As used herein, the term “enhanced activity” means increased activity. The term “impaired activity” means decreased activity.

As used herein, the term “oligonucleotide” refers to a series of linked nucleotide residues which has a sufficient number of bases to be used in a polymerase chain reaction (PCR). This short sequence is based on (or designed from) a genomic or cDNA sequence and is used to amplify, confirm, or reveal the presence of an identical, similar or complementary DNA or RNA in a particular cell or tissue. Oligonucleotides comprise portions of a DNA sequence having at least about 10 nucleotides and as many as about 50 nucleotides, preferably about 15 to 30 nucleotides. They are chemically synthesized and may be used as probes.

As used herein, the term “probe” refers to nucleic acid sequences of variable length, preferably between at least about 10 and as many as about 6,000 nucleotides, depending on use. They are used in the detection of identical, similar, or complementary nucleic acid sequences. Longer length probes are usually obtained from a natural or recombinant source, are highly specific and much slower to hybridize than oligomers. They may be single- or double-stranded and carefully designed to have specificity in PCR, hybridization membrane-based, or ELISA-like technologies.

“Portion” or “fragment” when referring to a polynucleotide includes a polynucleotide sequence having at least 14, 16, 18, 20, 25, 50, or 75 consecutive nucleotides of the reference polynucleotide from which the fragment or portion is derived. “Portion” or “fragment” when referring to a polypeptide refers to a polypeptide having at least 5, 10, 15, 20, 25, 30, 35, or 40 consecutive amino acids of the reference polypeptide from which the fragment is derived.

The term “preventing” refers to decreasing the probability that an organism contracts or develops an abnormal condition.

The phrase “controlling an insect population” or variants thereof refers to an increase or decrease in the number of insects in the population. For example, methods of controlling an insect population include methods of increasing the number of beneficial insects in a given insect population and methods of decreasing the number of harmful insects in a given insect population.

The term “treating” refers to having a therapeutic effect and at least partially alleviating or abrogating an abnormal condition in the organism.

The term “subject” as used herein refers to insects, vertebrates, invertebrates, and mammals.

The term “therapeutic effect” refers to the inhibition or activation of factors causing or contributing to an abnormal or normal condition. A therapeutic effect relieves to some extent one or more of the symptoms of the abnormal or normal condition. A therapeutic effect can refer to one or more of the following: (a) an increase in the proliferation, growth, and/or differentiation of cells; (b) inhibition (i.e., slowing or stopping) of cell death; (c) inhibition of degeneration; (d) relieving to some extent one or more of the symptoms associated with the condition; and (e) enhancing the function of the affected population of cells. Compounds demonstrating efficacy against abnormal or normal conditions can be identified as described herein.

A condition of an organism to be treated may be abnormal or normal. The term “abnormal condition” refers to a function in the cells or tissues of an organism that deviates from their normal functions in that organism. For example, abnormal condition can relate to cell proliferation, cell differentiation, cell signaling, or cell survival.

The phrase “normal condition” refers to a normal function in the cells or tissue of an organism. For example, a normal condition can relate to cell proliferation, cell differentiation, cell signaling, or cell survival.

The term “administering” relates to a method of incorporating a compound into cells or tissues of an organism. A condition can be prevented, treated, or induced when the cells or tissues of the organism exist within the organism or outside of the organism. Cells existing outside the organism can be maintained or grown in cell culture dishes. For cells harbored within the organism, many techniques exist in the art to administer compounds, including (but not limited to) oral, parenteral, dermal, injection, and aerosol applications. For cells outside of the organism, multiple techniques exist in the art to administer the compounds, including (but not limited to) cell microinjection techniques, transformation techniques and carrier techniques.

The condition can also be prevented, treated, or induced by administering a compound to a group of cells having to modify a signal transduction pathway of a subject organism. The effect of administering a compound on organism function can then be monitored. The subject may be, for example, a mammal, such as a mouse, rat, rabbit, guinea pig, companion animal (such as a dog or cat), livestock animal (such as a chicken, pig, or cow), goat, horse, monkey, ape, or human; a worm; or an insect.

By “amplification” it is meant increased numbers of DNA or RNA in a cell compared with normal cells. “Amplification” as it refers to RNA can be the detectable presence of RNA in cells, since in some normal cells there is no basal expression of RNA. In other normal cells, a basal level of expression exists, therefore in these cases amplification is the detection of at least 1-2-fold, and preferably more, compared to the basal level.

As used herein, the phrase “stringent hybridization conditions” or “stringent conditions” refers to conditions under which a probe, primer, or oligonucleotide will hybridize to its target sequence, but to no other sequences. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present in excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 to 50 nucleotides) and at least about 60° C. for longer probes, primers or oligonucleotides. Stringent conditions may also be achieved with the addition of destabilizing agents, such as formamide.

The amino acid sequences are presented in the amino to carboxy direction, from left to right. The amino and carboxy groups are not presented in the sequence. The nucleotide sequences are presented by single strand only, in the 5′ to 3′ direction, from left to right. Nucleotides and amino acids are represented in the manner recommended by the IUPAC-IUB Biochemical Nomenclature Commission, or (for amino acids) by three letters code.

Polynucleotides

Genomic DNA of the invention comprises the protein-coding region for a polypeptide of the invention and is also intended to include allelic variants thereof. It is widely understood that, for many genes, genomic DNA is transcribed into RNA transcripts that undergo one or more splicing events wherein introns (i.e., non-coding regions) of the transcripts are removed, or “spliced out.” RNA transcripts that can be spliced by alternative mechanisms, and therefore are subject to removal of different RNA sequences but still encode a DmGPCR polypeptide, are referred to in the art as “splice variants” which are embraced by the invention. Splice variants comprehended by the invention therefore are encoded by the same original genomic DNA sequences but arise from distinct mRNA transcripts. Allelic variants are modified forms of a wild-type gene sequence, the modification resulting from recombination during chromosomal segregation or exposure to conditions which give rise to genetic mutation. Allelic variants, like wild type genes, are naturally occurring sequences (as opposed to non-naturally occurring variants which arise from in vitro manipulation).

The invention also comprehends cDNA that is obtained through reverse transcription of an RNA polynucleotide encoding DmGPCR (conventionally followed by second strand synthesis of a complementary strand to provide a double-stranded DNA).

A DNA sequence encoding a DmGPCR polypeptide is set out in any of SEQ ID NOs: 1, 3, 5, 7, 9, 11, 13, 15, 17, 19, 21, or 23. A DNA of the invention may comprise a double stranded molecule along with the complementary molecule (the “non-coding strand” or “complement”) having a sequence unambiguously deducible from the coding strand according to Watson-Crick base-pairing rules for DNA. Also included in the invention are other polynucleotides encoding any of the particular DmGPCR polypeptides of the invention which differ in sequence from the particular polynucleotides described herein by virtue of the well-known degeneracy of the universal nuclear genetic code.

The invention further embraces species, such as mammalian, homologs of the DmGPCR DNA. Species homologs, sometimes referred to as “orthologs,” in general, share at least 35%, at least 40%, at least 45%, at least 50%, at least 60%, at least 65%, at least 70%, at least 75%, at least 80%, at least 85%, at least 90%, at least 95%, at least 98%, or at least 99% homology with DNA of the invention. Generally, percent sequence “homology” with respect to polynucleotides of the invention may be calculated as the percentage of nucleotide bases in the candidate sequence that are identical to nucleotides in the DmGPCR sequence set forth in a particular polynucleotide sequence, after aligning the sequences and introducing gaps, if necessary, to achieve the maximum percent sequence identity.

Another aspect of the present invention is the use of the DmGPCR nucleotide sequences disclosed herein for identifying homologs of the DmGPCR, in other animals, including mammals, vertebrates, and invertebrates. Any of the nucleotide sequences disclosed herein, or any portion thereof, can be used, for example, as probes to screen databases or nucleic acid libraries, such as, for example, genomic or cDNA libraries, to identify homologs, using screening procedures well-known to those skilled in the art.

The polynucleotide sequence information provided by the invention makes possible large-scale expression of the encoded polypeptide by techniques well-known and routinely practiced in the art. Polynucleotides of the invention also permit identification and isolation of polynucleotides encoding related DmGPCR polypeptides, such as allelic variants and species homologs, by well-known techniques including Southern and/or Northern hybridization, and polymerase chain reaction (PCR). Examples of related polynucleotides include genomic sequences, including allelic variants, as well as polynucleotides encoding polypeptides homologous to DmGPCR and structurally related polypeptides sharing one or more biological, immunological, and/or physical properties of DmGPCR. Genes encoding proteins homologous to DmGPCR can also be identified by Southern and/or PCR analysis and are useful in animal models for GPCR disorders. Knowledge of the sequence of a DmGPCR DNA also makes possible through use of Southern hybridization or polymerase chain reaction (PCR) the identification of genomic DNA sequences encoding DmGPCR expression control regulatory sequences such as promoters, operators, enhancers, repressors, and the like. Polynucleotides of the invention are also useful in hybridization assays to detect the capacity of cells to express DmGPCR. Polynucleotides of the invention may also provide a basis for diagnostic methods useful for identifying the presence of an ectoparasite expressing a DmGPCR that underlies a disease state or states, which information is useful both for diagnosis and for selection of therapeutic strategies.

The disclosure herein of a full-length polynucleotide encoding a DmGPCR polypeptide makes readily available to the worker of ordinary skill in the art every possible fragment of the full length polynucleotide. The invention therefore provides fragments of DmGPCR-encoding polynucleotides comprising at least 14, and preferably at least 16, 18, 20, 25, 50, or 75 consecutive nucleotides of a polynucleotide encoding DmGPCR. Fragment polynucleotides of the invention may comprise sequences unique to the DmGPCR-encoding polynucleotide sequence, and therefore hybridize under highly stringent or moderately stringent conditions only (i.e., “specifically”) to polynucleotides encoding DmGPCR (or fragments thereof). Polynucleotide fragments of genomic sequences of the invention comprise not only sequences unique to the coding region, but also include fragments of the full-length sequence derived from introns, regulatory regions, and/or other non-translated sequences. Sequences unique to polynucleotides of the invention are recognizable through sequence comparison to other known polynucleotides, and can be identified through use of alignment programs routinely utilized in the art, e.g., those made available in public sequence databases. Such sequences also are recognizable from Southern hybridization analyses to determine the number of fragments of genomic DNA to which a polynucleotide will hybridize. Polynucleotides of the invention can be labeled in a manner that permits their detection, including radioactive, fluorescent, and enzymatic labeling.

Fragment polynucleotides are particularly useful as probes for detection of full-length or fragment DmGPCR polynucleotides. One or more polynucleotides can be included in kits that are used to detect the presence of a polynucleotide encoding DmGPCR, or used to detect variations in a polynucleotide sequence encoding DmGPCR.

The invention also embraces DNAs encoding DmGPCR polypeptides that hybridize under moderately stringent or high stringency conditions to the non-coding strand, or complement, of the polynucleotides in SEQ ID NOs: 1, 3, 5, 7, 9, 11, 13, 15, 17, 19, 21, or 23.

Exemplary highly stringent hybridization conditions are as follows: hybridization at 42° C. in a hybridization solution comprising 50% formamide, 1% SDS, 1 M NaCl, 10% Dextran sulfate, and washing twice for 30 minutes at 60° C. in a wash solution comprising 0.1×SSC and 1% SDS. It is understood in the art that conditions of equivalent stringency can be achieved through variation of temperature and buffer, or salt concentration as described Ausubel et al. (Eds.), PROTOCOLS IN MOLECULAR BIOLOGY, John Wiley & Sons, 1994, pp. 6.0.3-6.4.10. Modifications in hybridization conditions can be empirically determined or precisely calculated based on the length and the percentage of guanosine/cytosine (GC) base pairing of the probe. The hybridization conditions can be calculated as described in Sambrook et al. (Eds.), MOLECULAR CLONING: A LABORATORY MANUAL, Cold Spring Harbor Laboratory Press: Cold Spring Harbor, N.Y., 1989, pp. 9.47-9.51.

With the knowledge of the nucleotide sequence information disclosed in the present invention, one skilled in the art can identify and obtain nucleotide sequences which encode DmGPCRs from different sources (i.e., different tissues or different organisms) through a variety of means well-known to the skilled artisan and as disclosed by, for example, Sambrook et al., MOLECULAR CLONING: A LABORATORY MANUAL, Second Edition, Cold Spring Harbor Press, Cold Spring Harbor, N.Y., 1989, which is incorporated herein by reference in its entirety.

For example, DNA that encodes DmGPCR may be obtained by screening of mRNA, cDNA, or genomic DNA with oligonucleotide probes generated from the DmGPCR gene sequence information provided herein. Probes may be labeled with a detectable group, such as a fluorescent group, a radioactive atom or a chemiluminescent group in accordance with procedures known to the skilled artisan and used in conventional hybridization assays, as described by, for example, Sambrook et al.

A nucleic acid molecule comprising any of the DmGPCR nucleotide sequences described above can alternatively be synthesized by use of the polymerase chain reaction (PCR) procedure, with the PCR oligonucleotide primers produced from the nucleotide sequences provided herein. See U.S. Pat. Nos. 4,683,195 to Mullis et al. and 4,683,202 to Mullis. The PCR reaction provides a method for selectively increasing the concentration of a particular nucleic acid sequence even when that sequence has not been previously purified and is present only in a single copy in a particular sample. The method can be used to amplify either single- or double-stranded DNA. The essence of the method involves the use of two oligonucleotide probes to serve as primers for the template-dependent, polymerase-mediated replication of a desired nucleic acid molecule.

A wide variety of alternative cloning and in vitro amplification methodologies are well-known to those skilled in the art. Examples of these techniques are found in, for example, Berger et al., GUIDE TO MOLECULAR CLONING TECHNIQUES, METHODS IN ENZYMOLOGY 152 Academic Press, Inc., San Diego, Calif. (Berger), which is incorporated herein by reference in its entirety.

The nucleic acid molecules of the present invention, and fragments derived therefrom, are useful for screening for restriction fragment length polymorphisms (RFLPs) and for genetic mapping.

Automated sequencing methods can be used to obtain or verify the nucleotide sequence of DmGPCR. The DmGPCR nucleotide sequences of the present invention are believed to be 100% accurate. However, as is known in the art, nucleotide sequences obtained by automated methods may contain some errors. Nucleotide sequences determined by automation are typically at least about 90%, more typically at least about 95% to at least about 99.9% identical to the actual nucleotide sequence of a given nucleic acid molecule. The actual sequence may be more precisely determined using manual sequencing methods, which are well-known in the art. An error in a sequence which results in an insertion or deletion of one or more nucleotides may result in a frame shift in translation such that the predicted amino acid sequence will differ from that which would be predicted from the actual nucleotide sequence of the nucleic acid molecule, starting at the point of the mutation.

Expression Constructs and Vectors

Autonomously replicating recombinant expression constructs such as plasmid and viral DNA vectors incorporating polynucleotides of the invention are also provided. Vectors are used herein either to amplify DNA or RNA encoding a DmGPCR and/or to express DNA which encodes a DmGPCR. Vectors of the invention include, but are not limited to, plasmids, phages, cosmids, episomes, viral particles, viruses, and integratable DNA fragments (i.e., fragments integratable into the host genome by homologous recombination). Viral particles may include, but are not limited to, adenoviruses, baculoviruses, parvoviruses, herpesviruses, poxyiruses, adeno-associated viruses, Smeliki Forest viruses, vaccinia viruses, and retroviruses. Examples of expression vectors include, but are not limited to, pcDNA3 (Invitrogen) and pSVL (Pharmacia Biotech). Other expression vectors include, but are not limited to, pSPORT vectors, pGEM vectors (Promega), pPROEXvectors (LTI, Bethesda, Md.), Bluescript vectors (Stratagene), pQE vectors (Qiagen), pSE420 (Invitrogen), and pYES2 (Invitrogen).

Expression constructs wherein DmGPCR-encoding polynucleotides are operatively linked to an endogenous or exogenous expression control DNA sequence and a transcription terminator are also provided. Expression control DNA sequences include promoters, enhancers, operators, and regulatory element binding sites generally, and are typically selected based on the expression systems in which the expression construct is to be utilized. Promoter and enhancer sequences are generally selected for the ability to increase gene expression, while operator sequences are generally selected for the ability to regulate gene expression. Expression constructs of the invention may also include sequences encoding one or more selectable markers that permit identification of host cells bearing the construct. Expression constructs may also include sequences that facilitate, and/or promote, homologous recombination in a host cell. Constructs of the invention may also include sequences necessary for replication in a host cell.

Expression constructs may be utilized for production of an encoded protein, but may also be utilized simply to amplify a DmGPCR-encoding polynucleotide sequence. In some embodiments, the vector is an expression vector wherein the polynucleotide of the invention is operably linked to a polynucleotide comprising an expression control sequence. Some expression vectors are replicable DNA constructs in which a DNA sequence encoding a DmGPCR is operably linked or connected to suitable control sequences capable of effecting the expression of the DmGPCR in a suitable host. DNA regions are operably linked or connected when they are functionally related to each other. For example, a promoter is operably linked or connected to a coding sequence if it controls the transcription of the sequence. Amplification vectors do not require expression control domains but rather need only the ability to replicate in a host, usually conferred by an origin of replication, and a selection gene to facilitate recognition of transformants. The need for control sequences in the expression vector will vary depending upon the host selected and the transformation method chosen. Generally, control sequences include a transcriptional promoter, an optional operator sequence to control transcription, a sequence encoding suitable mRNA ribosomal binding and sequences which control the termination of transciption and translation.

Vectors may contain a promoter that is recognized by the host organism. The promoter sequences of the present invention may be prokaryotic, eukaryotic, or viral. Examples of suitable prokaryotic sequences include the PR and PL promoters of bacteriophage lambda (THE BACTERIOPHAGE LAMBDA, Hershey, A. D., Ed., Cold Spring Harbor Press, Cold Spring Harbor, N.Y., 1973, which is incorporated herein by reference in its entirety; LAMBDA II, Hendrix, R. W., Ed., Cold Spring Harbor Press, Cold Spring Harbor, N.Y., 1980, which is incorporated herein by reference in its entirety); the trp, recA, heat shock, and lacZ promoters of E. coli and the SV40 early promoter (Benoist et al., Nature, 1981, 290, 304-310, which is incorporated herein by reference in its entirety). Additional promoters include, but are not limited to, mouse mammary tumor virus, long terminal repeat of human immunodeficiency virus (HIV), maloney virus, cytomegalovirus immediate early promoter, Epstein Barr virus, Rous sarcoma virus, human actin, human myosin, human hemoglobin, human muscle creatine, and human metallothionein.

Additional regulatory sequences may also be included in the vectors of the invention. Examples of suitable regulatory sequences include, for example, the Shine-Dalgamo sequence of the replicase gene of the phage MS-2 and of the gene clI of bacteriophage lambda. The Shine-Dalgamo sequence may be directly followed by a DNA encoding a DmGPCR, resulting in the expression of the mature DmGPCR protein.

Moreover, suitable expression vectors may include an appropriate marker that allows the screening of the transformed host cells. The transformation of the selected host is carried out using any one of the various techniques well-known to the skilled artisan and described in, for example, Sambrook et al., supra.

An origin of replication can also be provided either by construction of the vector to include an exogenous origin or by the host cell chromosomal replication mechanism. If the vector is integrated into the host cell chromosome, the latter may be sufficient. Alternatively, rather than using vectors which contain viral origins of replication, one skilled in the art can transform mammalian cells by the method of co-transformation with a selectable marker and DmGPCR DNA. An example of a suitable marker is dihydrofolate reductase (DHFR) or thymidine kinase (e.g., U.S. Pat. No. 4,399,216).

Nucleotide sequences encoding a DmGPCR may be recombined with vector DNA in accordance with conventional techniques, including blunt-ended or staggered-ended termini for ligation, restriction enzyme digestion to provide appropriate termini, filling in of cohesive ends as appropriate, alkaline phosphatase treatment to avoid undesirable joining, and ligation with appropriate ligases. Techniques for such manipulation are disclosed by Sambrook et al., supra, and are well-known in the art. Methods for construction of mammalian expression vectors are disclosed in, for example, Okayama et al., Mol. Cell. Biol., 1983, 3, 280; Cosman et al., Mol. Immunol. 1986, 23, 935; Cosman et al., Nature, 1984, 312, 768; EP-A-0367566 and WO 91/18982, each of which is incorporated herein by reference in its entirety.

Host Cells

According to another aspect of the invention, host cells are provided, including prokaryotic and eukaryotic cells, comprising a polynucleotide of the invention (or vector of the invention) in a manner which permits expression of the encoded DmGPCR polypeptide. Polynucleotides of the invention may be introduced into the host cell as part of a circular plasmid, or as linear DNA comprising an isolated protein-coding region or a viral vector. Methods for introducing DNA into the host cell that are well-known and routinely practiced in the art include transformation, transfection, electroporation, nuclear injection, or fusion with carriers such as liposomes, micelles, ghost cells, and protoplasts. Expression systems of the invention include bacterial, yeast, fungal, plant, insect, invertebrate, vertebrate, and mammalian cells systems.

The invention provides host cells that are transformed or transfected (stably or transiently) with polynucleotides of the invention or vectors of the invention. As stated above, such host cells are useful for amplifying the polynucleotides and also for expressing the DmGPCR polypeptide or fragment thereof encoded by the polynucleotide.

According to some aspects of the present invention, transformed host cells having an expression vector comprising any of the nucleic acid molecules described above are provided. Expression of the nucleotide sequence occurs when the expression vector is introduced into an appropriate host cell. Suitable host cells for expression of the polypeptides of the invention include, but are not limited to, prokaryotes, yeast, and eukaryotes. If a prokaryotic expression vector is employed, then the appropriate host cell would be any prokaryotic cell capable of expressing the cloned sequences. Suitable prokaryotic cells include, but are not limited to, bacteria of the genera Escherichia, Bacillus, Salmonella, Pseudomonas, Streptomyces, and Staphylococcus.

If an eukaryotic expression vector is employed, then the appropriate host cell would be any eukaryotic cell capable of expressing the cloned sequence. Eukaryotic cells may be cells of higher eukaryotes. Suitable eukaryotic cells include, but are not limited to, non-human mammalian tissue culture cells and human tissue culture cells. Host cells may include, but are not limited to, insect cells, HeLa cells, Chinese hamster ovary cells (CHO cells), African green monkey kidney cells (COS cells), human 293 cells, and murine 3T3 fibroblasts. Propagation of such cells in cell culture has become a routine procedure (see, e.g., TISSUE CULTURE, Academic Press, Kruse and Patterson, eds., 1973, which is incorporated herein by reference in its entirety).

In addition, a yeast host may be employed as a host cell. Examples of yeast cells include, but are not limited to, the genera Saccharomyces, Pichia, and Kluveromyces. Examples of yeast hosts are S. cerevisiae and P. pastoris. Yeast vectors can contain an origin of replication sequence from a 2T yeast plasmid, an autonomously replicating sequence (ARS), a promoter region, sequences for polyadenylation, sequences for transcription termination, and a selectable marker gene. Shuttle vectors for replication in both yeast and E. coli are also included herein.

Alternatively, insect cells may be used as host cells. In one embodiment, the polypeptides of the invention are expressed using a baculovirus expression system (see, Luckow et al., Bio/Technology, 1988, 6, 47, BACULOVIRUS EXPRESSION VECTORS: A LABORATORY MANUAL, O'Rielly et al. (Eds.), W. H. Freeman and Company, New York, 1992, and U.S. Pat. No. 4,879,236, each of which is incorporated herein by reference in its entirety). In addition, the MAXBAC™ complete baculovirus expression system (Invitrogen) can, for example, be used for production in insect cells.

In still another related embodiment, the invention provides methods for producing a DmGPCR polypeptide (or fragment thereof) comprising the steps of growing a host cell of the invention in a nutrient medium and isolating the polypeptide or variant thereof from the cell or the medium. Because DmGPCR is a seven transmembrane receptor, it will be appreciated that, for some applications, such as certain activity assays, isolation may involve isolation of cell membranes containing the polypeptide embedded therein, whereas for other applications a more complete isolation may be desired.

Host cells of the invention are a valuable source of immunogen for development of antibodies specifically immunoreactive with DmGPCR. Host cells of the invention are also useful in methods for the large-scale production of DmGPCR polypeptides wherein the cells are grown in a suitable culture medium and the desired polypeptide products are isolated from the cells, or from the medium in which the cells are grown, by purification methods known in the art, e.g., conventional chromatographic methods including immunoaffinity chromatography, receptor affinity chromatography, hydrophobic interaction chromatography, lectin affinity chromatography, size exclusion filtration, cation or anion exchange chromatography, high pressure liquid chromatography (HPLC), reverse phase HPLC, and the like. Still other methods of purification include those methods wherein the desired protein is expressed and purified as a fusion protein having a specific tag, label, or chelating moiety that is recognized by a specific binding partner or agent. The purified protein can be cleaved to yield the desired protein, or can be left as an intact fusion protein. Cleavage of the fusion component may produce a form of the desired protein having additional amino acid residues as a result of the cleavage process.

Knowledge of DmGPCR DNA sequences allows for modification of cells to permit, or increase, expression of endogenous DmGPCR. Cells can be modified (e.g., by homologous recombination) to provide increased expression by replacing, in whole or in part, the naturally occurring DmGPCR promoter with all or part of a heterologous promoter so that the cells express DmGPCR at higher levels. The heterologous promoter is inserted in such a manner that it is operatively linked to endogenous DmGPCR encoding sequences. (See, e.g., PCT International Publication No. WO 94/12650, PCT International Publication No. WO 92/20808, and PCT International Publication No. WO 91/09955.) It is also contemplated that, in addition to heterologous promoter DNA, amplifiable marker DNA (e.g., ada, dhfr, and the multifunctional CAD gene which encodes carbamoyl phosphate synthase, aspartate transcarbamylase, and dihydroorotase), and/or intron DNA may be inserted along with the heterologous promoter DNA. If linked to the DmGPCR coding sequence, amplification of the marker DNA by standard selection methods results in co-amplification of the DmGPCR coding sequences in the cells.

Knock-Outs

The DNA sequence information provided by the present invention also makes possible the development (e.g., by homologous recombination or “knock-out” strategies; see Capecchi, Science, 1989, 244, 1288-1292) of subjects that fail to express functional DmGPCR or that express a variant of DmGPCR. Such subjects (especially including insects and worms) are useful as models for studying the in vivo activities of DmGPCR and modulators of DmGPCR and are also useful for further elucidating the role of DmGPCRs in insects or worms.

Antisense

Also made available by the invention are antisense polynucleotides which recognize and hybridize to polynucleotides encoding DmGPCR. Full-length and fragment antisense polynucleotides are provided. Fragment antisense molecules of the invention include those which specifically recognize and hybridize to DmGPCR expression control sequences or DmGPCR RNA (as determined by sequence comparison of DNA encoding DmGPCR to DNA encoding other known molecules). Identification of sequences unique to DmGPCR-encoding polynucleotides, can be deduced through use of any publicly available sequence database, and/or through use of commercially available sequence comparison programs. After identification of the desired sequences, isolation through restriction digestion or amplification using any of the various polymerase chain reaction techniques well-known in the art can be performed. Antisense polynucleotides are particularly relevant to regulating expression of DmGPCR by those cells expressing DmGPCR mRNA.

Antisense nucleic acids (preferably 10 to 20 base-pair oligonucleotides) capable of specifically binding to DmGPCR expression control sequences or DmGPCR RNA are introduced into cells (e.g., by a viral vector or colloidal dispersion system such as a liposome). The antisense nucleic acid binds to the DmGPCR target nucleotide sequence in the cell and prevents transcription and/or translation of the target sequence. Phosphorothioate and methylphosphonate antisense oligonucleotides are specifically contemplated for therapeutic use by the invention. The antisense oligonucleotides may be further modified by poly-L-lysine, transferrin polylysine, or cholesterol moieties at their 5′ end. Suppression of DMGPCR expression at either the transcriptional or translational level is useful to generate cellular or animal models for studying the biological role of DmGPCRs.

Antisense oligonucleotides, or fragments of a nucleotide sequence selected from the group consisting of SEQ ID NOs: 1, 3, 5, 7, 9, 11, 13, 15, 17, 19, 21, or 23, or sequences complementary or homologous thereto, derived from the nucleotide sequences of the present invention encoding DmGPCR are useful for probing gene expression in various tissues. For example, tissue can be probed in situ with oligonucleotide probes carrying detectable groups by conventional autoradiography techniques. Antisense oligonucleotides directed to regulatory regions of a nucleotide sequence may be selected from the group consisting of SEQ ID NOs: 1, 3, 5, 7, 9, 11, 13, 15, 17, 19, 21, or 23, or mRNA corresponding thereto, including, but not limited to, the initiation codon, TATA box, enhancer sequences, and the like.

Transcription Factors

The DmGPCR sequences taught in the present invention facilitate the design of novel transcription factors for modulating DmGPCR expression in native cells and subjects, and cells transformed or transfected with DmGPCR polynucleotides. For example, the Cys₂-His₂ zinc finger proteins, which bind DNA via their zinc finger domains, have been shown to be amenable to structural changes that lead to the recognition of different target sequences. These artificial zinc finger proteins recognize specific target to sites with high affinity and low dissociation constants, and are able to act as gene switches to modulate gene expression. Knowledge of the particular DmGPCR target sequence of the present invention facilitates the engineering of zinc finger proteins specific for the target sequence using known methods such as a combination of structure-based modeling and screening of phage display libraries (Segal et al., Proc. Natl. Acad. Sci. USA, 1999, 96, 2758-2763; Liu et al., Proc. Natl. Acad. Sci. USA, 1997, 94, 5525-5530 (1997); Greisman et al., Science, 1997, 275, 657-661; Choo et al., J. Mol. Biol., 1997, 273, 525-532). Each zinc finger domain usually recognizes three or more base pairs. Since a recognition sequence of 18 base pairs is generally sufficient in length to render it unique in any known genome, a zinc finger protein consisting of 6 tandem repeats of zinc fingers would be expected to ensure specificity for a particular sequence (Segal et al.). The artificial zinc finger repeats, designed based on DmGPCR sequences, are fused to activation or repression domains to promote or suppress DmGPCR expression (Liu et al.). Alternatively, the zinc finger domains can be fused to the TATA box-binding factor (TBP) with varying lengths of linker region between the zinc finger peptide and the TBP to create either transcriptional activators or repressors (Kim et al., Proc. Natl. Acad. Sci. USA, 1997, 94, 3616-3620). Such proteins, and polynucleotides that encode them, have utility for modulating DmGPCR expression in vivo. The novel transcription factor can be delivered to the target cells by transfecting constructs that express the transcription factor (gene therapy), or by introducing the protein. Engineered zinc finger proteins can also be designed to bind RNA sequences for use in therapeutics as alternatives to anti sense or catalytic RNA methods (McColl et al., Proc. Natl. Acad. Sci. USA, 1997, 96, 9521-9526; Wu et al., Proc. Natl. Acad. Sci. USA, 1995, 92, 344-348). The present invention contemplates methods of designing such transcription factors based on the gene sequence of the invention, as well as customized zinc finger proteins, that are useful to modulate DmGPCR expression in cells (native or transformed) whose genetic complement includes these sequences.

Polypeptides

The invention also provides purified and isolated DmGPCR polypeptides encoded by a polynucleotide of the invention including a DmGPCR polypeptide comprising the amino acid sequence set out in any of SEQ ID NOs: 2, 4, 6, 8, 10, 12, 14, 16, 18, 20, 22, or 24.

It will be appreciated that extracellular epitopes are particularly useful for generating and screening for antibodies and other binding compounds that bind to receptors such as DmGPCR. Thus, in another embodiment, the invention provides purified and isolated polypeptides comprising at least one extracellular domain (e.g., the N-terminal extracellular domain or one of the three extracellular loops) of DmGPCR, such as the N-terminal extracellular domain of DmGPCR. Also included within the scope of the invention are purified and isolated polypeptides comprising a DmGPCR fragment selected from the group consisting of transmembrane domains of DmGPCR, an extracellular loop connecting transmembrane domains of DmGPCR, an intracellular loop connecting transmembrane domains of DmGPCR, the C-terminal cytoplasmic region of DmGPCR, and fusions thereof. Such fragments may be continuous portions of the native receptor. However, it will also be appreciated that knowledge of the DmGPCR gene and protein sequences as provided herein permits recombining of various domains that are not contiguous in the native protein. Using a FORTRAN computer program called “tmtrest.all” (Parodi et al., Comput. Appl. Biosci., 1994, 5, 527-535), DmGPCR was shown to contain transmembrane-spanning domains.

The invention also embraces polypeptides that have at least 99%, at least 95%, at least 90%, at least 85%, at least 80%, at least 75%, at least 70%, at least 65%, at least 60%, at least 55%, or at least 50% identity and/or homology to the reference polypeptide of the invention. Percent amino acid sequence “identity” with respect to the reference polypeptide of the invention is defined herein as the percentage of amino acid residues in the candidate sequence that are identical with the residues in the DmGPCR sequence after aligning both sequences and introducing gaps, if necessary, to achieve the maximum percent sequence identity, and not considering any conservative substitutions as part of the sequence identity. Percent sequence “homology” with respect to the reference polypeptide of the invention is defined herein as the percentage of amino acid residues in the candidate sequence that are identical with the residues in the DmGPCR sequence after aligning the sequences and introducing gaps, if necessary, to achieve the maximum percent sequence identity, and also considering any conservative substitutions as part of the sequence identity.

In one aspect, percent homology is calculated as the percentage of amino acid residues in the smaller of two sequences which align with identical amino acid residue in the sequence being compared, when four gaps in a length of 100 amino acids may be introduced to maximize alignment (Dayhoff, in ATLAS OF PROTEIN SEQUENCE AND STRUCTURE, vol. 5, National Biochemical Research Foundation, Washington, D.C., 1972, p. 124, incorporated herein by reference).

Polypeptides of the invention may be isolated from natural cell sources or may be chemically synthesized, and may be produced by recombinant procedures involving host cells of the invention. Use of mammalian host cells is expected to provide for such post-translational modifications (e.g., glycosylation, truncation, lipidation, and phosphorylation) as may be needed to confer optimal biological activity on recombinant expression products of the invention. Glycosylated and non-glycosylated forms of DmGPCR polypeptides are embraced by the invention.

The invention also embraces variant (or analog) DmGPCR polypeptides. In one example, insertion variants are provided wherein one or more amino acid residues supplement a DmGPCR amino acid sequence. Insertions may be located at either or both termini of the protein, or may be positioned within internal regions of the DmGPCR amino acid sequence. Insertional variants with additional residues at either or both termini can include, for example, fusion proteins and proteins including amino acid tags or labels.

Insertion variants include DmGPCR polypeptides wherein one or more amino acid residues are added to a DmGPCR acid sequence, or to a biologically active fragment thereof.

Variant products of the invention also include mature DmGPCR products, i.e., DmGPCR products wherein leader or signal sequences are removed, with additional amino terminal residues. The additional amino terminal residues may be derived from another protein, or may include one or more residues that are not identifiable as being derived from specific proteins. DmGPCR products with an additional methionine residue at position −1 (Met⁻¹-DmGPCR) are contemplated, as are variants with additional methionine and lysine residues at positions −2 and −1 (Met⁻²-Lys⁻¹-DmGPCR). Variants of DmGPCR with additional Met, Met-Lys, Lys residues (or one or more basic residues in general) are particularly useful for enhanced recombinant protein production in bacterial host cells.

The invention also embraces DmGPCR variants having additional amino acid residues which result from use of specific expression systems. For example, use of commercially available vectors that express a desired polypeptide as part of a glutathione-S-transferase (GST) fusion product provides the desired polypeptide having an additional glycine residue at position −1 after cleavage of the GST component from the desired polypeptide. Variants which result from expression in other vector systems are also contemplated.

Insertional variants also include fusion proteins wherein the amino terminus and/or the carboxy terminus of DmGPCR is/are fused to another polypeptide.

In another aspect, the invention provides deletion variants wherein one or more amino acid residues in a DmGPCR polypeptide are removed. Deletions can be effected at one or both termini of the DmGPCR polypeptide, or with removal of one or more non-terminal amino acid residues of DmGPCR. Deletion variants, therefore, include all fragments of a DmGPCR polypeptide.

The invention also embraces polypeptide fragments of the sequence set out in any of SEQ ID NOs: 2, 4, 6, 8, 10, 12, 14, 16, 18, 20, 22, or 24 wherein the fragments maintain biological (e.g., ligand binding and/or intracellular signaling) and immunological properties of a DmGPCR polypeptide. Fragments comprising at least 5, 10, 15, 20, 25, 30, 35, or 40 consecutive amino acids of any of the polypeptides described herein are comprehended by the invention. Polypeptide fragments may display antigenic properties unique to, or specific for, DmGPCR and its allelic and species homologs. Fragments of the invention having the desired biological and immunological properties can be prepared by any of the methods well-known and routinely practiced in the art.

In still another aspect, the invention provides substitution variants of DmGPCR polypeptides. Substitution variants include those polypeptides wherein one or more amino acid residues of a DmGPCR polypeptide are removed and replaced with alternative residues. In one aspect, the substitutions are conservative in nature; however, the invention embraces substitutions that are also non-conservative. Conservative substitutions for this purpose may be defined as set out in Tables 1, 2, or 3 below.

Variant polypeptides include those wherein conservative substitutions have been introduced by modification of polynucleotides encoding polypeptides of the invention. Amino acids can be classified according to physical properties and contribution to secondary and tertiary protein structure. A conservative substitution is recognized in the art as a substitution of one amino acid for another amino acid that has similar properties. Exemplary conservative substitutions are set out in Table 1 (from WO 97/09433, page 10, published Mar. 13, 1997 (PCT/GB96/02197, filed Sep. 6, 1996)), immediately below.

TABLE 1
Conservative Substitutions I

| SIDE CHAIN | |
|---|---|
| CHARACTERISTIC | AMINO ACID |
| Aliphatic | |
| Non-polar | G A P |
| | I L V |
| Polar - uncharged | C S T M |
| | N Q |
| Polar - charged | D E |
| | K R |
| Aromatic | H F W Y |
| Other | N Q D E |

Alternatively, conservative amino acids can be grouped as described in Lehninger, (BIOCHEMISTRY, Second Edition; Worth Publishers, Inc. NY, N.Y., 1975, pp.71-77) as set out in Table 2, immediately below.

TABLE 2
Conservative Substitutions II

| SIDE CHAIN | |
|---|---|
| CHARACTERISTIC | AMINO ACID |
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

As still another alternative, exemplary conservative substitutions are set out in Table 3, below.

TABLE 3
Conservative Substitutions III

| Original | |
|---|---|
| Residue | Exemplary Substitution |
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr |
| Tyr (Y) | Trp, Phe, Thr, Ser |
| Val (V) | Ile, Leu, Met, Phe, Ala |

It should be understood that the definition of polypeptides of the invention is intended to include polypeptides bearing modifications other than insertion, deletion, or substitution of amino acid residues. By way of example, the modifications may be covalent in nature, and include for example, chemical bonding with polymers, lipids, other organic, and inorganic moieties. Such derivatives may be prepared to increase circulating half-life of a polypeptide, or may be designed to improve the targeting capacity of the polypeptide for desired cells, tissues, or organs. Similarly, the invention further embraces DmGPCR polypeptides that have been covalently modified to include one or more water-soluble polymer attachments such as polyethylene glycol, polyoxyethylene glycol, or polypropylene glycol.

Variants that display ligand binding properties of native DmGPCR and are expressed at higher levels, as well as variants that provide for constitutively active receptors, are particularly useful in assays of the invention; the variants are also useful in assays of the invention and in providing cellular, tissue and animal models for studying aberrant DmGPCR activity.

Antibodies

Also comprehended by the present invention are antibodies (e.g., monoclonal and polyclonal antibodies, single chain antibodies, chimeric antibodies, bifunctional/bispecific antibodies, humanized antibodies, human antibodies, and complementary determining region (CDR)-grafted antibodies, including compounds which include CDR sequences which specifically recognize a polypeptide of the invention) specific for DmGPCR or fragments thereof. Antibody fragments, including Fab, Fab′, F(ab′)₂, and Fᵥ, are also provided by the invention. The term “specific for,” when used to describe antibodies of the invention, indicates that the variable regions of the antibodies of the invention recognize and bind DmGPCR polypeptides exclusively (i.e., are able to distinguish DmGPCR polypeptides from other known GPCR polypeptides by virtue of measurable differences in binding affinity, despite the possible existence of localized sequence identity, homology, or similarity between DmGPCR and such polypeptides). It will be understood that specific antibodies may also interact with other proteins (for example, S. aureus protein A or other antibodies in ELISA techniques) through interactions with sequences outside the variable region of the antibodies, and, in particular, in the constant region of the molecule. Screening assays to determine binding specificity of an antibody of the invention are well-known and routinely practiced in the art. For a comprehensive discussion of such assays, see Harlow et al. (Eds.), ANTIBODIES A LABORATORY MANUAL, Cold Spring Harbor Laboratory, Cold Spring Harbor, N.Y., 1988, Chapter 6. Antibodies that recognize and bind fragments of the DmGPCR polypeptides of the invention are also contemplated, provided that the antibodies are specific for DmGPCR polypeptides. Antibodies of the invention can be produced using any method well-known and routinely practiced in the art.

The invention provides antibodies that are specific for the DmGPCR of the invention. Antibody specificity is described in greater detail below. However, it should be emphasized that antibodies that can be generated from polypeptides that have previously been described in the literature and that are capable of fortuitously cross-reacting with DmGPCR (e.g., due to the fortuitous existence of a similar epitope in both polypeptides) are considered “cross-reactive” antibodies. Such cross-reactive antibodies are not antibodies that are “specific” for DmGPCR. The determination of whether an antibody is specific for DmGPCR or is cross-reactive with another known receptor is made using any of several assays, such as Western blotting assays, that are well-known in the art. For identifying cells that express DmGPCR and also for modulating DmGPCR-ligand binding activity, antibodies that specifically bind to an extracellular epitope of the DmGPCR are useful.

In one variation, the invention provides monoclonal antibodies. Hybridomas that produce such antibodies also are intended as aspects of the invention. In yet another variation, the invention provides a humanized antibody. Humanized antibodies are useful for in vivo therapeutic indications for treatment of diseases or conditions caused by ectoparasites.

In another variation, the invention provides a cell-free composition comprising polyclonal antibodies, wherein at least one of the antibodies is an antibody of the invention specific for DmGPCR. Antisera isolated from an animal is an exemplary composition, as is a composition comprising an antibody fraction of an antisera that has been resuspended in water or in another diluent, excipient, or carrier.

In still another related embodiment, the invention provides anti-idiotypic antibodies specific for an antibody that is specific for DmGPCR.

It is well-known that antibodies contain relatively small antigen binding domains that can be isolated chemically or by recombinant techniques. Such domains are useful DmGPCR binding molecules themselves, and also may be fused to toxins or other polypeptides. Thus, in still another embodiment, the invention provides a polypeptide comprising a fragment of a DmGPCR-specific antibody, wherein the fragment and the polypeptide bind to the DmGPCR. By way of non-limiting example, the invention provides polypeptides that are single chain antibodies, CDR-grafted antibodies, and humanized antibodies.

Non-human antibodies may be humanized by any of the methods known in the art. In one method, the non-human CDRs are inserted into a human antibody or consensus antibody framework sequence. Further changes can then be introduced into the antibody framework to modulate affinity or immunogenicity.

Antibodies of the invention are useful for, e.g., therapeutic purposes (by modulating activity of ectoparasitic DmGPCR), diagnostic purposes to detect or quantitate ectoparasitic DmGPCR, and purification of DmGPCR. Kits comprising an antibody of the invention for any of the purposes described herein are also comprehended. In general, a kit of the invention also includes a control antigen for which the antibody is immunospecific.

The invention also provides methods of using antibodies of the invention. For example, the invention provides methods for modulating ligand binding of a DmGPCR comprising the step of contacting the DmGPCR with an antibody specific for the DmGPCR, under conditions wherein the antibody binds the receptor. The antibodies of the invention may be used to control an insect population by administering an anti-DmGPCR antibody to an insect to modulate ligand binding of the DmGPCR. For example, the insects may be selected from flies, fruitflies, ticks, lice, fleas, cockroaches, and mites.

Gene Manipulation

Gene manipulation using DmGPCR is also useful in subjects such as insects. Gene manipulation includes restoration of DmGPCR activity, DmGPCR overexpression, and negative regulation of DmGPCR. The present invention also comprehends gene manipulation to restore DmGPCR activity lost due to a loss of function mutation. Delivery of a functional DmGPCR gene to appropriate cells is effected ex vivo, in situ, or in vivo by use of vectors, and more particularly viral vectors (e.g., adenovirus, adeno-associated virus, or a retrovirus), or ex vivo by use of physical DNA transfer methods (e.g., liposomes or chemical treatments). See, e.g., Anderson, Nature, 1998, suppl. 392 (6679), 25-20. For additional reviews of gene therapy technology see Friedmann, Science, 1989, 244, 1275-1281; Verma, Scientific American, 1990, 68-84; and Miller, Nature, 1992, 357, 455-460. It is also contemplated that gene manipulation, for example antisense treatment, could be applied to negatively regulate the expression of DmGPCR. As a non-limiting example, gene manipulation may be useful for controlling an insect population by knocking-out or downregulating one or more DmGPCR genes or fragments thereof (see supra and infra).

Compositions

Another aspect of the present invention is directed to compositions, including insecticidal and pharmaceutical compositions, comprising any of the nucleic acid molecules or recombinant expression vectors described above and an acceptable carrier or diluent. The carrier or diluent may be pharmaceutically acceptable. Suitable carriers are described in the most recent edition of REMINGTON'S PHARMACEUTICAL SCIENCES, A. Osol, a standard reference text in this field, which is incorporated herein by reference in its entirety. Examples of such carriers or diluents include, but are not limited to, water, saline, Ringer's solution, dextrose solution, and 5% human serum albumin. Liposomes and nonaqueous vehicles such as fixed oils may also be used. The formulations are sterilized by commonly used techniques.

Also within the scope of the invention are compositions comprising polypeptides, polynucleotides, or antibodies of the invention that have been formulated with, e.g., a pharmaceutically acceptable carrier.

The invention provides insecticidal compositions comprising a DmGPCR polynucleotide, a DmGPCR polypeptide, an anti-DmGPCR antibody, fragments or portions thereof having DmGPCR-binding activity, a DmGPCR binding partner, or a DmGPCR modulator.

Kits and Methods

The present invention is also directed to kits, including pharmaceutical and insecticidal kits. The kits can comprise any of the nucleic acid molecules described above, any of the polypeptides described above, or any antibody which binds to a polypeptide of the invention as described above, as well as a negative control. The kit may comprise additional components, such as, for example, instructions, solid support, reagents helpful for quantification, and the like.

Kits may be designed to detect either expression of polynucleotides or the encoded proteins. For example, oligonucleotide hybridization kits can be provided which include a container having an oligonucleotide probe specific for the DmGPCR-specific DNA and optionally, containers with positive and negative controls and/or instructions. Similarly, PCR kits can be provided which include a container having primers specific for the DmGPCR-specific sequences, DNA and optionally, containers with size markers, positive and negative controls and/or instructions.

Hybridization conditions should be such that hybridization occurs only with the genes in the presence of other nucleic acid molecules. Under stringent hybridization conditions only highly complementary nucleic acid sequences hybridize. Such conditions may prevent hybridization of nucleic acids having 1 or 2 mismatches out of 20 contiguous nucleotides. Such conditions are defined supra.

The test samples suitable for nucleic acid probing methods of the present invention include, for example, cells or nucleic acid extracts of cells, or biological fluids. The samples used in the above-described methods will vary based on the assay format, the detection method and the nature of the tissues, cells or extracts to be assayed. Methods for preparing nucleic acid extracts of cells are well-known in the art and can be readily adapted in order to obtain a sample that is compatible with the method utilized.

In another aspect, the invention provides methods for detection of a polynucleotide in a sample as a diagnostic tool for diseases or disorders caused by an ectoparasite, wherein the methods comprise the steps of: (a) contacting the sample with a nucleic acid probe which hybridizes under hybridization assay conditions to a nucleic acid target region encoding a polypeptide having a sequence selected from SEQ ID NO: 2, 4, 6, 8, 10, 12, 14, 16, 18, 20, 22, and 24, said probe comprising the nucleic acid sequence encoding the polypeptide, fragments thereof, and the complements of the sequences and fragments; and (b) detecting the presence or amount of the probe:target region hybrid as an indication of the disease.

Alternatively, immunoassay kits can be provided which have containers having antibodies specific for the DmGPCR protein and optionally, containers with positive and negative controls and/or instructions.

Kits are also provided that are useful in the identification of DmGPCR binding partners, such as natural ligands or modulators (agonists or antagonists). Substances useful for treatment of disorders or diseases may show positive results in one or more in vitro assays for an activity corresponding to treatment of the disease or disorder in question. Substances that modulate the activity of the polypeptides include, but are not limited to, antisense oligonucleotides, agonists and antagonists, and antibodies.

The invention also provides methods for modulating ligand binding of a DmGPCR comprising the step of contacting the DmGPCR with an antibody specific for the DmGPCR, under conditions wherein the antibody binds the receptor.

Methods of Inducing Immune Response

Another aspect of the present invention is directed to methods of inducing an immune response in a subject against a polypeptide of the invention by administering to the subject an amount of the polypeptide sufficient to induce an immune response. The amount will be dependent on the species of the subject, size of the subject, and the like but can be determined by those skilled in the art.

Methods of Identifying Ligands

Another aspect of the present invention is directed to methods of identifying compounds that bind to either DmGPCR or nucleic acid molecules encoding DmGPCR, comprising contacting DmGPCR, or a nucleic acid molecule encoding the same, with a compound, and determining whether the compound binds DmGPCR or a nucleic acid molecule encoding the same. Binding can be determined by binding assays which are well-known to the skilled artisan, including, but not limited to, gel-shift assays, Western blots, radiolabeled competition assay, phage-based expression cloning, co-fractionation by chromatography, co-precipitation, cross linking, interaction trap/two-hybrid analysis, southwestern analysis, ELISA, and the like, which are described in, for example, CURRENT PROTOCOLS IN MOLECULAR BIOLOGY, John Wiley & Sons, NY, 1999, which is incorporated herein by reference in its entirety. The compounds to be screened include (which may include compounds which are suspected to bind DmGPCR, or a nucleic acid molecule encoding the same), but are not limited to, compounds of extracellular, intracellular, biological, or chemical origin.

The invention also provides assays to identify compounds that bind a DmGPCR. One such assay comprises contacting a composition comprising a DmGPCR with a compound suspected of binding DmGPCR and measuring binding between the compound and DmGPCR. In some embodiments, the composition comprises a cell expressing DmGPCR on its surface. In another variation, isolated DmGPCR or cell membranes comprising DmGPCR are employed. The binding may be measured directly, e.g., by using a labeled compound, or may be measured indirectly by several techniques, including measuring intracellular signaling of DmGPCR induced by the compound (or measuring changes in the level of DmGPCR signaling).

Specific binding molecules, including natural ligands and synthetic compounds, can be identified or developed using isolated or recombinant DmGPCR products, DmGPCR variants, or cells expressing such products. Binding partners are useful for purifying DmGPCR products and detection or quantification of DmGPCR products in fluid and tissue samples using known immunological procedures. Binding molecules are also manifestly useful in modulating (i.e., blocking, inhibiting, or stimulating) biological activities of DmGPCR, especially those activities involved in signal transduction.

The DNA and amino acid sequence information provided by the present invention also makes possible identification of binding partner compounds with which a DmGPCR polypeptide or polynucleotide will interact. Methods to identify binding partner compounds include solution assays, in vitro assays wherein DmGPCR polypeptides are immobilized, and cell-based assays. Identification of binding partner compounds of DmGPCR polypeptides provides candidates for therapeutic or prophylactic intervention in pathologies associated with ectoparasites expressing DmGPCR and candidates for insecticides.

The invention includes several assay systems for identifying DmGPCR binding partners. In solution assays, methods of the invention comprise the steps of (a) contacting a DmGPCR polypeptide with one or more candidate binding partner compounds and (b) identifying the compounds that bind to the DMGPCR polypeptide. Identification of the compounds that bind the DmGPCR polypeptide can be achieved by isolating the DmGPCR polypeptide/binding partner complex, and separating the binding partner compound from the DMGPCR polypeptide. An additional step of characterizing the physical, biological, and/or biochemical properties of the binding partner compound is also comprehended in another embodiment of the invention. In one aspect, the DmGPCR polypeptide/binding partner complex is isolated using an antibody immunospecific for either the DmGPCR polypeptide or the candidate binding partner compound.

In still other embodiments, either the DmGPCR polypeptide or the candidate binding partner compound comprises a label or tag that facilitates its isolation, and methods of the invention to identify binding partner compounds include a step of isolating the DmGPCR polypeptide/binding partner complex through interaction with the label or tag. An exemplary tag of this type is a poly-histidine sequence, generally around six histidine residues, that permits isolation of a compound so labeled using nickel chelation. Other labels and tags, such as the FLAG® tag (Eastman Kodak, Rochester, N.Y.), well-known and routinely used in the art, are embraced by the invention. Labels of the invention also include but are not limited to, a radiolabel (e.g., ¹²⁵I, ³⁵S, ³²P, ³³P, ³H), a fluorescence label, a chemiluminescent label, an enzymic label, and an immunogenic label.

In some embodiments of in vitro assays, the invention provides methods comprising the steps of (a) contacting an immobilized DmGPCR polypeptide with a candidate binding partner compound and (b) detecting binding of the candidate compound to the DmGPCR polypeptide. In an alternative embodiment, the candidate binding partner compound is immobilized and binding of DmGPCR is detected. Immobilization is accomplished using any of the methods well-known in the art, including covalent bonding to a support, a bead, or a chromatographic resin, as well as non-covalent, high affinity interactions such as antibody binding, or use of streptavidin/biotin binding wherein the immobilized compound includes a biotin moiety. Detection of binding can be accomplished (i) using a radioactive label on the compound that is not immobilized, (ii) using a fluorescent label on the non-immobilized compound, (iii) using an antibody immunospecific for the non-immobilized compound, (iv) using a label on the non-immobilized compound that excites a fluorescent support to which the immobilized compound is attached, as well as other techniques well-known and routinely practiced in the art.

The invention also provides cell-based assays to identify binding partner compounds of a DmGPCR polypeptide. In one embodiment, the invention provides methods comprising the steps of contacting a DmGPCR polypeptide expressed on the surface of a cell with a candidate binding partner compound and detecting binding of the candidate binding partner compound to the DmGPCR polypeptide. In another embodiment, the detection comprises detecting a calcium flux or other physiological event in the cell caused by the binding of the molecule.

In another embodiment of the invention, high throughput screening for compounds having suitable binding affinity to DmGPCR is employed. Briefly, large numbers of different small peptide test compounds are synthesized on a solid support or as free compounds dissolved in appropriate buffers. The peptide test compounds are contacted with DmGPCR and washed. Bound DmGPCR is then detected by methods well-known in the art. Purified polypeptides of the invention can also be coated directly onto plates for use in the aforementioned binding assays. In addition, non-neutralizing antibodies can be used to capture the protein and immobilize it on the solid support.

Generally, an expressed DmGPCR can be used for HTS binding assays in conjunction with its defined ligand. The identified peptide is labeled with a suitable radioisotope, including, but not limited to, ¹²⁵I, ³H, ³⁵S or ³²P, by methods that are well-known to those skilled in the art. Alternatively, the peptides may be labeled by well-known methods with a suitable fluorescent derivative (Baindur et al., Drug Dev. Res., 1994, 33, 373-398; Rogers, Drug Discovery Today, 1997, 2, 156-160). Radioactive ligand specifically bound to the receptor in membrane preparations made from the cell line expressing the recombinant protein can be detected in HTS assays in one of several standard ways, including filtration of the receptor-ligand complex to separate bound ligand from unbound ligand (Williams, Med. Res. Rev., 1991, 11, 147-184; Sweetnam et al., J. Natural Products, 1993, 56, 441-455). Alternative methods include a scintillation proximity assay (SPA) or a FlashPlate format in which such separation is unnecessary (Nakayama, Curr. Opinion Drug Disc. Dev., 1998, 1, 85-91 Bosse et al., J. Biomolecular Screening, 1998, 3, 285-292). Binding of fluorescent ligands can be detected in various ways, including fluorescence energy transfer (FRET), direct spectrophotofluorometric analysis of bound ligand, or fluorescence polarization (Rogers, Drug Discovery Today, 1997, 2, 156-160; Hill, Curr. Opinion Drug Disc. Dev., 1998, 1, 92-97).

Other assays may be used to identify specific ligands of a DmGPCR, including assays that identify ligands of the target protein through measuring direct binding of test ligands to the target protein, as well as assays that identify ligands of target proteins through affinity ultrafiltration with ion spray mass spectroscopy/HPLC methods or other physical and analytical methods. Alternatively, such binding interactions are evaluated indirectly using the yeast two-hybrid system described in Fields et al. (Nature, 1989, 340, 245-246) and Fields et al. (Trends in Genetics, 1994, 10, 286-292), both of which are incorporated herein by reference. The two-hybrid system is a genetic assay for detecting interactions between two proteins or polypeptides. It can be used to identify proteins that bind to a known protein of interest, or to delineate domains or residues critical for an interaction. Variations on this methodology have been developed to clone genes that encode DNA binding proteins, to identify peptides that bind to a protein, and to screen for drugs. The two-hybrid system exploits the ability of a pair of interacting proteins to bring a transcription activation domain into close proximity with a DNA binding domain that binds to an upstream activation sequence (UAS) of a reporter gene, and is generally performed in yeast. The assay requires the construction of two hybrid genes encoding (1) a DNA-binding domain that is fused to a first protein and (2) an activation domain fused to a second protein. The DNA-binding domain targets the first hybrid protein to the UAS of the reporter gene; however, because most proteins lack an activation domain, this DNA-binding hybrid protein does not activate transcription of the reporter gene. The second hybrid protein, which contains the activation domain, cannot by itself activate expression of the reporter gene because it does not bind the UAS. However, when both hybrid proteins are present, the noncovalent interaction of the first and second proteins tethers the activation domain to the UAS, activating transcription of the reporter gene. For example, when the first protein is a DmGPCR gene product, or fragment thereof, that is known to interact with another protein or nucleic acid, this assay can be used to detect agents that interfere with the binding interaction. Expression of the reporter gene is monitored as different test agents are added to the system. The presence of an inhibitory agent results in lack of a reporter signal.

When the function of the DmGPCR gene product is unknown and no ligands are known to bind the gene product, the yeast two-hybrid assay can also be used to identify proteins that bind to the gene product. In an assay to identify proteins that bind to a DmGPCR receptor, or fragment thereof, a fusion polynucleotide encoding both a DmGPCR receptor (or fragment) and a UAS binding domain (i.e., a first protein) may be used. In addition, a large number of hybrid genes each encoding a different second protein fused to an activation domain are produced and screened in the assay. Typically, the second protein is encoded by one or more members of a total cDNA or genomic DNA fusion library, with each second protein coding region being fused to the activation domain. This system is applicable to a wide variety of proteins, and it is not even necessary to know the identity or function of the second binding protein. The system is highly sensitive and can detect interactions not revealed by other methods; even transient interactions may trigger transcription to produce a stable mRNA that can be repeatedly translated to yield the reporter protein.

Other assays may be used to search for agents that bind to the target protein. One such screening method to identify direct binding of test ligands to a target protein is described in U.S. Pat. No. 5,585,277, incorporated herein by reference. This method relies on the principle that proteins generally exist as a mixture of folded and unfolded states, and continually alternate between the two states. When a test ligand binds to the folded form of a target protein (i.e., when the test ligand is a ligand of the target protein), the target protein molecule bound by the ligand remains in its folded state. Thus, the folded target protein is present to a greater extent in the presence of a test ligand which binds the target protein, than in the absence of a ligand. Binding of the ligand to the target protein can be determined by any method that distinguishes between the folded and unfolded states of the target protein. The function of the target protein need not be known in order for this assay to be performed. Virtually any agent can be assessed by this method as a test ligand, including, but not limited to, metals, polypeptides, proteins, lipids, polysaccharides, polynucleotides, and small organic molecules.

Another method for identifying ligands of a target protein is described in Wieboldt et al. (Anal. Chem., 1997, 69, 1683-1691), incorporated herein by reference. This technique screens combinatorial libraries of 20-30 agents at a time in solution phase for binding to the target protein. Agents that bind to the target protein are separated from other library components by simple membrane washing. The specifically selected molecules that are retained on the filter are subsequently liberated from the target protein and analyzed by HPLC and pneumatically assisted electrospray (ion spray) ionization mass spectroscopy. This procedure selects library components with the greatest affinity for the target protein, and is particularly useful for small molecule libraries.

Other embodiments of the invention comprise using competitive screening assays in which neutralizing antibodies capable of binding a polypeptide of the invention specifically compete with a test compound for binding to the polypeptide. In this manner, the antibodies can be used to detect the presence of any peptide that shares one or more antigenic determinants with DmGPCR. Radiolabeled competitive binding studies are described in A. H. Lin et al. (Antimicrobial Agents and Chemotherapy, 1997, 41(10), 2127-2131), the disclosure of which is incorporated herein by reference in its entirety.

Methods for Identifying Modulating Agents

The invention also provides methods for identifying a modulator of binding between a DmGPCR and a DmGPCR binding partner, comprising the steps of: (a) contacting a DmGPCR binding partner and a composition comprising a DmGPCR in the presence and in the absence of a putative modulator compound; (b) detecting binding between the binding partner and the DmGPCR; and (c) identifying a putative modulator compound or a modulator compound in view of decreased or increased binding between the binding partner and the DmGPCR in the presence of the putative modulator, as compared to binding in the absence of the putative modulator.

DmGPCR binding partners that stimulate DmGPCR activity are useful as agonists in conditions characterized by insufficient DmGPCR signaling (e.g., as a result of insufficient activity of a DmGPCR ligand). DmGPCR binding partners that block ligand-mediated DmGPCR signaling are useful as DmGPCR antagonists in conditions characterized by excessive DmGPCR signaling. In addition, DmGPCR modulators in general, as well as DmGPCR polynucleotides and polypeptides, are useful in diagnostic assays for diseases caused by ectoparasites or conditions in which DmGPCR activity is enhanced or impaired.

In another aspect, the invention provides methods for treating a disease or condition by administering to a subject in need of such treatment a substance that modulates the activity or expression of a polypeptide having a sequence selected from SEQ ID NOs: 2, 4, 6, 8, 10, 12, 14, 16, 18, 20, 22, and 24.

In another aspect, the invention provides methods for controlling an insect population by administering to an insect population a binding partner or modulator that modifies expression or activity of a DmGPCR.

Agents that modulate (i.e., increase, decrease, or block) DmGPCR activity or expression may be identified by incubating a putative modulator with a cell containing a DmGPCR polypeptide or polynucleotide and determining the effect of the putative modulator on DmGPCR activity or expression. The selectivity of a compound that modulates the activity of DmGPCR can be evaluated by comparing its effects on DmGPCR to its effect on other GPCR compounds. Selective modulators may include, for example, antibodies and other proteins, peptides, or organic molecules which specifically bind to a DmGPCR polypeptide or a DmGPCR-encoding nucleic acid. Modulators of DmGPCR activity will be therapeutically useful in treatment of diseases and physiological conditions in which normal or aberrant DmGPCR activity is involved.

DmGPCR polynucleotides and polypeptides, as well as DmGPCR modulators, may also be used in diagnostic assays for diseases caused by ectoparasites or conditions characterized by enhanced or impaired DmGPCR activity.

Methods of the invention to identify modulators include variations on any of the methods described above to identify binding partner compounds, the variations including techniques wherein a binding partner compound has been identified and the binding assay is carried out in the presence and absence of a candidate modulator. A modulator is identified in those instances where binding between the DmGPCR polypeptide and the binding partner compound changes in the presence of the candidate modulator compared to binding in the absence of the candidate modulator compound. A modulator that increases binding between the DmGPCR polypeptide and the binding partner compound is described as an enhancer or activator, and a modulator that decreases binding between the DmGPCR polypeptide and the binding partner compound is described as an inhibitor.

The invention also comprehends high-throughput screening (HTS) assays to identify compounds that interact with or inhibit biological activity (i.e., affect enzymatic activity, binding activity, etc.) of a DmGPCR polypeptide. HTS assays permit screening of large numbers of compounds in an efficient manner. Cell-based HTS systems are contemplated to investigate DmGPCR receptor-ligand interaction. HTS assays are designed to identify “hits” or “lead compounds” having the desired property, from which modifications can be designed to improve the desired property. Chemical modification of the “hit” or “lead compound” is often based on an identifiable structure/activity relationship between the “hit” and the DmGPCR polypeptide.

Modulators falling within the scope of the invention include, but are not limited to, non-peptide molecules such as non-peptide mimetics, non-peptide allosteric effectors, and peptides. The DmGPCR polypeptide or polynucleotide employed in such a test may either be free in solution, attached to a solid support, borne on a cell surface or located intracellularly, or associated with a portion of a cell. One skilled in the art can, for example, measure the formation of complexes between DmGPCR and the compound being tested. Alternatively, one skilled in the art can examine the diminution in complex formation between DmGPCR and its substrate caused by the compound being tested.

Another aspect of the present invention is directed to methods of identifying compounds which modulate (i.e., increase or decrease) activity of DmGPCR comprising contacting DmGPCR with a compound, and determining whether the compound modifies activity of DmGPCR. The activity in the presence of the test compound is compared to the activity in the absence of the test compound. Where the activity of the sample containing the test compound is higher than the activity in the sample lacking the test compound, the compound will have increased activity. Similarly, where the activity of the sample containing the test compound is lower than the activity in the sample lacking the test compound, the compound will have inhibited activity.

The present invention is particularly useful for screening compounds by using DmGPCR in any of a variety of activity assays. The compounds to be screened include (which may include compounds which are suspected to modulate DmGPCR activity), but are not limited to, compounds of extracellular, intracellular, biological, or chemical origin. The DmGPCR polypeptide employed in such a test may be in any form, such as free in solution, attached to a solid support, borne on a cell surface, or located intracellularly. One skilled in the art can, for example, measure the formation of complexes between DmGPCR and the compound being tested. Alternatively, one skilled in the art can examine the diminution in complex formation between DmGPCR and its substrate caused by the compound being tested.

The activity of DmGPCR polypeptides of the invention can be determined by, for example, examining the ability to bind or be activated by chemically synthesized peptide ligands. Alternatively, the activity of the DmGPCRs can be assayed by examining their ability to bind calcium ions, hormones, chemokines, neuropeptides, neurotransmitters, nucleotides, lipids, odorants, and photons. Alternatively, the activity of the DmGPCRs can be determined by examining the activity of effector molecules including, but not limited to, adenylate cyclase, phospholipases, and ion channels. Thus, modulators of DmGPCR activity may alter a DmGPCR receptor function, such as a binding property of a receptor or an activity such as G protein-mediated signal transduction or membrane localization. In various embodiments of the methods, the assay may take the form of an ion flux assay, a yeast growth assay, a non-hydrolyzable GTP assay such as a [³⁵S]GTPγS assay, a cAMP assay, an inositol triphosphate assay, a diacylglycerol assay, an Aequorin assay, a Luciferase assay, a FLIPR assay for intracellular Ca²⁺ concentration, a mitogenesis assay, a MAP Kinase activity assay, an arachidonic acid release assay (e.g., using [³H]-arachidonic acid), and an assay for extracellular acidification rates, as well as other binding or function-based assays of DmGPCR activity that are generally known in the art. In several of these embodiments, the invention comprehends the inclusion of any of the G proteins known in the art, such as G₁₆, G₁₅, or chimeric G_{qi5}, G_{qi5}, G_{qs5}, G_{qz5}, and the like. DmGPCR activity can be determined by methodologies that are used to assay for FaRP activity, which is well-known to those skilled in the art. Biological activities of DmGPCR receptors according to the invention include, but are not limited to, the binding of a natural or an unnatural ligand, as well as any one of the functional activities of GPCRs known in the art. Non-limiting examples of GPCR activities include transmembrane signaling of various forms, which may involve G protein association and/or the exertion of an influence over G protein binding of various guanidylate nucleotides; another exemplary activity of GPCRs is the binding of accessory proteins or polypeptides that differ from known G proteins.

The modulators of the invention exhibit a variety of chemical structures, which can be generally grouped into non-peptide mimetics of natural DmGPCR receptor ligands, peptide, and non-peptide allosteric effectors of DmGPCR receptors, and peptides that may function as activators or inhibitors (competitive, uncompetitive and non-competitive) (e.g., antibody products) of DmGPCR receptors. The invention does not restrict the sources for suitable modulators, which may be obtained from natural sources such as plant, animal or mineral extracts, or non-natural sources such as small molecule libraries, including the products of combinatorial chemical approaches to library construction, and peptide libraries. Examples of peptide modulators of DmGPCR receptors exhibit the following primary structures: GLGPRPLRFamide (SEQ ID NO: 49), GNSFLRFamide (SEQ ID NO: 136), GGPQGPLRFamide (SEQ ID NO: 102), GPSGPLRFamide (SEQ ID NO: 103), PDVDHVFLRFamide (SEQ ID NO: 150), and pyro-EDVDHVFLRFamide (SEQ ID NO: 167).

Other assays can be used to examine enzymatic activity including, but not limited to, photometric, radiometric, HPLC, electrochemical, and the like, which are described in, for example, ENZYME ASSAYS: A PRACTICAL APPROACH, eds. R. Eisenthal and M. J. Danson, 1992, Oxford University Press, which is incorporated herein by reference in its entirety.

The use of cDNAs encoding GPCRs in activity assays is well-known; assays capable of testing thousands of unknown compounds per day in high-throughput screens (HTSs) are thoroughly documented. The literature is replete with examples of the use of radiolabelled ligands in HTS binding assays for drug discovery (see Williams, Medicinal Research Reviews, 1991, 11, 147-184; Sweetnam, et al., J. Natural Products, 1993, 56, 441-455 for review). Recombinant receptors are preferred for binding assay HTS because they allow for better specificity (higher relative purity), provide the ability to generate large amounts of receptor material, and can be used in a broad variety of formats (see Hodgson, Bio/Technology, 1992, 10, 973-980, incorporated herein by reference in its entirety).

A variety of heterologous systems are available for functional expression of recombinant receptors that are well-known to those skilled in the art. Such systems include bacteria (Strosberg, et al., Trends in Pharmacological Sciences, 1992, 13, 95-98), yeast (Pausch, Trends in Biotechnology, 1997, 15, 487-494), several kinds of insect cells (Vanden Broeck, Int. Rev. Cytology, 1996, 164, 189-268), amphibian cells (Jayawickreme et al., Curr. Opin. Biotechnol., 1997, 8, 629-634) and several mammalian cell lines (CHO, HEK293, COS, etc.; see Gerhardt, et al., Eur. J. Pharmacology, 1997, 334, 1-23). These examples do not preclude the use of other possible cell expression systems, including cell lines obtained from nematodes (PCT application WO 98/37177).

In some embodiments of the invention, methods of screening for compounds which modulate DmGPCR activity comprise contacting test compounds with DmGPCR and assaying for the presence of a complex between the compound and DmGPCR. In such assays, the ligand is typically labeled. After suitable incubation, free ligand is separated from that present in bound form, and the amount of free or uncomplexed label is a measure of the ability of the particular compound to bind to DmGPCR.

It is well-known that activation of heterologous receptors expressed in recombinant systems results in a variety of biological responses, which are mediated by G proteins expressed in the host cells. Occupation of a GPCR by an agonist results in exchange of bound GDP for GTP at a binding site on the G_{α} subunit; one can use a radioactive, non-hydrolyzable derivative of GTP, [³⁵S]GTPγS, to measure binding of an agonist to the receptor (Sim et al., Neuroreport, 1996, 7, 729-733). One can also use this binding to measure the ability of antagonists to bind to the receptor by decreasing binding of [³⁵S]GTPγS in the presence of a known agonist. One could therefore construct a HTS assay based on [³⁵S]GTPγS binding.

The G proteins required for functional expression of heterologous GPCRs can be native constituents of the host cell or can be introduced through well-known recombinant technology. The G proteins can be intact or chimeric. Often, a nearly universally competent G protein (e.g., G_{α16}) is used to couple any given receptor to a detectable response pathway. G protein activation results in the stimulation or inhibition of other native proteins, events that can be linked to a measurable response.

Examples of such biological responses include, but are not limited to, the following: the ability to survive in the absence of a limiting nutrient in specifically engineered yeast cells (Pausch, Trends in Biotechnology, 1997, 15, 487-494); changes in intracellular Ca²⁺ concentration as measured by fluorescent dyes (Murphy, et al., Curr. Opin. Drug Disc. Dev., 1998, 1, 192-199). Fluorescence changes can also be used to monitor ligand-induced changes in membrane potential or intracellular pH; an automated system suitable for HTS has been described for these purposes (Schroeder, et al., J. Biomolecular Screening, 1996, 1, 75-80). Melanophores prepared from Xenopus laevis show a ligand-dependent change in pigment organization in response to heterologous GPCR activation; this response is adaptable to HTS formats (Jayawickreme, et al., Curr. Opin. Biotechnol., 1997, 8, 629-634). Assays are also available for the measurement of common second messengers, including cAMP, phosphoinositides, and arachidonic acid.

Methods of HTS employing these receptors include permanently transfected CHO cells, in which agonists and antagonists can be identified by the ability to specifically alter the binding of [³⁵S]GTPγS in membranes prepared from these cells. In another embodiment of the invention, permanently transfected CHO cells could be used for the preparation of membranes which contain significant amounts of the recombinant receptor proteins; these membrane preparations would then be used in receptor binding assays, employing the radiolabelled ligand specific for the particular receptor. Alternatively, a functional assay, such as fluorescent monitoring of ligand-induced changes in internal Ca²⁺ concentration or membrane potential in permanently transfected CHO cells containing each of these receptors individually or in combination would be useful for HTS. Equally useful would be an alternative type of mammalian cell, such as HEK293 or COS cells, in similar formats. Permanently transfected insect cell lines, such as Drosophila S2 cells, and recombinant yeast cells expressing the Drosophila melanogaster receptors in HTS formats well-known to those skilled in the art (e.g., Pausch, Trends in Biotechnology, 1997, 15, 487-494), would also be useful in the invention.

The invention contemplates a multitude of assays to screen and identify inhibitors of ligand binding to DmGPCR receptors. In one example, the DmGPCR receptor is immobilized and interaction with a binding partner is assessed in the presence and absence of a candidate modulator such as an inhibitor compound. In another example, interaction between the DmGPCR receptor and its binding partner is assessed in a solution assay, both in the presence and absence of a candidate inhibitor compound. In either assay, an inhibitor is identified as a compound that decreases binding between the DmGPCR receptor and its binding partner. Another contemplated assay involves a variation of the di-hybrid assay wherein an inhibitor of protein/protein interactions is identified by detection of a positive signal in a transformed or transfected host cell, as described in PCT publication number WO 95/20652, published Aug. 3, 1995.

Candidate modulators contemplated by the invention include compounds selected from libraries of either potential activators or potential inhibitors. There are a number of different libraries used for the identification of small molecule modulators, including: (1) chemical libraries, (2) natural product libraries, and (3) combinatorial libraries comprised of random peptides, oligonucleotides, or organic molecules. Chemical libraries consist of random chemical structures, some of which are analogs of known compounds or analogs of compounds that have been identified as “hits” or “leads” in other drug discovery screens, some of which are derived from natural products, and some of which arise from non-directed synthetic organic chemistry. Natural product libraries are collections of microorganisms, animals, plants, or marine organisms which are used to create mixtures for screening by: (1) fermentation and extraction of broths from soil, plant or marine microorganisms or (2) extraction of plants or marine organisms. Natural product libraries include polyketides, non-ribosomal peptides, and variants (non-naturally occurring) thereof. For a review, see Science, 1998, 282, 63-68. Combinatorial libraries are composed of large numbers of peptides, oligonucleotides, or organic compounds as a mixture. These libraries are relatively easy to prepare by traditional automated synthesis methods, PCR, cloning, or proprietary synthetic methods. Of particular interest are non-peptide combinatorial libraries. Still other libraries of interest include peptide, protein, peptidomimetic, multiparallel synthetic collection, recombinatorial, and polypeptide libraries. For a review of combinatorial chemistry and libraries created therefrom, see Myers, Curr. Opin. Biotechnol., 1997, 8, 701-707. Identification of modulators through use of the various libraries described herein permits modification of the candidate “hit” (or “lead”) to optimize the capacity of the “hit” to modulate activity.

Still other candidate inhibitors contemplated by the invention can be designed and include soluble forms of binding partners, as well as such binding partners as chimeric, or fusion, proteins. A “binding partner” as used herein broadly encompasses non-peptide modulators, as well as such peptide modulators as neuropeptides other than natural ligands, antibodies, antibody fragments, and modified compounds comprising antibody domains that are immunospecific for the expression product of the identified DmGPCR gene.

In other embodiments of the invention, the polypeptides of the invention are employed as a research tool for identification, characterization and purification of interacting, regulatory proteins. Appropriate labels are incorporated into the polypeptides of the invention by various methods known in the art and the polypeptides are used to capture interacting molecules. For example, molecules are incubated with the labeled polypeptides, washed to remove unbound polypeptides, and the polypeptide complex is quantified. Data obtained using different concentrations of polypeptide are used to calculate values for the number, affinity, and association of polypeptide with the protein complex.

Labeled polypeptides are also useful as reagents for the purification of molecules with which the polypeptide interacts including, but not limited to, inhibitors. In one embodiment of affinity purification, a polypeptide is covalently coupled to a chromatography column. Cells and their membranes are extracted, and various cellular subcomponents are passed over the column. Molecules bind to the column by virtue of their affinity to the polypeptide. The polypeptide-complex is recovered from the column, dissociated and the recovered molecule is subjected to protein sequencing. This amino acid sequence is then used to identify the captured molecule or to design degenerate oligonucleotides for cloning the corresponding gene from an appropriate cDNA library.

Alternatively, compounds may be identified which exhibit similar properties to the ligand for the DmGPCR of the invention, but which are smaller and exhibit a longer half-life than the endogenous ligand in a human or animal body. When an organic compound is designed, a molecule according to the invention is used as a “lead” compound. The design of mimetics to known pharmaceutically active compounds is a well-known approach in the development of pharmaceuticals based on such “lead” compounds. Mimetic design, synthesis, and testing are generally used to avoid randomly screening a large number of molecules for a target property. Furthermore, structural data deriving from the analysis of the deduced amino acid sequences encoded by the DNAs of the present invention are useful to design new drugs which are more specific and, therefore, have a higher pharmacological potency.

Comparison of the protein sequences of the present invention with the sequences present in all the available databases showed a significant homology with the transmembrane portion of G protein coupled receptors. Accordingly, computer modelling can be used to develop a putative tertiary structure of the proteins of the invention based on the available information of the transmembrane domain of other proteins. Thus, novel ligands based on the predicted structure of DmGPCR can be designed.

In a particular embodiment, the novel molecules identified by the screening methods according to the invention are low molecular weight organic molecules, in which case a composition or pharmaceutical composition can be prepared thereof for oral intake, such as in tablets. The compositions, or pharmaceutical compositions, comprising the nucleic acid molecules, vectors, polypeptides, antibodies and compounds identified by the screening methods described herein, may be prepared for any route of administration including, but not limited to, oral, intravenous, cutaneous, subcutaneous, nasal, intramuscular, or intraperitoneal. The nature of the carrier or other ingredients will depend on the specific route of administration and particular embodiment of the invention to be administered. Examples of techniques and protocols that are useful in this context are, inter alia, found in REMINGTON'S PHARMACEUTICAL SCIENCES, Osol, A (ed.), 1980, which is incorporated herein by reference in its entirety.

The dosage of these low molecular weight compounds will depend on the disease state or condition to be treated and other clinical factors, such as weight and condition of the subject to be treated and the route of administration of the compound. For treating animals, between approximately 0.5 mg/kg of body weight to 500 mg/kg of body weight of the compound can be administered. Therapy is typically administered at lower dosages and is continued until the desired therapeutic outcome is observed.

Methods of determining the dosages of compounds to be administered to a subject and modes of administering compounds to an organism are disclosed in U.S. application Ser. No. 08/702,282, filed Aug. 23, 1996 and International patent publication number WO 96/22976, published Aug. 1, 1996, both of which are incorporated herein by reference in their entirety, including any drawings, figures or tables. Those skilled in the art will appreciate that such descriptions are applicable to the present invention and can be easily adapted to it.

The proper dosage depends on various factors such as the type of disease being treated, the particular composition being used, and the size and physiological condition of the subject. Therapeutically effective doses for the compounds described herein can be estimated initially from cell culture and animal models. For example, a dose can be formulated in animal models to achieve a circulating concentration range that initially takes into account the IC₅₀ as determined in cell culture assays.

Plasma half-life and biodistribution of the drug and metabolites in the plasma, tumors, and major organs can also be determined to facilitate the selection of drugs most appropriate to inhibit a disorder. Such measurements can be carried out. For example, HPLC analysis can be performed on the plasma of animals treated with the drug and the location of radiolabeled compounds can be determined using detection methods such as X-ray, CAT scan and MRI. Compounds that show potent inhibitory activity in the screening assays, but have poor pharmacokinetic characteristics, can be optimized by altering the chemical structure and retesting. In this regard, compounds displaying good pharmaco-kinetic characteristics can be used as a model.

Toxicity studies can also be carried out by measuring the blood cell composition. For example, toxicity studies can be carried out in a suitable animal model as follows: 1) the compound is administered to mice (an untreated control mouse should also be used); 2) blood samples are periodically obtained via the tail vein from one mouse in each treatment group; and 3) the samples are analyzed for red and white blood cell counts, blood cell composition and the percent of lymphocytes versus polymorphonuclear cells. A comparison of results for each dosing regime with the controls indicates if toxicity is present.

At the termination of each toxicity study, further studies can be carried out by sacrificing the animals (preferably, in accordance with the American Veterinary Medical Association guidelines Report of the American Veterinary Medical Assoc. Panel on Euthanasia, J. Amer. Vet. Med. Assoc., 1993, 202, 229-249). Representative animals from each treatment group can then be examined by gross necropsy for immediate evidence of metastasis, unusual illness, or toxicity. Gross abnormalities in tissue are noted and tissues are examined histologically.

The present compounds and methods, including nucleic acid molecules, polypeptides, antibodies, compounds identified by the screening methods described herein, have a variety of pharmaceutical and agricultural (e.g., insecticidal) applications and may be used, for example, to treat or prevent conditions caused by ectoparasites or to control an insect population.

The present invention also encompasses methods of agonizing (stimulating) or antagonizing a DmGPCR natural binding partner associated activity in a subject comprising administering to said subject an agonist or antagonist to one of the above disclosed polypeptides in an amount sufficient to effect said agonism or antagonism. One embodiment of the present invention, then, is a method of treating diseases or conditions in a subject caused by an ectoparasite with an agonist or antagonist of the protein of the present invention comprising administering the agonist or antagonist to a subject in an amount sufficient to agonize or antagonize the ectoparasitic-DmGPCR-associated functions.

The following Table 4 contains the sequences of the polynucleotides and polypeptides of the invention.

TABLE 4

The following DNA sequence for DmGPCR1 (SEQ ID NO:1) was identified in
D. melanogaster:
ATGGCCAACTTAAGCTGGCTGAGCACCATCACCACCACCTCCTCCTCCATCAGCACCAGC
CAGCTGCCATTGGTCAGCACAACCAACTGGAGCCTAACGTCGCCGGGAACTACTAGCGCT
ATCTTGGCGGATGTGGCTGCATCGGATGAGGATAGGAGCGGCGGGATCATTCACAACCAG
TTCGTGCAAATCTTCTTCTACGTCCTGTACGCCACGGTCTTTGTCCTGGGTGTCTTCGGA
AATGTCCTGGTTTGCTACGTAGTTCTGAGGAATCGGGCCATGCAGACTGTGACCAATATA
TTCATCACGAATCTGGCCCTGTCGGACATATTGCTCTGCGTCCTGGCGGTGCCATTTACT
CCGCTTTACACGTTCATGGGTCGCTGGGCCTTCGGCAGGAGTCTGTGCCATCTGGTGTCC
TTTGCCCAGGGATGCAGCATCTACATATCCACGCTGACCCTCACCTCGATTGCCATCGAT
CGGTACTTCGTTATCATATACCCCTTCCATCCGCGCATGAAGCTCTCCACCTGCATCGGG
ATCATAGTGAGCATCTGGGTGATAGCCCTGCTGGCCACCGTTCCCTACGGCATGTACATG
AAGATGACCAACGAGCTGGTGAACGGAACGCAGACAGGCAACGAGACCCTGGTGGAGGCC
ACTCTAATGCTAAACGGAAGCTTTGTGGCCCAGGGATCAGGATTCATCGAGGCGCCGGAC
TCTACCTCGGCCACCCAGGCCTATATGCAGGTGATGACCGCCGGATCAACGGGACCGGAG
ATGCCCTATGTGCGGGTGTACTGCGAGGAGAACTGGCCATCGGAGCAGTACCGGAAGGTG
TTCGGTGCCATCACAACCACTCTGCAGTTTGTGCTGCCCTTCTTCATCATCTCGATTTGC
TACGTGTGGATATCGGTGAAGCTAAACCAGCGGGCCAGGGCCAAGCCGGGATCGAAATCC
TCGAGACGGGAGGAGGCGGATCGGGATCGCAAGAAGCGCACCAACCGCATGCTCATCGCC
ATGGTGGCGGTATTCGGACTCAGCTGGCTGCCCATCAATGTGGTCAACATATTCGATGAC
TTCGATGACAAGTCCAACGAGTGGCGCTTCTACATCCTATTCTTCTTTGTGGCCCACTCT
ATTGCCATGAGCTCCACCTGCTACAATCCCTTCCTGTACGCCTGGCTGAACGAGAACTTC
CGCAAGGAGTTCAAGCACGTGCTGCCCTGCTTTAATCCCTCGAACAACAACATCATCAAC
ATCACCAGGGGCTATAATCGGAGTGATCGGAACACCTGTGGTCCGCGACTGCATCATGGC
AAGGGGGATGGTGGCATGGGCGGTGGCAGTCTGGACGCCGACGACCAGGACGAGAACGGC
ATCACCCAGGAGACCTGTCTGCCCAAGGAGAAGCTGCTGATTATCCCCAGGGAGCCGACT
TACGGCAATGGCACGGGTGCCGTGTCGCCAATCCTTAGCGGGCGCGGCATTAACGCCGCC
CTGGTGCACGGTGGCGACCATCAGATGCACCAGCTGCAGCCGTCACACCATCAACAGGTG
GAGCTGACGAGGCGAATCCGCCGGCGGACAGACGAGACGGACGGGGATTACCTGGACTCC
GGCGACGAGCAGACCGTGGAGGTGCGCTTCAGCGAGACGCCGTTCGTCAGCACGGATAAT
ACCACCGGGATCAGCATTCTGGAGACGAGTACGAGTCACTGCCAGGACTCGGATGTGATG
GTCGAGCTGGGCGAGGCAATCGGCGCCGGTGGTGGGGCAGAGCTGGGGAGGCGAATCAAC
TGA
The following amino acid sequence (SEQ ID NO:2) is the amino acid se-
quence for the protein encoded by
the DNA sequence of SEQ ID NO:1:
MANLSWLSTITTTSSSISTSQLPLVSTTNWSLTSPGTTSAILADVAASDEDRSGGIIHNQ
FVQIFFYVLYATVFVLGVFGNVLVCYVVLRNRAMQTVTNIFITNLALSDILLCVLAVPFT
PLYTFMGRWAFGRSLCHLVSFAQGCSIYISTLTLTSIAIDRYFVIIYPFHPRMKLSTCIG
IIVSIWVIALLATVPYGMYMKMTNELVNGTQTGNETLVEATLMLNGSFVAQGSGFIEAPD
STSATQAYMQVMTAGSTGPEMPYVRVYCEENWPSEQYRKVFGAITTTLQFVLPFFIISIC
YVWISVKLNQRARAKPGSKSSRREEADRDRKKRTNRMLIAMVAVFGLSWLPINVVNIFDD
FDDKSNEWRFYILFFFVAHSIAMSSTCYNPFLYAWLNENFRKEFKHVLPCFNPSNNNIIN
ITRGYNRSDRNTCGPRLHHGKGDGGMGGGSLDADDQDENGITQETCLPKEKLLIIPREPT
YGNGTGAVSPILSGRGINAALVHGGDHQMHQLQPSHHQQVELTRRIRRRTDETDGDYLDS
GDEQTVEVRFSETPFVSTDNTTGISILETSTSHCQDSDVMVELGEAIGAGGGAELGRRIN
The following DNA sequence for DmGPCR2a (SEQ ID NO:3) was identified in
D. melanogaster:
ATGAATCAGACGGAGCCCGCCCAGCTGGCAGATGGGGAGCATCTGAGTGG
ATACGCCAGCAGCAGCAACAGCGTGCGCTATCTGGACGACCGGCATCCGC
TGGACTACCTTGACCTGGGCACGGTGCACGCCCTCAACACCACTGCCATC
AACACCTCGGATCTGAATGAGACTGGGAGCAGGCCGCTGGACCCGGTGCT
TATCGATAGGTTCCTGAGCAACAGGGCGGTGGACAGCCCCTGGTACCACA
TGCTCATCAGCATGTACGGCGTGCTAATCGTCTTCGGCGCCCTAGGCAAC
ACCCTGGTTGTTATAGCCGTCATCCGGAAGCCCATCATGCGCACTGCTCG
CAATCTGTTCATCCTCAACCTGGCCATATCGGACCTACTTTTATGCCTAG
TCACCATGCCGCTGACCTTGATGGAGATCCTGTCCAAGTACTGGCCCTAC
GGCTCCTGCTCCATCCTGTGCAAAACGATTGCCATGCTGCAGGCACTTTG
TATTTTCGTGTCGACAATATCCATAACGGCCATTGCCTTCGACAGATATC
AGGTGATCGTGTACCCCACGCGGGACAGCCTGCAGTTCGTGGGCGCGGTG
ACGATCCTGGCGGGGATCTGGGCACTGGCACTGCTGCTGGCCTCGCCGCT
GTTCGTCTACAAGGAGCTGATCAACACAGACACGCCGGCACTCCTGCAGC
AGATCGGCCTGCAGGACACGATCCCGTACTGCATTGAGGACTGGCCAAGT
CGCAACGGGCGCTTCTACTACTCGATCTTCTCGCTGTGCGTACAATACCT
GGTGCCCATCCTGATCGTCTCGGTGGCATACTTCGGGATCTACAACAAGC
TGAAGAGCCGCATCACCGTGGTGGCTGTGCAGGCGTCCTCCGCTCAGCGG
AAGGTGGAGCGGGGGCGGCGGATGAAGCGCACCAACTGCCTACTGATCAG
CATCGCCATCATCTTTGGCGTTTCTTGGCTGCCGCTGAACTTTTTCAACC
TGTACGCGGACATGGAGCGCTCGCCGGTCACTCAGAGCATGCTAGTCCGC
TACGCCATCTGCCACATGATCGGCATGAGCTCCGCCTGCTCCAACCCGTT
GCTCTACGGCTGGCTCAACGACAACTTCCGTAAAGAATTTCAAGAACTGC
TCTGCCGTTGCTCAGACACTAATGTTGCTCTTAACGGTCACACGACAGGC
TGCAACGTCCAGGCGGCGGCGCGCAAGCGTCGCAAGTTGGGCGCCGAACT
CTCCAAAGGCGAACTCAAGCTGCTGGGGCCAGGCGGCGCCCAGAGCGGTA
CCGCCGGCGGGGAAGGCGGTCTGGCGGCCACCGACTTCATGACCGGCCAC
CACGAGGGCGGACTGCGCAGCGCCATAACCGAGTCGGTGGCCCTCACGGA
CCACAACCCCGTGCCCTCGGAGGTCACCAAGCTGATGCCGCGGTA
The following amino acid sequence (SEQ ID NO:4) is the amino acid se-
quence for the protein encoded by
the DNA sequence of SEQ ID NO:3:
MENTTMLANISLNATRNEENITSFFTDEEWLAINGTLPWIVGFFFGVIAITGFFGNLLVILVVVFNNNMRS
TTNLMIVNLAAADLMFVILCIPFTATDYMVYYWPYGRFWCRSVQYLIVVTAFASIYTLVLMSIDRFLAVVH
PIRSRMMRTENITLIAIVTLWIVVLVVSVPVAFTHDVVVDYDAKKNITYGMCTFTTNDFLGPRTYQVTFFI
SSYLLPLMIISGLYMRMIMRLWRQGTGVRMSKESQRGRKRVTRLVVVVVIAFASLWLPVQLILLLKSLDVI
ETNTLTKLVIQVTAQTLAYSSSCINPLLYAFLSENFRKAFYKAVNCSSRYQNYTSDLPPPRKTSCARTSTT
GL
The following DNA sequence for DmGPCR2b (SEQ ID NO:5) was identified in
D. melanogaster:
ATGAATCAGACGGAGCCCGCCCAGCTGGCAGATGGGGAGCATCTGAGTGG
ATACGCCAGCAGCAGCAACAGCGTGCGCTATCTGGACGACCGGCATCCGC
TGGACTACCTTGACCTGGGCACGGTGCACGCCCTCAACACCACTGCCATC
AACACCTCGGATCTGAATGAGACTGGGAGCAGGCCGCTGGACCCGGTGCT
TATCGATAGGTTCCTGAGCAACAGGGCGGTGGACAGCCCCTGGTACCACA
TGCTCATCAGCATGTACGGCGTGCTAATCGTCTTCGGCGCCCTAGGCAAC
ACCCTGGTTGTTATAGCCGTCATCCGGAAGCCCATCATGCGCACTGCTCG
CAATCTGTTCATCCTCAACCTGGCCATATCGGACCTACTTTTATGCCTAG
TCACCATGCCGCTGACCTTGATGGAGATCCTGTCCAAGTACTGGCCCTAC
GGCTCCTGCTCCATCCTGTGCAAAACGATTGCCATGCTGCAGGCACTTTG
TATTTTCGTGTCGACAATATCCATAACGGCCATTGCCTTCGACAGATATC
AGGTGATCGTGTACCCCACGCGGGACAGCCTGCAGTTCGTGGGCGCGGTG
ACGATCCTGGCGGGGATCTGGGCACTGGCACTGCTGCTGGCCTCGCCGCT
GTTCGTCTACAAGGAGCTGATCAACACAGACACGCCGGCACTCCTGCAGC
AGATCGGCCTGCAGGACACGATCCCGTACTGCATTGAGGACTGGCCAAGT
CGCAACGGGCGCTTCTACTACTCGATCTTCTCGCTGTGCGTACAATACCT
GGTGCCCATCCTGATCGTCTCGGTGGCATACTTCGGGATCTACAACAAGC
TGAAGAGCCGCATCACCGTGGTGGCTGTGCAGGCGTCCTCCGCTCAGCGG
AAGGTGGAGCGGGGGCGGCGGATGAAGCGCACCAACTGCCTACTGATCAG
CATCGCCATCATCTTTGGCGTTTCTTGGCTGCCGCTGAACTTTTTCAACC
TGTACGCGGACATGGAGCGCTCGCCGGTCACTCAGAGCATGCTAGTCCGC
TACGCCATCTGCCACATGATCGGCATGAGCTCCGCCTGCTCCAACCCGTT
GCTCTACGGCTGGCTCAACGACAACTTCCGCTGCAACGTCCAGGCGGCGG
CGCGCAAGCGTCGCAAGTTGGGCGCCGAACTCTCCAAAGGCGAACTCAAG
CTGCTGGGGCCAGGCGGCGCCCAGAGCGGTACCGCCGGCGGGGAAGGCGG
TCTGGCGGCCACCGACTTCATGACCGGCCACCACGAGGGCGGACTGCGCA
GCGCCATAACCGAGTCGGTGGCCCTCACGGACCACAACCCCGTGCCCTCG
GAGGTCACCAAGCTGATGCCGCGGTA
The following amino acid sequence (SEQ ID NO:6) is the amino acid se-
quence for the protein encoded by the DNA sequence of SEQ ID NO:5:
MNQTEPAQLADGEHLSGYASSSNSVRYLDDRHPLDYLDLGTVHALNTTAINTSDLNETGSRPLDPVLIDRF
LSNRAVDSPWYHMLISMYGVLIVFGALGNTLVVIAVIRKPIMRTARNLFILNLAISDLLLCLVTMPLTLME
ILSKYWPYGSCSILCKTIAMLQALCIFVSTISITAIAFDRYQVIVYPTRDSLQFVGAVTILAGIWALALLL
ASPLFVYKELINTDTPALLQQIGLQDTIPYCIEDWPSRNGRFYYSIFSLCVQYLVPILIVSVAYFGIYNKL
KSRITVVAVQASSAQRKVERGRRMKRTNCLLISIAIIFGVSWLPLNFFNLYADMERSPVTQSMLVRYAICH
MIGMSSACSNPLLYGWLNDNFRCNVQAAARKRRKLGAELSKGELKLLGPGGAQSGTAGGEGGLAATDFMTG
HHEGGLRSAITESVALTDHNPVPSEVTKLMPR
The following DNA sequence for DmGPCR4 (SEQ ID NO:7) was identified in
D. melanogaster:
ATGGAGAACACCACAATGCTGGCTAATATTAGCCTAAATGCAACCAGAAA
TGAGGAGAATATCACCTCATTCTTCACCGACGAAGAGTGGCTGGCCATCA
ATGGCACTTTGCCGTGGATAGTGGGATTCTTCTTCGGCGTCATCGCCATC
ACGGGATTCTTCGGCAACCTGCTGGTCATCCTGGTGGTGGTCTTCAACAA
CAACATGCGCTCCACCACCAACCTGATGATTGTCAATCTGGCTGCCGCTG
ATCTGATGTTCGTAATCCTCTGCATTCCCTTCACGGCCACCGATTACATG
GTGTACTACTGGCCATATGGAAGGTTCTGGTGCCCCAGTGTCCAGTACCT
GATTGTGGTGACCGCCTTCGCCTCCATCTACACGCTGGTGCTAATGTCCA
TCGATCGGTTCCTGGCGGTGGTTCATCCCATTCGCTCGCGGATGATGAGG
ACGGAGAACATTACCCTGATTGCCATCGTGACTCTGTGGATCGTGGTGCT
GGTCGTTTCGGTGCCAGTGGCCTTCACCCACGACGTGGTGGTGGACTACG
ATGCAAAGAAGAACATCACCTACGGCATGTGCACCTTCACGACGAACGAC
TTCCTTGGTCCGCGCACCTACCAGGTCACCTTCTTCATCAGCTCCTACCT
GCTGCCCCTGATGATCATCAGCGGTCTCTACATGCGCATGATCATGCGGC
TCTGGCGCCAGGGAACCGGCGTCCGCATGTCCAAGGAGTCGCAGCGCGGT
CGCAAGCGGGTCACCCGACTCGTCGTCGTGGTGGTCATCGCCTTCGCCTC
GCTCTGGCTGCCTGTCCAGCTCATCCTGCTGCTCAAGTCACTGGATGTCA
TCGAGACGAACACCCTCACCAAGCTAGTCATCCAGGTCACCGCCCACACT
CTGGCCTACAGCAGCTCGTGTATCAATCCGCTGCTCTACGCCTTCCTCTC
CGAGAATTTCCGGAAGGCCTTCTATAAGGCCGTTAACTGCTCCTCTCGAT
ACCAGAACTACACATCTGATTTGCCGCCGCCGCGCAAGACGTCCTGTGCC
AGGACCTCCACCACTGGACTCTA
The following amino acid sequence (SEQ ID NO:8) is the amino acid se-
quence for the protein encoded by the DNA sequence of SEQ ID NO:7:
MENTTMLANISLNATRNEENITSFFTDEEWLAINGTLPWIVGFFFGVIAITGFFGNLLVILVVVFNNNMRS
TTNLMIVNLAAADLMFVILCIPFTATDYMVYYWPYGRFWCRSVQYLIVVTAFASIYTLVLMSIDRFLAVVH
PIRSRMMRTENITLIAIVTLWIVVLVVSVPVAFTHDVVVDYDAKKNITYGMCTFTTNDFLGPRTYQVTFFI
SSYLLPLMIISGLYMRMIMRLWRQGTGVRMSKESQRGRKRVTRLVVVVVIAFASLWLPVQLILLLKSLDVI
ETNTLTKLVIQVTAQTLAYSSSCINPLLYAFLSENFRKAFYKAVNCSSRYQNYTSDLPPPRKTSCARTSTT
GL
The following DNA sequence for DmGPCR5a (SEQ ID NO:9) was identified in
D. melanogaster:
ATGGAGAATCGCAGTGACTTCGAGGCGGATGACTACGGCGACATCAGTTG
GAGCAATTGGAGCAACTGGAGCACCCCCGCCGGCGTCCTTTTCTCGGCCA
TGAGCAGCGTGCTCTCGGCCAGCAACCATACGCCCCTGCCGGACTTTGGC
CAGGAGCTCGCCCTATCCACCAGCTCCTTCAATCACAGCCAGACCCTATC
CACCGACCAGCCCGCCGTCGGGGACGTGGAAGACGCGGCCGAGGATGCGG
CGGCGTCCATGGAGACGGGCTCGTTTGCATTTGTGGTCCCGTGGTGGCGT
CAGGTGCTCTGGAGCATCCTCTTCGGCGGCATGGTCATTGTGGCGACGGG
CGGTAACCTGATTGTTGTCTGGATCGTGATGACGACCAAGCGGATGCGGA
CGGTAACCAACTATTTCATAGTGAATCTCTCCATCGCGGACGCCATGGTG
TCCAGCCTAAACGTCACCTTCAACTACTACTATATGCTGGATAGCGACTG
GCCCTTCGGCGAGTTCTACTGCAAGTTGTCCCAGTTCATCGCGATGCTAA
GCATCTGCGCCTCAGTGTTCACCCTAATGGCCATCTCCATCGACAGATAC
GTGGCCATCATCCGGCCACTGCAGCCGCGGATGAGCAAGCGGTGCAACCT
GGCCATCGCGGCGGTCATCTGGCTGGCCTCCACGCTCATCTCCTGCCCCA
TGATGATCATCTACCGCACGGAGGAGGTGCCGGTCCGCGGGCTCAGCAAC
CGCACGGTCTGCTACCCGGAGTGGCCCGATGGGCCCACCAATCACTCCAC
GATGGAGTCCCTCTACAACATCCTCATCATCATYCTAACCTACTTCCTGC
CCATCGTCTCCATGACGGTCACCTACTCGCGCGTGGGCATCGAGCTCTGG
GGATCCAAGACCATCGGCGAGTGCACGCCCCGCCAGGTGGARAAYGTGCG
GAGTAAGCGAAGGGTGGTGAAGATGATGATTGTGGTCGTCCTGATATTCG
CCATCTGCTGGCTGCCGTTCCACAGCTACTTCATAATCACATCCTGCTAC
CCGGCCATCACGGAGGCGCCCTTCATCCAGGAACTCTACCTGGCCATCTA
CTGGCTGGCCATGAGCAACTCCATGTACAATCCCATTATATACTGCTGGA
TGAATTCGCGCTTTCGCTATGGTTTCAAGATGGTCTTCCGCTGGTGCCTG
TTTGTGCGCGTGGGCACTGAACCCTTTAGTCGGCGGGAGAACCTGACATC
CCGGTACTCCTGCTCCGGTTCCCCGGATCACAATCGCATCAAGCGCAATG
ATACCCAGAAATCGATACTTTATACCTGTCCCAGCTCACCCAAGTCGCAT
CGAATTTCGCACAGCGGAACAGGTCGCAGTGCGACGCTGCGGAACAGTCT
GCCGGCGGAGTCACTGTCGTCCGGCGGATCTGGTGGTGGAGGGCACAGGA
AACGGTTGTCCTACCAGCAGGAAATGCAGCAGCGTTGGTCAGGACCCAAT
AGTGCCACCGCAGTGACCAATTCCAGCAGTACGGCCAACACCACCCAACT
GCTCTCCTG
The following amino acid sequence (SEQ ID NO:10) is the amino acid se-
quence for the protein encoded by the DNA sequence of SEQ ID NO:9:
MENRSDFEADDYGDISWSNWSNWSTPAGVLFSAMSSVLSASNHTPLPDFGQELALSTSSFNHSQTLSTDQP
AVGDVEDAAEDAAASMETGSFAFVVPWWRQVLWSILFGGMVIVATGGNLIVVWIVMTTKRMRTVTNYFIVN
LSIADAMVSSLNVTFNYYYMLDSDWPFGEFYCKLSQFIAMLSICASVFTLMAISIDRYVAIIRPLQPRMSK
RCNLAIAAVIWLASTLISCPMMIIYRTEEVPVRGLSNRTVCYPEWPDGPTNHSTMESLYNILIIILTYFLP
IVSMTVTYSRVGIELWGSKTIGECTPRQVENVRSKRRVVKNMIVVVLIFAICWLPFHSYFIITSCYPAITE
APFIQELYLAIYWLAMSNSMYNPIIYCWMNSRFRYGFKMVFRWCLFVRVGTEPFSRRENLTSRYSCSGSPD
HNRIKRNDTQKSILYTCPSSPKSHRISHSGTGRSATLRNSLPAESLSSGGSGGGGHRKRLSYQQEMQQRWS
GPNSATAVTNSSSTANTTQLLS
The following DNA sequence for DmGPCR5b (SEQ ID NO:11) was identified in
D. melanogaster:
ATGGAGAATCGCAGTGACTTCGAGGCGGATGACTACGGCGACATCAGTTG
GAGCAATTGGAGCAATTGGAGCAACTGGAGCACCCCCGCCGGCGTCCTTT
TCTCGGCCATGAGCAGCGTGCTCTCGGCCAGCAACCATACGCCTCTGCCG
GACTTTGGCCAGGAGCTCGCCCTATCCACCAGCTCCTTCAATCACAGCCA
GACCCTATCCACCGACCTGCCCGCCGTCGGGGACGTGGAAGACGCGGCCG
AGGATGCGGCGGCGTCCATGGAGACGGGCTCGTTTGCATTTGTGGTCCCG
TGGTGGCGTCAGGTGCTCTGGAGCATCCTCTTCGGCGGCATGGTCATTGT
GGCGACGGGCGGTAACCTGATTGTTGTCTGGATCGTGATGACGACCAAGC
GGATGCGGACGGTAACCAACTATTTCATACTAAATCTCTCCATCGCGGAC
GCCATGGTGTCCAGCCTGAACGTCACCTTCAACTACTACTACATGCTGGA
TAGCGACTGGCCCTTCGGCGAGTTCTACTGCAAGTTGTCCCAGTTCATCG
CGATGCTAAGCATCTGCGCCTCAGTGTTCACCCTAATGGCCATCTCCATC
GACAGATACGTGGCCATCATCCGGCCACTGCAGCCGCGGATGAGCAAGCG
GTGCAACCTGGCCATCGCGGCGGTCATCTGGCTGGCCTCCACGCTCATCT
CCTGCCCCATGATGATCATCTACCGCACGGAGGAGGTGCCGGTCCGCGGG
CTCAGCAACCGCACGGTCTGCTACCCGGAGTGGCCCGATGGGCCCACCAA
TCACTCCACGATGGAGTCCCTCTACAACATCCTCATCATCATTCTAACCT
ACTTCCTGCCCATCGTCTCCATGACGGTCACCTACTCGCGCGTGGGCATC
GAGCTCTGGGGATCCAAGACCATCGGCGAGTGCACGCCCCGCCAGGTGGA
GAATGTGCGGAGTAAGCGAAGGGTGGTGAAGATGATGATTGTGGTCGTCC
TGATATTCGCCATCTGCTGGCTGCCGTTCCACAGCTACTTCATAATCACA
TCCTGCTACCCGGCCATCACGGAGGCGCCCTTCATCCAGGAACTTTACCT
GGCCATCTACTGGCTGGCCATGAGCAACTCCATGTACAATCCCATTATAT
ACTGCTGGATGAATTCGCGCTTTCGCTATGGTTTCAAGATGGTCTTCCGC
TGGTGCCTGTTTGTGCGCGTGGGCACTGAACCCTTTAGTCGGCGGGAGAA
CCTGACATCCCGGTACTCCTGCTCCGGTTCCCCGGATCACAATCGCATCA
AGCGCAATGATACCCAGAAATCGATACTTTATACCTGTCCCAGCTCACCC
AAGTCGCATCGAATTTCGCACAGCGGAACAGGTCGCAGTGCGACGCTGAG
GAACAGTCTGCCGGCGGAGTCATTGTCGTCCGGTGGATCTGGAGCTGGAG
GACACAGGAAACGGTTGTCCTACCAGCAGGAAATGCAGCAGCGGTGGTCA
GGACCCAATAGTGCCACCGCAGTGACCAATTCCAGCAGTACGGCCAACAC
CACCCAACTGCTCTCCTG
The following amino acid sequence (SEQ ID NO:12) is the amino acid se-
quence for the protein encoded by the DNA sequence of SEQ ID NO:11:
MENRSDFEADDYGDISWSNWSNWSNWSTPAGVLFSAMSSVLSASNHTPLPDFGQELALSTSSFNHSQTLST
DLPAVGDVEDAAEDAAASMETGSFAFVVPWWRQVLWSILFGGMVIVATGGNLIVVWIVMTTKRMRTVTNYF
IVNLSIADAMVSSLNVTFNYYYMLDSDWPFGEFYCKLSQFIAMLSICASVFTLMAISIDRYVAIIRPLQPR
MSKRCNLAIAAVIWLASTLISCPMMIIYRTEEVPVRGLSNRTVCYPEWPDGPTNHSTMESLYNILIIILTY
FLPIVSMTVTYSRVGIELWGSKTIGECTPRQVENVRSKRRVVKMMIVVVLIFAICWLPFHSYFIITSCYPA
ITEAPFIQELYLAIYWLAMSNSMYNPIIYCWMNSRFRYGFKMVFRWCLFVRVGTEPFSRRENLTSRYSCSG
SPDHNRIKRNDTQKSILYTCPSSPKSHRISHSGTGRSATLRNSLPAESLSSGGSGGGGHRKRLSYQQEMQQ
RWSGPNSATAVTNSSSTANTTQLLS
The following DNA sequence for DmGPCR6aL (SEQ ID NO:13) was identified
in D. melanogaster:
ATGGAGCACCACAATAGCCATCTGTTGCCTGGTGGCAGCGAGAAGATGTA
CTACATAGCTCACCAGCAGCCGATGCTGCGGAACGAGGATGATAACTACC
AGGAGGGGTACTTCATCAGGCCGGACCCTGCATCCTTACTTTACAATACC
ACCGCACTGCCAGCGGACGATGAAGGGTCCAACTATGGATATGGCTCCAC
CACAACGCTCAGTGGCCTCCAGTTCGAGACCTATAATATCACTGTGATGA
TGAACTTTAGCTGTGACGACTATGACCTTCTATCGGAGGACATGTGGTCT
AGTGCCTACTTTAAGATCATCGTCTACATGCTCTACATTCCCATCTTTAT
CTTCGCCCTGATCGGCAACGGAACGGTCTGCTATATCGTCTATTCCACAC
CTCGCATGCGCACGGTCACCAATTACTTTATAGCCAGCTTGGCCATCGGC
GACATCCTGATGTCCTTCTTCTGCGTTCCGTCGTCCTTCATCTCGCTGTT
CATCCTGAACTACTGGCCTTTTGGCCTGGCCCTCTGTCACTTTCTGAACT
ACTCGCAGGCGGTCTCAGTTCTGGTCAGCGCCTATACTTTGGTGGCAATT
AGCATTGACCGCTACATAGCCATTATGTGGCCATTAAAGCCACGCATCAC
AAAACGCTATGCCACCTTCATCATCGCCGGCGTTTGGTTTATTGCACTTG
CCACCGCACTTCCCATACCCATCGTCTCTGGACTCGACATCCCAATGTCG
CCGTGGCACACGAAATGCGAGAAATACATTTGCCGCGAAATGTGGCCGTC
GCGGACGCAGGAGTACTACTACACCCTGTCCCTCTTCGCGCTGCAGTTCG
TCGTGCCGCTGGGCGTGCTCATCTTCACCTACGCCCGGATCACCATTCGC
GTCTGGGCGAAACGACCGCCAGGCGAGGCGGAAACCAACCGCGACCAGCG
GATGGCACGCTCCAAACGGAAGATGGTCAAAATGATGCTGACGGTTGTGA
TTGTGTTCACCTGCTGTTGGCTGCCCTTCAATATTTTGCAGCTTTTACTG
AACGACGAGGAGTTCGCCCACTGGGATCCTCTGCCGTATGTATGGTTCGC
GTTTCACTGGCTGGCCATGTCGCACTGCTGCTACAATCCGATCATCTACT
GCTACATGAACGCCCGTTTCAGGAGCGGATTCGTCCAGCTGATGCACCGT
ATGCCCGGCCTGCGTCGCTGGTGCTGCCTGCGGAGCGTCGGTGATCGCAT
GAACGCAACTTCCGGAACGGGTCCAGCACTTCCTCTCAATCGAATGAACA
CATCCACCACCTACATCAGCGCTCGTCGAAAGCCACGAGCGACATCTTTG
CGAGCGAACCCATTATCATGCGGCGAGACGTCACCACTGCGGTA
The following amino acid sequence (SEQ ID NO:14) is the amino acid se-
quence for the protein encoded by the DNA sequence of SEQ ID NO:13:
MEHHNSHLLPGGSEKMYYIAHQQPMLRNEDDNYQEGYFIRPDPASLLYNTTALPADDEGSNYGYGSTTTLS
GLQFETYNITVMMNFSCDDYDLLSEDMWSSAYFKIIVYMLYIPIFIFALIGNGTVCYIVYSTPRMRTVTNY
FIASLAIGDILMSFFCVPSSFISLFILNYWPFGLALCHFVNYSQAVSVLVSAYTLVAISIDRYIAIMWPLK
PRITKRYATFIIAGVWFIALATALPIPIVSGLDIPMSPWHTKCEKYICREMWPSRTQEYYYTLSLFALQFV
VPLGVLIFTYARITIRVWAKRPPGEAETNRDQRMARSKRKMVKMMLTVVIVFTCCWLPFNILQLLLNDEEF
AHWDPLPYVWFAFHWLAMSHCCYNPIIYCYMNARFRSGFVQLMHRMPGLRRWCCLRSVGDRMNATSGTGPA
LPLNRMNTSTTYISARRKPRATSLRANPLSCGETSPLR
The following DNA sequence for DmGPCR6bL (SEQ ID NO:15) was identified
in D. melanogaster:
ATGGAGCACCACAATAGCCATCTGTTGCCTGGTGGCAGCGAGAAGATGTA
CTACATAGCTCACCAGCAGCCGATGCTGCGGAACGAGGATGATAACTACC
AGGAGGGGTACTTCATCAGGCCGGACCCTGCATCCTTACTTTACAATACC
ACCGCACTGCCAGCGGACGATGAAGGGTCCAACTATGGATATGGCTCCAC
CACAACGCTCAGTGGCCTCCAGTTCGAGACCTATAATATCACTGTGATGA
TGAACTTTAGCTGTGACGACTATGACCTTCTATCGGAGGACATGTGGTCT
AGTGCCTACTTTAAGATCATCGTCTACATGCTCTACATTCCCATCTTTAT
CTTCGCCCTGATCGGCAACGGAACGGTCTGCTATATCGTCTATTCCACAC
CTCGCATGCGCACGGTCACCAATTACTTTATAGCCAGCTTGGCCATCGGC
GACATCCTGATGTCCTTCTTCTGCGTTCCGTCGTCCTTCATCTCGCTGTT
CATCCTGAACTACTGGCCTTTTGGCCTGGCCCTCTGTCACTTTGTGAACT
ACTCGCAGGCGGTCTCAGTTCTGGTCAGCGCCTATACTTTGGTCGCAATT
AGCATTGACCGCTACATAGCCATTATGTGGCCATTAAAGCCACGCATCAC
AAAACGCTATGCCACCTTCATCATCGCCGGCGTTTGGTTTATTGCACTTG
CCACCGCACTTCCCATACCCATCGTCTCTGGACTCGACATCCCAATGTCG
CCGTGGCACACGAAATGCGAGAAATACATTTGCCGCGAAATGTGGCCGTC
GCGGACGCAGGAGTACTACTACACCCTGTCCCTCTTCGCGCTGCAGTTCG
TCGTGCCGCTGGGCGTGCTCATCTTCACCTACGCCCGGATCACCATTCGC
GTCTGGGCGAAACGACCGCCAGGCGAGGCGGAAACCAACCGCGACCAGCG
GATGGCACGCTCCAAACGGAAGATGGTCAAAATGATGCTGACGGTTGTGA
TTGTGTTCACCTGCTGTTGGCTGCCCTTCAATATTTTGCAGCTTTTACTG
AACGACGAGGAGTTCGCCCACTGGGATCCTCTGCCGTATGTGTGGTTCGC
GTTTCACTGGCTGGCCATGTCGCACTGCTGCTACAATCCGATCATCTACT
GCTACATGAACGCCCGTTTCAGGAGCGGATTCGTCCAGCTGATGCACCGT
ATGCCCGGCCTGCGTCGCTGGTGCTGCCTGCGGAGCGTCGGTGATCGCAT
GAACGCAACTTCCGGTGAGATGACTACGAAGTACCATCGCCATGTCGGCG
ATGCCCTATTCCGGAAACCCAAAATATGCATTAGGAACGGGTCCAGCACT
TCCTCTCAATCGAATGAACACATCCACCACCTACATCAGCGCTCGTCGAA
AGCCACGAGCGACATCTTTGCGAGCGAACCCATTATCATGCGGCGAGACG
TCACCACTGCGGTAGCTGTCATATCAAAAAATAAAACTGATTCACCGGTG
CGCCGATCGGGAAGCTCAGGTGGAACAGAAGCAAACATAAGAAGCACCGA
GTTTTG
The following amino acid sequence (SEQ ID NO:16) is the amino acid se-
quence for the protein encoded by the DNA sequence of SEQ ID NO:15:
MEHHNSHLLPGGSEKMYYIAHQQPMLRNEDDNYQEGYFIRPDPASLLYNTTALPADDEGSNYGYGSTTTLS
GLQFETYNITVMMNFSCDDYDLLSEDMWSSAYFKIIVYMLYIPIFIFALIGNGTVCYIVYSTPRMRTVTNY
FIASLAIGDILMSFFCVPSSFISLFILNYWPFGLALCHFVNYSQAVSVLVSAYTLVAISIDRYIAIMWPLK
PRITKRYATFIIAGVWFIALATALPIPIVSGLDIPMSPWHTKCEKYICREMWPSRTQEYYYTLSLFALQFV
VPLGVLIFTYARITIRVWAKRPPGEAETNRDQRMARSKRKMVKMMLTVVIVFTCCWLPFNILQLLLNDEEF
AHWDPLPYVWFAFHWLAMSHCCYNPIIYCYMNARFRSGFVQLMHRMPGLRRWCCLRSVGDRMNATSGEMTT
KYHRHVGDALFRKPKICIRNGSSTSSQSNEHIHHLHQRSSKATSDIFASEPIIMRRDVTTAVAVISKNKTD
SPVRRSGSSGGTEANIRSTEF
The following DNA sequence for DmGPCR7 (SEQ ID NO:17) was identified in
D. melanogaster:
ATGGCAATGGACTTAATCGAGCAGGAGTCCCGCCTGGAATTCCTGCCCGG
AGCCGAGGAGGAAGCAGAATTTGAGCGTCTATACGCGGCTCCCGCTGAGA
TTGTGGCCCTGTTGTCCATTTTCTATGGGGGAATCAGTATCGTGGCCGTC
ATTGGCAACACTTTGGTCATCTGGGTGGTGGCCACGACCAGGCAAATGCG
GACCGTGACAAATATGTATATCGCTAATTTGGCTTTTGCCGATGTGATTA
TTGGCCTCTTCTGCATACCATTTCAGTTCCAGGCTGCCCTGCTGCAGAGT
TGGAACCTGCCGTGGTTCATGTGCAGCTTCTGCCCCTTCGTCCAGGCCCT
GAGTGTAAATGTCTCGGTATTCACGCTGACCGCCATTGCAATCGATCGGC
ATAGGGCCATCATTAATCCACTTAGGGCACGTCCCACCAAGTTCGTATCG
AAGTTCATAATTGGTGGAATTTGGATGCTGGCCCTGCTATTTGCGGTGCC
CTTTGCCATTGCCTTTCGTGTGGAGGAGTTGACCGAAAGATTTCGCGAGA
ACAATGAGACCTACAATGTGACGCGGCCATTCTGCATGAACAAGAACCTA
TCCGATGATCAATTGCAATCCTTTCGCTACACCCTGGTTTTTGTGCAGTA
TCTGGTTCCATTCTGTGTCATCAGCTTTGTCTACATCCAGATGGCGGTAC
GATTGTGGGGCACACGTGCTCCTGGTAACGCACAGGATTCACGGGACATA
ACGCTGTTGAAAAACAAGAAGAAGGTCATCAAAATGCTGATTATCGTGGT
CATTATCTTTGGACTCTGCTGGCTGCCACTGCAGCTCTATAATATTCTGT
ATGTCACGATACCGGAAATCAACGACTACCACTTCATTAGCATCGTCTGG
TTTTGCTGCGATTGGCTGGCCATGAGCAATAGCTGCTACAATCCCTTTAT
TTATGGCATCTACAATGAAAAATTTAAGCGGGAATTCAACAAGCGATTTG
CGGCCTGTTTCTGCAAGTTCAAGACGAGCATGGACGCCCACGAAAGGACC
TTTTCGATGCACACCCGCGCCAGCTCCATAAGGTCAACCTACGCCAACTC
CTCGATGCGAATCCGGAGTAATCTCTTTGGTCCGGCGCGTGGTGGTGTCA
ACAATGGGAAGCCGGGCTTGCATATGCCGCGGGTGCATGGATCCGGTGCT
AACAGCGGCATTTACAACGGAAGTAGTGGGCAGAACAACAATGTCAATGG
CCAACATCATCAGCATCAAAGCGTGGTTACCTTTGCGGCCACTCCGGGTG
TTTCGGCACCAGGTGTTGGCGTTGCAATGCCGCCGTGGCGGCGAAACAAC
TTCAAACCTCTGCATCCGAACGTAATCGAATGCGAGGACGACGTGGCACT
CATGGAGCTGCCATCAACCACGCCCCCCAGCGAGGAGTTGGCATCCGGGG
CCGGAGTCCAGTTGGCCCTGCTAAGCAGGGAGAGCTCCAGCTGCATTTGC
GAACAGGAATTTGGCAGCCAAACCGAATGCGATGGCACCTGCATACTCAG
CGAGGTGTCGCGAGTCCACCTGCCCGGCTCGCAGGCGAAGGACAAGGATG
CGGGCAAGTCCTTGTGGCAACCACTTTA
The following amino acid sequence (SEQ ID NO:18) is the amino acid se-
quence for the protein encoded by the DNA sequence of SEQ ID NO:17:
MAMDLIEQESRLEFLPGAEEEAEFERLYAAPAEIVALLSIFYGGISIVAVIGNTLVIWVVATTRQMRTVTN
MYIANLAFADVIIGLFCIPFQFQAALLQSWNLPWFMCSFCPFVQALSVNVSVFTLTAIAIDRHRAIINPLR
ARPTKFVSKFIIGGIWMLALLFAVPFAIAFRVEELTERFRENNETYNVTRPFCMNKNLSDDQLQSFRYTLV
FVQYLVPFCVISFVYIQMAVRLWGTRAPGNAQDSRDITLLKNKKKVIKMLIIVVIIFGLCWLPLQLYNILY
VTIPEINDYHFISIVWFCCDWLAMSNSCYNPFIYGIYNEKFKREFNKRFAACFCKFKTSMDAHERTFSMHT
RASSIRSTYANSSMRIRSNLFGPARGGVNNGKPGLHMPRVHGSGANSGIYNGSSGQNNNVNGQHHQHQSVV
TFAATPGVSAPGVGVAMPPWRRNNFKPLHPNVIECEDDVALMELPSTTPPSEELASGAGVQLALLSRESSS
CICEQEFGSQTECDGTCILSEVSRVHLPGSQAKDKDAGKSLWQPL
The following DNA sequence for DmGPCR8 (SEQ ID NO:19) was identified in
D. melanogaster:
ATGTTTACGTGGCTGATGATGGATGTCCTCCAGTTTGTGAAAGGGGAAAT
GACAGCCGATTCAGAGGCAAATGCCACAAATTGGTATAACACGAACGAGA
GCTTATATACCACGGAACTGAACCATAGATGGATTAGTGGTAGTTCCACA
ATTCAGCCAGAGGAGTCCCTTTATGGCACTGATTTGCCCACCTATCAACA
TTGCATAGCCACGCGGAATTCCTTTGCTGACTTGTTCACTGTGGTGCTCT
ACGGATTTGTGTGCATTATCGGATTATTTGGCAACACCCTGGTGATCTAC
GTGGTGTTGCGCTTTTCCAAAATGCAAACGGTCACGAATATATATATCCT
GAATCTCGCGGTGGCAGACGAGTGCTTCCTGATTGGAATACCCTTTCTGC
TGTACACAATGCGAATTTGCAGCTGGCGATTCGGGGAGTTTATGTGCAAA
GCCTACATGGTGAGCACATCCATCACCTCCTTCACCTCGTCGATTTTTCT
GCTCATCATGTCCGCGGATCGATATATAGCGGTATGCCACCCGATTTCCT
CGCCACGATATCGAACTCTGCATATTGCCAAAGTGGTCTCAGCGATTGCC
TGGTCAACTTCAGCGGTCCTCATGCTGCCCGTGATCCTTTATGCCAGCAC
TGTGGAGCAGGAGGATGGCATCAATTACTCGTGCAACATAATGTGGCCAG
ATGCGTACAAGAAGCATTCGGGCACCACCTTCATACTGTACACATTTTTC
CTAGGATTCGCCACACCGCTGTGCTTTATCCTGAGTTTCTACTACTTGGT
TATAAGGAAACTGCGATCGGTGGGTCCCAAACCAGGAACGAAGTCCAAGG
AGAAGAGGCGGGCTCACAGGAAGGTCACTCGACTGGTACTGACGGTGATA
AGTGTATACATTCTATGTTGGCTCCCTCACTGGATTTCTCAGGTGGCCCT
GATTCACTCGAATCCCGCGCAAAGGGACCTCTCCCGACTGGAAATACTCA
TTTTCCTACTTCTGGGGGCACTGGTTTACTCGAATTCGGCGGTGAATCCC
ATACTTTATGCCTTCCTAAGTGAGAACTTCCGGAAGAGCTTCTTCAAGGC
CTTTACCTGTATGAATAAGCAGGATATCAACGCTCAACTCCAGCTGGAGC
CCAGTGTTTTCACCAAACAGGGCAGTAAAAAGAGGGGTGGCTCCAAGCGC
CTGTTGACCAGCAATCCGCAGATTCCTCCACTGCTGCCACTGAATGCGGG
TAACAACAATTCATCGACCACCACATCCTCGACCACGACAGCGGAAAAGA
CCGGAACCACGGGGACACAGAAATCATGCAATTCCAATGGCAAAGTGACA
GCTCCGCCGGAGAATTTGATTATATGTTTGAGCGAGCAGCAGGAGGCATT
TTGCACCACCGCGAGAAGAGGATCGGGCGCAGTGCAGCAGACAGATTTGT
A
The following amino acid sequence (SEQ ID NO:20) is the amino acid se-
quence for the protein encoded by the DNA sequence of SEQ ID NO:19:
MFTWLMMDVLQFVKGEMTADSEANATNWYNTNESLYTTELNHRWISGSSTIQPEESLYGTDLPTYQHCIAT
RNSFADLFTVVLYGFVCIIGLFGNTLVIYVVLRFSKMQTVTNIYILNLAVADECFLIGIPFLLYTMRICSW
RFGEFMCKAYMVSTSITSFTSSIFLLIMSADRYIAVCHPISSPRYRTLHIAKVVSAIAWSTSAVLMLPVIL
YASTVEQEDGINYSCNIMWPDAYKKHSGTTFILYTFFLGFATPLCFILSFYYLVIRKLRSVGPKPGTKSKE
KRRAHRKVTRLVLTVISVYILCWLPHWISQVALIHSNPAQRDLSRLEILIFLLLGALVYSNSAVNPILYAF
LSEHFRKSFFKAFTCMNKQDINAQLQLEPSVFTKQGSKKRGGSKRLLTSNPQIPPLLPLNAGNNNSSTTTS
STTTAEKTGTTGTQKSCNSNGKVTAPPENLIICLSEQQEAFCTTARRGSGAVQQTDL
The following DNA sequence for DmGPCR9 (SEQ ID NO:21) was identified in
D. melanogaster:
ATGTTCAACTACGAGGAGGGGGATGCCGACCAGGCGGCCATGGCTGCAGC
GGCTGCCTATAGGGCACTGCTCGACTACTATGCCAATGCGCCAAGTGCGG
CGGGTCACATAGTGTCGCTCAACGTGGCACCCTACAATGGAACTGGAAAC
GGAGGCACTGTCTCCTTGGCGGGCAATGCGACAAGCAGCTATGGCGATGA
TGATAGGGATGGCTATATGGACACCGAGCCCAGTGACCTGGTCACCGAAC
TGGCCTTCTCCCTGGGCACCAGTTCAAGTCCAAGTCCCAGTTCCACACCC
GCTTCCAGCTCCAGTACTTCCACTGGCATGCCCGTCTGGCTGATACCCAG
CTATAGCATGATTCTGCTGTTCGCCGTGCTGGGCAACCTGCTGGTCATCT
CGACGCTGGTGCAGAATCGCCGGATGCGTACCATAACCAACGTGTTCCTG
CTCAACCTGGCCATATCGGACATGCTGCTGGGCGTGCTCTGCATGCCCGT
CACCCTGGTGGGCACCCTGCTGCGAAACTTCATCTTTGGCGAGTTCCTCT
GCAAGCTCTTTCAGTTCTCGCAAGCCGCCTCCGTGGCCGTTTCGTCCTGG
ACCTTGGTGGCCATATCCTGTGAGCGCTACTACGCGATATGCCATCCACT
GCGCTCGCGATCCTGGCAGACAATCAGTCACGCCTACAAGATCATCGGCT
TCATCTGGCTGGGCGGCATCCTCTGCATGACGCCCATAGCGGTCTTTAGT
CAATTGATACCCACCAGTCGACCGGGCTACTGCAAGTGCCGTGAGTTTTG
GCCCGACCAGGGATACGAGCTCTTCTACAACATCCTGCTGGACTTCCTGC
TGCTCGTCCTGCCGCTTCTCGTCCTCTGCGTGGCCTACATCCTCATCACG
CGTACCCTGTACGTAGGCATGGCCAAGGACAGCGGACGCATCCTGCAGCA
ATCGCTGCCTGTTTCCGCTACAACGGCCGGCGGAAGCGCACCGAATCCGG
GCACCAGCAGCAGTAGTAACTGCATCCTGGTCCTGACCGCCACCGCAGTC
TATAATGAAAATAGTAACAATAATAATGGAAATTCAGAGGGATCCGCAGG
CGGAGGATCAACCAATATGGCAACGACCACCTTGACAACGAGACCAACGG
CTCCAACTGTGATCACCACCACCACGACGACCACGGTGACGCTGGCCAAG
ACCTCCTCGCCCAGCATTCGCGTCCACGATGCGGCACTTCGCAGGTCCAA
CGAGGCCAAGACCCTGGAGAGCAAGAAGCGTGTGGTCAAGATGCTGTTCG
TCCTGGTGCTGGAGTTTTTCATCTGCTGGACTCCGCTGTACGTGATCAAC
ACGATGGTCATGCTGATCGGACCGGTGGTGTACGAGTATGTCGACTACAC
GGCCATCAGTTTCCTCCAGCTGCTGGCCTACTCATCCAGCTGCTGCAATC
CGATCACCTACTGCTTCATGAACGCCAGCTTCCGGCGCGCCTTTGTCGAC
ACCTTCAAGGGTCTGCCCTGGCGTCGTGGAGCAGGTGCCAGCGGAGGCGT
CGGTGGTGCTGCTGGTGGAGGACTCTCCGCCAGCCAGGCGGGCGCAGGCC
CGGGCGCCTATGCGAGTGCCAACACCAACATTAGTCTCAATCCCGGCCTA
GCCATGGGTATGGGCACCTGGCGGAGTCGCTCACGCCACGAGTTTCTCAA
TCCGGTGGTGACCACCAATAGTGCCGCCGCCGCCGTCAACAGTCCTCAGC
TCTA
The following amino acid sequence (SEQ ID NO:22) is the amino acid se-
quence for the protein encoded by the DNA sequence of SEQ ID NO:21:
MFNYEEGDADQAAMAAAAAYRALLDYYANAPSAAGHIVSLNVAPYNGTGNGGTVSLAGNATSSYGDDDRDG
YMDTEPSDLVTELAFSLGTSSSPSPSSTPASSSSTSTGMPVWLIPSYSMILLFAVLGNLLVISTLVQNRRM
RTITNVFLLNLAISDMLLGVLCMPVTLVGTLLRNFIFGEFLCKLFQFSQAASVAVSSWTLVAISCERYYAI
CHPLRSRSWQTISHAYKIIGFIWLGGILCMTPIAVFSQLIPTSRPGYCKCREFWPDQGYELFYNILLDFLL
LVLPLLVLCVAYILITRTLYVGMAKDSGRILQQSLPVSATTAGGSAPNPGTSSSSNCILVLTATAVYNENS
NNNNGNSEGSAGGGSTNMATTTLTTRPTAPTVITTTTTTTVTLAKTSSPSIRVHDAALRRSNEAKTLESKK
RVVKMLFVLVLEFFICWTPLYVINTMVMLIGPVVYEYVDYTAISFLQLLAYSSSCCNPITYCFMNASFRRA
FVDTFKGLPWRRGAGASGGVGGAAGGGLSASQAGAGPGAYASANTNISLNPGLAMGMGTWRSRSRHEFLNA
VVTTNSAAAAVNSPQL
The following DNA sequence for DmGPCR10 (SEQ ID NO:23) was identified in
D. melanogaster:
ATGTACGCCTCCTTGATGGACGTTGGCCAGACGTTGGCAGCCAGGCTGGCGGATAGCGAC
GGCAACGGGGCCAATGACAGCGGACTCCTGGCAACCGGACAAGGTCTGGAGCAGGAGCAG
GAGGGTCTGGCACTGGATATGGGCCACAATGCCAGCGCCGACGGCGGAATAGTACCGTAT
GTGCCCGTGCTGGACCGCCCGGAGACGTACATTGTCACCGTGCTGTACACGCTCATCTTC
ATTGTGGGAGTTTTGGGCAACGGCACGCTGGTCATCATCTTCTTTCGCCACCGCTCCATG
CGCAACATACCCAACACATACATTCTTTCACTGGCCCTGGCTGATCTGTTGGTTATATTG
GTGTGTGTACCTGTGGCCACGATTGTCTACACGCAGGAAAGCTGGCCCTTTGAGCGGAAC
ATGTGCCGCATCAGCGAGTTCTTTAAGGACATATCCATCGGGGTGTCCGTGTTTACACTG
ACCGCCCTTTCCGGCGAGCGGTACTGCGCCATTGTAAATCCCCTACGCAAGCTTCAGACC
AAGCCGCTCACTGTCTTTACTGCGGTGATGATCTGGATCCTGGCCATCCTACTGGGCATG
CCTTCGGTTCTTTTCTCCGACATCAAGTCCTACCCTGTGTTCACAGCCACCGGTAACATG
ACCATTGAAGTGTGCTCCCCATTTCGCGACCCGGAGTATGCAAAGTTCATGGTGGCGGGC
AAGGCACTGGTGTACTACCTGTTGCCGCTGTCCATCATTGGGGCGCTATACATCATGATG
GCCAAGCGGCTCCATATGAGCGCCCGCAACATGCCCGGCGAACAGCAGAGCATGCAGAGC
CGCACCCAGGCTAGGGCCCGACTCCATGTGGCGCGCATGGTGGTAGCATTCGTGGTGGTG
TTCTTCATCTGCTTCTTCCCGTACCACGTGTTTGAGCTGTGGTACCACTTCTACCCAACG
GCTGAGGAGGACTTCGATGAGTTCTGGAACGTGCTGCGCATCCTTCCTAAACTCGTGCGT
CAACCCCGTGGCCTCTACTGCGTGTCCGGGGTGTTTCGGCAGCACTTTAATCGCTACCTC
TGCTGCATCTGCGTCAAGCGGCAGCCGCACCTGCGGCAGCACTCAACGGCCACTGGAATG
ATGGACAATACCAGTGTGATGTCCATGCGCCGCTCCACGTACGTGGGTGGAACCGCTGGC
AATCTGCGGGCCTCGCTGCACCGGAACAGCAATCACGGAGTTGGTGGAGCTGGAGGTGGA
GTAGGAGGAGGAGTAGGGTCAGGTCGTGTGGGCAGCTTTCATCGGCAGGACTCGATGCCC
CTGCAGCACGGAAATGCCCACGGAGGTGGTGCGGGCGGGGGATCCTCCGGACTTGGAGCC
GGCGGGCGGACGGCGGCAGTGAGCGAAAAGAGCTTTATAAATCGTTACGAAAGTGGCGTA
ATGCGCTACTAA
The following amino acid sequence (SEQ ID NO:24) is the amino acid se-
quence for the protein encoded by the DNA sequence of SEQ ID NO:23:
MYASLMDVGQTLAARLADSDGNGANDSGLLATGQGLEQEQEGLALDMGHNASADGGIVPYVPVLDRPETYI
VTVLYTLIFIVGVLGNGTLVIIFFRHRSMRNIPNTYILSLALADLLVILVCVPVATIVYTQESWPFERNMC
RISEFFKDISIGVSVFTLTALSGERYCAIVNPLRKLQTKPLTVFTAVMIWILAILLGMPSVLFSDIKSYPV
FTATGNMTIEVCSPFRDPEYAKFMVAGKALVYYLLPLSIIGALYIMMAKRLHMSARNMPGEQQSMQSRTQA
RARLHVARMVVAFVVVFFICFFPYHVFELWYHFYPTAEEDFDEFWNVLRILPKLVRQPRGLYCVSGVFRQH
FNRYLCCICVKRQPHLRQHSTATGMMDNTSVMSMRRSTYVGGTAGNLRASLHRNSNHGVGGAGGGVGGGVG
SGRVGSFHRQDSMPLQHGNAHGGGAGGGSSGLGAGGRTAAVSEKSFINRYESGVMRY

In accordance with the Budapest Treaty, clones of the present invention have been deposited at the Agricultural Research Culture Collection (NRRL) International Depository Authority, 1815 N. University Street, Peoria, Ill. 61604, U.S.A. Accession numbers and deposit dates are provided below in Table 5.

TABLE 5

| Clone | NRRL Accession No. | Date of Deposit |
|---|---|---|
| DmGPCR1 (SEQ ID NO:1) | NRRL B-30347 | 19 Oct. 2000 |
| DmGPCR2a (SEQ ID NO:3) | NRRL B-30348 | 19 Oct. 2000 |
| DmGPCR4 (SEQ ID NO:7) | NRRL B-30349 | 19 Oct. 2000 |
| DmGPCR5a (SEQ ID NO:9) | NRRL B-30350 | 19 Oct. 2000 |
| DmGPCR6aL (SEQ ID NO:13) | NRRL B-30351 | 19 Oct. 2000 |
| DmGPCR6bL (SEQ ID NO:15) | NRRL B-30352 | 19 Oct. 2000 |
| DmGPCR7 (SEQ ID NO:17) | NRRL B-30353 | Oct. 19, 2000 |
| DmGPCR8 (SEQ ID NO:19) | NRRL B-30354 | Oct. 19, 2000 |
| DmGPCR9 (SEQ ID NO:21) | NRRL B-30355 | Oct. 19, 2000 |

The invention is further illustrated by way of the following examples which are intended to elucidate the invention. These examples are not intended, nor are they to be construed, as limiting the scope of the invention. It will be clear that the invention may be practiced otherwise than as particularly described herein. Numerous modifications and variations of the present invention are possible in view of the teachings herein and, therefore, are within the scope of the invention.

It is intended that each of the patents, applications, and printed publications mentioned in this patent document be hereby incorporated by reference in their entirety.

### EXAMPLES

### Example 1

### Identification of DmGPCRs

A Celera genomic D. melanogaster database was converted to a database of predicted proteins and a mRNA database using a variety of gene finding software tools to predict the mRNAs that would be generated (the “PnuFlyPep” database). Procedures for analyzing genomic databases using gene-finding software tools are known to those skilled in the art.

The nucleotide sequences of several C. elegans FaRP GPCRs were used as query sequences against the mRNA database described above. This database was searched for regions of similarity using a variety of tools, including FASTA and Gapped BLAST (Altschul et al., Nuc. Acids Res., 1997, 25, 3389, which is incorporated herein by reference in its entirety).

Briefly, the BLAST algorithm, which stands for Basic Local Alignment Search Tool is suitable for determining sequence similarity (Altschul et al., J. Mol. Biol., 1990, 215, 403-410, which is incorporated herein by reference in its entirety). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence that either match or satisfy some positive-valued threshold score T when aligned with a word of the same length in a database sequence. T is referred to as the neighbourhood word score threshold (Altschul et al., supra). These initial neighbourhood word hits act as seeds for initiating searches to find HSPs containing them. The word hits are extended in both directions along each sequence for as far as the cumulative alignment score can be increased. Extension for the word hits in each direction are halted when: 1) the cumulative alignment score falls off by the quantity X from its maximum achieved value; 2) the cumulative score goes to zero or below, due to the accumulation of one or more negative-scoring residue alignments; or 3) the end of either sequence is reached. The Blast algorithm parameters W, T, and X determine the sensitivity and speed of the alignment. The Blast program uses as defaults a word length (W) of 11, the BLOSUM62 scoring matrix (see Henikoff et al., Proc. Natl. Acad. Sci. USA, 1992, 89, 10915-10919, which is incorporated herein by reference in its entirety) alignments (B) of 50, expectation (E) of 10, M=5, N=4, and a comparison of both strands.

The BLAST algorithm (Karlin et al., Proc. Natl. Acad. Sci. USA, 1993, 90, 5873-5787, which is incorporated herein by reference in its entirety) and Gapped BLAST perform a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a nucleic acid is considered similar to a DmGPCR gene or cDNA if the smallest sum probability in comparison of the test nucleic acid to a DmGPCR nucleic acid is less than about 1, preferably less than about 0.1, more preferably less than about 0.01, and most preferably less than about 0.001.

The mRNAs corresponding to the predicted proteins were retrieved from the database of predicted mRNAs used to prepare the PnuFlyPep database. These are identified as the following nucleotide sequences: SEQ ID NOs: 1, 3, 5, 7, 9, 11, 13, 15, 17, 19, 21, and 23, each having a statistically significant overlapping homology to the query sequence. The nucleotide sequences SEQ ID NOs: 3, 5, 9, 11, 13, and 15 (corresponding to DmGPCRs 2a, 2b, 5a, 5b, 6a, and 6b) were obtained from PCR cloning and sequencing of another identified sequence (not shown). Each of these sequences represents a splice variant of a DmGPCR gene.

### Example 2

### Cloning of DmGPCRs

cDNA Preparation

cDNA was prepared from either adult Drosophila melanogaster poly A⁺ RNA (Clontech Laboratories, Palo Alto, Calif.) or adult Drosophila melanogaster total RNA (below). To obtain total RNA, parent stocks of Drosophila melanogaster (Biological Supply Company, Burlington, N.C.) were anesthetized by chilling, and 5 to 6 adults were added to a culture vessel containing 10 ml H₂O, 10 ml Formula 4-24 Instant Drosophila Medium, and 6 to 10 grains of active dry yeast (Biological Supply Company). A polyurethane foam plug was placed at end of each vessel, and flies were incubated at room temperature (RT) for 4 to 6 weeks. At maturity, the vessels were chilled, and the anesthetized flies were poured into a 50 ml polypropylene tube held in liquid N₂. The frozen flies were stored at −70° C. until they were ground with a mortar and pestle in the presence of liquid N₂. The powdered tissue along with some liquid N₂ was decanted into 50 ml polypropylene tubes on dry ice. Following evaporation of the liquid N₂, the powdered tissue was stored at −70° C.

To prepare RNA, 300 mg of powdered tissue was placed into polypropylene tubes on dry ice, and 5 ml of 6 M guanidine hydrochloride in 0.1 M NaOAc, pH 5.2 was added. All solutions were either treated with DEPC, or prepared with DEPC-treated dH₂O, and all glassware was baked, or virgin plastic labware was used, to reduce problems with RNase contamination. Tubes were vortex-mixed then placed on ice. The powdered tissue was homogenized by successive passage through 20, 21, and 22 gauge needles. The tubes were centrifuged (1000×g for 10 min), then 2.5 to 3 ml of supernatant was layered on top of 8 ml 5.7 M cesium chloride in 0.1 M NaOAc contained in 14×95 mm Ultra-Clear centrifuge tubes (Beckman Instruments, Inc., Palo Alto, Calif.). The samples were centrifuged at 25000 rpm for 18 h at 18° C. in an L8-70 ultracentrifuge (Beckman Instruments, Inc.,). The supernatant was decanted, and the tube was inverted and allowed to drain. The RNA pellet was suspended in 200 μl of RNase-free dH₂O (Qiagen Inc., Valencia, Calif.), then rinsed twice with 100 μl RNase-free dH₂O (total, 400 μl). The RNA was precipitated by the addition of 44 μl of 3M NaOAc, pH 5.2, and 1 ml cold 100% ethanol. Following overnight storage at −70° C., the tube was centrifuged at 14000 rpm for 1 h (Eppendorf microfuge 5402), rinsed with 75% ethanol (prepared with DEPC-treated dH₂O), then the pellet was dissolved in RNase-free dH₂O. Absorbances at 260 or 280 nm determined in 10 mM Tris-HCl, pH 7.5 were used to estimate RNA concentration and purity.

First-strand cDNA was prepared according to the procedure supplied with the Superscript II enzyme (GIBCO BRL, Rockville, Md.). Either 500 ng (2 μl) of poly A⁺ RNA or 3 μg (4 μl) of total RNA was added to microfuge tubes containing RNase-free dH₂O and 250 ng (2.5 μl) random primers. The tubes (12 μl) were incubated at 70° C. for 10 min, chilled on ice, then 4 μl of 5×first strand buffer, 2 μl of 0.1 M DTT, and 1 μl of 10 mM dNTP mix were added. Following incubation at 25° C. for 10 min, then at 42° C. for 2 min, 1 μl (200 units) of Superscript II was added, and incubation continued at 42° C. for 50 min. The enzyme was inactivated by incubation at 70° C. for 15 min. To remove RNA complimentary to the cDNA, 2 μl (2 units) of RNase H (Boehringer Mannheim, Indianapolis, Ind.) was added, followed by incubation at 37° C. for 20 min. The cDNA was stored at −20° C.

PCR Reactions

Either a standard 50/100 μl PCR reaction or Hot Start PCR Reaction, using Ampliwax beads (Perkin Elmer Cetus, Norwalk, Conn.) was used to amplify the Drosophila melanogaster G protein-coupled receptors (DmGPCRs). Distilled H₂O was used to dissolve the primers (Genosys Biotechnologies, Inc., The Woodlands, Tex.): 5′- and 3′-primers at 10 μM concentrations, internal primers at 1 μM. Each PCR reaction contained 2 to 4 units of rTth XL DNA polymerase, 1.2 to 1.5 mM Mg(OAc)₂, 2001M each dNTP, and 200 or 400 nM each primer. For Hot Start PCR, 32 or 36 μl ‘lower’ cocktail (dH₂O, 3.3×XL-buffer, dNTP and Mg(OAc)₂ was added to 2 or 4 μl of each primer (total volume, 40 μl). An Ampliwax bead (Perkin Elmer Cetus) was added, tubes incubated at 75° C. for 5 min, cooled at room temperature (RT), then 60 μl ‘upper’ cocktail (dH₂O, 3.3×XL-buffer, rTth and template) was added. PCR amplifications were performed in a Perkin Elmer Series 9600 thermal cycler. The typical program for the thermal cycler included: 1 min at 94° C., followed by 30 cycles of amplification (0.5 min at 94° C., 0.5 min at 60° C., 2 min at 72° C.), followed by 6 min at 60° C. In order to create 3′ A-overhangs on the PCR product (‘tailing’), 1 μl Taq polymerase (Invitrogen, Carlsbad, Calif.) was added at the end of the PCR amplification, and tubes incubated at 72° C. for 10 min. The reaction mixtures were analyzed on 1% agarose gel prepared in TAE buffer (5). PCR products were typically purified using QIAquick spun columns (QIAGEN).

Ligation and Transformation

Ligation of all PCR products into PCR 3.1 vector (Invitrogen) and transformation of the ligated products into One Shot™ TOP10F′ competent cells (Invitrogen) were done according to the manufacturer's directions. Transformants to be screened for inserts were propagated in LB broth containing 50 μg ampicillin/ml. Colonies with inserts were identified either by a boiling-lysis plasmid mini-prep procedure (5) or by a ‘colony PCR’ procedure that directly amplified the plasmid DNA from the transformed bacteria (6).

DNA Sequencing

DNA for sequencing was prepared using Qiagen anion-exchange plasmid kits (QIAGEN-tip 20) to isolate the DNA from 5 ml LB cultures grown at 37° C. overnight as per the manufacturer's directions. Four primers (T7, M13 reverse, ‘sense’ and ‘antisense’) were typically used for sequencing each DNA (Table 6). Dye-terminator sequencing chemistry was used, either the BigDye™ Terminator reagents (Applied Biosystems, Foster City, Calif.) or DYEnamic™ ET terminator kit (Amersham Pharmacia Biotech, Inc., Piscataway, N.J.). Manufacturer's recommendations were followed for preparation of the sequencing reactions. Primers and unincorporated nucleotides were removed using Centri-Sep spun columns (Princeton Separations, Adelphia, N.J.). Sequencing reactions were analyzed on an Applied Biosystems 377 automated DNA sequencer. DNA sequences were assembled and analyzed using Sequencher (Gene Codes, Ann Arbor, Mich.), the GCG group of sequence analysis programs (Wisconsin Package Version 10.1, Genetics Computer Group (GCG), Madison, Wis.), and functions available through the Vector NTI 5.5 suite of programs (Informax, Bethesda, Md.).

TABLE 6
DNA Sequencing Primers

| DmGP | | | Internal Primers | |
|---|---|---|---|---|
| CR | 5′ Primer | 3′ Primer | Sense | Antisense |
| 1 | VGS28-gtagccgccATGGCC | VGS29-gtaTCAGTTGATT | VGS49-TGCAGCATCTAC | VGS50-GATTGGCG |
| | AACTTAAGCTGGCTGA | CGCCTCCCCAGCTCT | ATATCCACGCTGA (SEQ | ACACGGCACCCGT |
| | GCAC (SEQ ID NO:184) | (SEQ ID NO:185) | ID NO:186) | GCCA (SEQ ID |
| | | | | NO:187) |
| 2 | VGS30-gtagccgccATGTCA | VGS31gtaTTACCGCGGC | VGS59-GTACGGCGTGCT | VGS60-ATTGCGAG |
| | CTACCCAGCTGGCTAAC | ATCAGCTTGGTGACC | AATCGTCTTCGGC (SEQ | CAGTGCGCATGAT |
| | AGA | (SEQ ID NO:190) | ID NO:191) | GGGC (SEQ ID |
| | (SEQ ID NO:188) | | | NO:192) |
| | DEL1937-gccgccATGAAT | | | |
| | CAGACGGAGCCCGCCC | | | |
| | AGC | | | |
| | (SEQ ID NO:189) | | | |
| 3 | DEL1840-gccgccATGTCG | DEL1860-TTCCAGTGGC | VGS65-ATGTGGCCAGAT | VGS66-CAATCATG |
| | GAGATTGTCGACACCG | AGGACAGATCGGGAT | GGACGATATCCCA (SEQ | GGAATGCCCGTAG |
| | AGC (SEQ ID NO:193) | (SEQ ID NO:194) | ID NO:195) | TCAG (SEQ ID |
| | | | | NO:196) |
| 4 | DEL1933-gccgccATGGAG | DEL1934-TTAGAGTCCA | VGS47-GCCATCATCCGG | VGS48-AATGGGAT |
| | AACACCACAATGCTGG | GTGGTGGAGGTCCTG | CCACTGCAGCCGC (SEQ | TGTACATGGAGTT |
| | CTA (SEQ ID NO:197) | (SEQ ID NO:198) | ID NO:199) | GCTC (SEQ ID NO: |
| | | | | 200) |
| 5 | DEL1844-gccgccATGGAG | DEL1845-tctagaTCAGGAG | DEL1891-ATCTCCATCG | DEL1892-GCCGCGA |
| | AATCGCAGTGACTTCG | AGCAGTTGGGTGGTGTT | ACAGATACGT (SEQ ID | TGGCCAGGTTGCA |
| | AGGC (SEQ ID NO:201) | GGC (SEQ ID NO:202) | NO:203) | (SEQ ID NO:204) |
| 6 | DEL1842-gccgccATGTAC | DEL1862-CGATCGGCGC | VGS51-GTCACCAATTAC | VGS52-GGGCAGCC |
| | TACATAGCTCACCAGC | ACCGGAGAATCAGTT | TTTATAGCCAGCT (SEQ | AACAGCAGGTGAA |
| | AGCCG (SEQ ID NO:205) | (SEQ ID NO:207) | ID NO:209) | CACA (SEQ ID |
| | DEL1990-gccgccATGGAG | DEL1989-TCAAAACTCG | | NO:210) |
| | CACCACAATAGCCATCT | GTGCTTCTTATGTTTG | | DEL1991-GTGAGAT |
| | GTT (SEQ ID NO:206) | (SEQ ID NO:208) | | GACTACGAAGTAC |
| | | | | CATC (SEQ ID |
| | | | | NO:211) |
| 7 | VGS69-gtagccgccATGGCA | VGS70-TTAAAGTGGTTG | VGS74-GGGCACACGTG | VGS73-ATAGAGCT |
| | ATGGACTTAATCGAGC | CCACAAGGACT (SEQ ID | CTCCTGGTAACG (SEQ | GCAGTGGCAGCCA |
| | A (SEQ ID NO:212) | NO:213) | ID NO:214) | GC (SEQ ID NO:215) |
| 8 | VGS38-gtagccgccATGTTT | VGS39-gtaATTACAAATC | VGS55-GTGCAAAGCCT | VGS56-TGAGTATTT |
| | ACGTGGCTGATGATGG | TGTCTGCTGCACTGCG | ACATGGTGAGCACA | CCAGTCGGGAGAG |
| | ATGT (SEQ ID NO: 216) | (SEQ ID NO:217) | (SEQ ID NO:218) | GTC (SEQ ID |
| | | | | NO:219) |
| 9 | VGS40-gtagccgccATGTTC | VGS41-gtaTTAGAGCTGA | VGS53-GTGCTCTGCATG | VGS54-GACGAACA |
| | AACTACGAGGAGGGGG | GGACTGTTGACGGCG | CCCGTCACCCTGG (SEQ | GCATCTTGACCAC |
| | ATGC (SEQ ID NO:220) | (SEQ ID NO:221) | ID NO:222) | ACGC (SEQ ID |
| | | | | NO:223) |
| 11 | DEL1905-gccgccATGGCT | DEL1906-TTAGAGCATT | VGS57-CCCGTGACTAGC | VGS58-ACCGGAAT |
| | GGCCATCAGTCGCTGG | TCAATATTGGACGTT | ATGTCCCTGCGAA (SEQ | CGCAGTCGTCACA |
| | CAC (SEQ ID NO:224) | (SEQ ID NO:225) | ID NO:226) | ATCG (SEQ ID |
| | | | | NO:227) |

The results of cloning and sequencing of the DmGPCRs of the present invention follows:

DmGPCR1

PCR primers designed to the cDNA corresponding to PnuFlyPep34651 were used to successfully amplify a PCR product from a cDNA preparation prepared from Drosophila polyA⁺ mRNA. The resulting product was cloned and sequenced. The experimentally obtained sequence was identical to the predicted sequence. An intact clone was obtained and designated ‘DmGPCR1.’

DmGPCR2

Initial attempts to amplify a PCR product using primers designed to the cDNA corresponding to PnuFlyPep67585 were unsuccessful. Alignment of the predicted sequence to the existing C. elegans receptors, and to other neuropeptide receptors, showed that the 5′ end of the predicted sequence was unusually long, and suggested that there may have been an error in gene prediction on that side. Using the genomic sequence as a guide, a variety of alternative 5′ PCR primers were designed and tested. One of these primer combinations, using cDNA prepared from total RNA, was successful in giving a product of the right size. Sequencing of clones derived from the PCR reaction showed that the amplified product contained the anticipated 5′ and 3′ ends, and was identical to the predicted sequence with the exception that the predicted sequence was missing a small stretch of 6 amino acids. Comparison of the clones also revealed that two splicing isoforms were present, one similar to the predicted sequence (designated ‘DmGPCR2a’), and the other missing a stretch of 23 amino acids located just past TM VII into the intracellular C-terminus of the molecule (designated DmGPCR2b′).

DmGPCR3

A gene corresponding to the DmGPCR3 predicted protein had already been reported in the literature. This gene (GenBank accession M77168) was described as NKD, “a developmentally regulated tachykinin receptor”. Monnier D, et al., J. Biol. Chem. 1992, 267(2), 1298-302. Comparison of the M77168 and PnuFlyPep68505 sequences showed that the predicted sequences were significantly different from the cDNA. The cDNA had a longer 5′ end, was missing an exon encoding 51 amino acids, and was significantly shorter on the 3′ end. PCR primers were designed to the published sequence, and a PCR product was obtained using cDNA prepared from total RNA. This product was identical in structure to the reported NKD sequence.

DmGPCR4

The cDNA corresponding to PnuFlyPep 67393 was used to design PCR primers for the amplification of DmGPCR4. Using a cDNA library prepared from total Drosophila mRNA, a PCR product was obtained and cloned. Comparison of the clones with the sequence predicted by PnuFlyPep revealed that the sequences were identical with the exception that one exon predicted by HMMGene was not present in any of the cloned PCR products. DmGPCR4 has been recently cloned by Lenz et al., Biochem. Biophys. Res. Comm., 2000, 273, 571-577, and was classified as a second putative allatostatin receptor.

DmGPCR5

DmGPCR5 (FlyPepCG7887) incorrectly contains a frameshift mutation. The PnuFlyPep version, PnuFlyPep67522, which has been described in the literature as a ‘Drosophila receptor for tachykinin-related peptides’ (M77168) (Li X J, et al., EMBO Journal, 1991, 10(11), 3221-3229), corrects that mistake but incorrectly predicts some internal sequences and the C-terminus. At first appearance, the predicted cDNA corresponding to the PnuFlyPep protein was identical to the published sequence. PCR primers were used to successfully amplify a PCR product of the appropriate size from a cDNA mixture prepared from Drosophila melanogaster poly A⁺ mRNA. Sequencing of the cloned PCR products revealed that, although the overall splicing pattern was the same, two sequencing errors were present in the PnuFlyPep sequence. These errors resulted in a frameshift mutation followed by a compensatory frameshift mutation, resulting in a difference of 13 amino acids between the experimentally determined and reported sequences, starting at amino acid position 46. This cloned gene was designated ‘DmGPCR5a.’

Additionally, a splicing isoform was found for DmGPCR5. This variant encoded an extra three amino acids in the N-terminal extracellular domain. This variant was designated ‘DmGPCR5b’.

DmGPCR6

The GPCR corresponding to PnuFlyPep 15731 had already been described in the literature as a ‘Neuropeptide Y’ receptor (M81490. Li X J, et al., J. Biol. Chem., 1992, 267(1), 9-12). The PnuFlyPep-predicted sequence was different from M81490 at both ends of the molecule. PnuFlyPepl5731 contained an extra 15 amino acids on the N-terminus as compared to M81490. The 3′ end of PnuFlyPep 15731 was also different from M81490, being truncated and not containing conserved TM VI and TM VII residues.

The initial PCR primers were designed using the sequence of M81490. Using these primers, and a template derived from total mRNA, a PCR product was obtained. Examination of the cloned PCR product revealed that it used an identical processing pattern to M81490. This clone was designated ‘DmGPCR6a’.

During the cloning of DmGPCR6a an additional splicing isoform was discovered. This isoform was generated by use of an alternative splice acceptor site to generate an alternative 3′ end of the molecule using much of the same sequence as the ‘6a’ form but in a different reading frame. Additionally, the open reading frame for this clone extended past the original 3′ PCR primer. Examination of the genomic sequence on the 3′ end revealed a number of likely candidate exons. PCR primers corresponding to a number of these possible exons were tested until one was found that would amplify a PCR product. This product was designated ‘6b’. Examination of the genomic sequence also predicted that the initiator ATG predicted by PnuFlyPepl5731 was in-frame with the M81490 initiation codon containing an extra 15 amino acids, and that it was likely that the PnuFlyPep 15731 start codon was the authentic start codon. A new 5′ PCR primer was designed that incorporated the PnuFlyPep 15731 start codon and was used in conjunction with the two 3′ PCR primers to amplify and clone ‘DmGPCR6aL’ and ‘DmGPCR6bL’ (‘long’).

DmGPCR7

Initial attempts to amplify the DmGPCR7 gene product were unsuccessful. Alignment of the predicted sequence (PnuFlyPep67863) with other GPCRs suggested that the error was probably in the prediction of the 3′ end of the molecule. The predicted sequence had a 3′ end that was far longer than that of most other GPCRs. Examination of the genomic sequence suggested that the likely error was in the prediction of a splicing event that removed an in-frame stop codon that would have resulted in a molecule of the appropriate size. A 3′ PCR primer was designed within that intron. Additionally, a new 5′ PCR primer was designed to utilize an in-frame ATG just upstream of the predicted start codon. PCR amplification of cDNA derived from total mRNA resulted in a product of the expected size.

The PnuFlyPep and WO 01/70980 versions of DmGPCR7 are both missing two amino acids on the N-terminus. As previously noted, the PnuFlyPep and FlyPep CG10626 versions are also incorrect at the C-terminus. The incorrect versions of the DmGPCR7 gene product were predicted to be putative Drosophila leucokinin receptors (e.g., Hewes & Taghert, Genome Res., 2001, 11, 1126-1142; Holmes et al., Insect Mol. Biol., 2000, 9, 457-465); however, no experimental evidence prior to this invention has confirmed this prediction.

DmGPCR8

DmGPCR8 was successfully amplified using PCR primers designed to the PnuFlyPep predicted sequence. cDNA derived from poly A⁺ RNA was used as template for the PCR reaction. All six of the sequenced clones were identical in structure to the PnuFlyPep-predicted sequence. A polymorphism was noted at position #68 (DNA sequence), with half of the clones having a “C” at this position, and half an “A.” This change does result in an amino acid change, Asp or Glu, respectively. The Celera sequence noted an “A,” so an “A” clone (Glu) was arbitrarily chosen for further study. No “A” clones were obtained in the correct orientation, thus a subcloning step, utilizing Pme I to remove the insert from the original pCR3.1 clone and a Pme I-digested pCR3.1 vector, was used to reverse the orientation.

The PnuFlyPep version is correct. The WO 01/70980 version, however, is missing approximately 17 N-terminal amino acids and approximately 15 internal amino acids. This receptor was classified as a putative somatostatin-like receptor (e.g., Hewes & Taghert, Genome Res., 2001, 11, 1126-1142). No experimental evidence prior to this invention has confirmed this prediction.

DmGPCR9

DmGPCR9 was cloned using PCR primers designed to the PnuFlyPep predicted sequence and a cDNA template prep prepared from poly A⁺ RNA. The genomic structure was correctly predicted in PnuFlyPep.

DmGPCR10

Initial attempts to generate a PCR product with primers designed for DmGPCR10 (PnuFlyPep7O325) were unsuccessful. Examination of the predicted cDNA showed that the predicted sequence was unusual in that it did not contain the highly conserved “WXP” motif in TM VI, nor the “NPXXF” motif in TM VII, though several other conserved residues were present. Examination of genomic sequences up to 80 kb downstream of the last exon did not reveal any other potential exons. Attempts to obtain an intact clone for DmGPCR10 were not undertaken.

DmGPCR1 (Allatostatin-Like Peptide Receptor)

PCR primers for the ‘allatostatin-like peptide receptor were designed using the published sequence. Birgul et al., EMBO Journal, 1999, 18(21), 5892-5900. A PCR product was obtained using cDNA derived from a total mRNA prep, and was cloned and sequenced. The final cDNA coded for a protein identical to that described in publication.

### Example 3

### Northern Blot Analysis

Northern blots may be performed to examine the expression of mRNA. The sense orientation oligonucleotide and the antisense-orientation oligonucleotide, described above, are used as primers to amplify a portion of the GPCR cDNA sequence of a nucleotide sequence selected from the group consisting of SEQ ID NO: 1, 3, 5, 7, 9, 11, 13, 15, 17, 19, 21, and 23.

Multiple human tissue northern blot from Clontech (Human II # 7767-1) are hybridized with the probe. Pre-hybridization is carried out at 42° C. for 4 hours in 5×SSC, 1× Denhardt's reagent, 0.1% SDS, 50% formamide, 250 mg/ml salmon sperm DNA. Hybridization is performed overnight at 42° C. in the same mixture with the addition of about 1.5×10⁶ cpm/ml of labeled probe.

The probe is labeled with α-32P-dCTP by Rediprime DNA labelling system (Amersham Pharmacia), purified on Nick Column (Amersham Pharmacia), and added to the hybridization solution. The filters are washed several times at 42° C. in 0.2×SSC, 0.1% SDS. Filters are exposed to Kodak XAR film (Eastman Kodak Company, Rochester, N.Y., USA) with intensifying screen at −80° C.

### Example 4

### Recombinant Expression of DmGPCR in Eukaryotic Cells

Expression of DmGPCR in Mammalian Cells

To produce DmGPCR protein, a DmGPCR-encoding polynucleotide is expressed in a suitable host cell using a suitable expression vector and standard genetic engineering techniques. For example, the DmGPCR-encoding sequence described in Example 1 is subcloned into the commercial expression vector pzeoSV2 (Invitrogen, San Diego, Calif.) and transfected into Chinese Hamster Ovary (CHO) cells using the transfection reagent FuGENE 6 (Boehringer-Mannheim) and the transfection protocol provided in the product insert. Other eukaryotic cell lines, including human embryonic kidney (HEK 293) and COS cells, for example, are suitable as well. Cells stably expressing DmGPCR are selected by growth in the presence of 100 μg/ml zeocin (Stratagene, LaJolla, Calif.). Optionally, DmGPCR may be purified from the cells using standard chromatographic techniques. To facilitate purification, antisera is raised against one or more synthetic peptide sequences that correspond to portions of the DmGPCR amino acid sequence, and the antisera is used to affinity purify DmGPCR. The DmGPCR also may be expressed in-frame with a tag sequence (e.g., polyhistidine, hemagluttinin, FLAG) to facilitate purification. Moreover, it will be appreciated that many of the uses for DmGPCR polypeptides, such as assays described below, do not require purification of DmGPCR from the host cell.

Expression of DmGPCR in 293 Cells

For expression of DmGPCR in 293 cells, a plasmid bearing the relevant DmGPCR coding sequence is prepared, using vector pSecTag2A (Invitrogen). Vector pSecTag2A contains the murine IgK chain leader sequence for secretion, the c-myc epitope for detection of the recombinant protein with the anti-myc antibody, a C-terminal polyhistidine for purification with nickel chelate chromatography, and a Zeocin resistant gene for selection of stable transfectants. The forward primer for amplification of this GPCR cDNA is determined by routine procedures and preferably contains a 5′ extension of nucleotides to introduce the HindIII cloning site and nucleotides matching the GPCR sequence. The reverse primer is also determined by routine procedures and preferably contains a 5′ extension of nucleotides to introduce an XhoI restriction site for cloning and nucleotides corresponding to the reverse complement of the DmGPCR sequence. The PCR conditions are 55° C. as the annealing temperature. The PCR product is gel purified and cloned into the HindIII-XhoI sites of the vector.

The DNA is purified using Qiagen chromatography columns and transfected into 293 cells using DOTAP transfection media (Boehringer Mannheim, Indianapolis, Ind.). Transiently transfected cells are tested for expression after 24 hours of transfection, using western blots probed with antiHis and anti-DmGPCR peptide antibodies. Permanently transfected cells are selected with Zeocin and propagated. Production of the recombinant protein is detected from both cells and media by western blots probed with anti-His, anti-Myc, or anti-GPCR peptide antibodies.

Expression of DmGPCR in COS Cells

For expression of the DmGPCR in COS7 cells, a polynucleotide molecule having a nucleotide sequence selected from the group consisting of SEQ ID NOs: 1, 3, 5, 7, 9, 11, 13, 15, 17, 19, 21, or 23 can be cloned into vector p3-Cl. This vector is a pUCI 8-derived plasmid that contains the HCMV (human cytomegalovirus) promoter-intron located upstream from the bGH (bovine growth hormone) polyadenylation sequence and a multiple cloning site. In addition, the plasmid contains the dhfr (dihydrofolate reductase) gene which provides selection in the presence of the drug methotrexane (MTX) for selection of stable transformants.

The forward primer is determined by routine procedures and preferably contains a 5′ extension which introduces an XbaI restriction site for cloning, followed by nucleotides which correspond to a nucleotide sequence selected from the group consisting of SEQ ID NOs: 1, 3, 5, 7, 9, 11, 13, 15, 17, 19, 21, or 23. The reverse primer is also determined by routine procedures and preferably contains 5′-extension of nucleotides which introduces a SalI cloning site followed by nucleotides which correspond to the reverse complement of a nucleotide sequence selected from the group consisting of SEQ ID NOs: 1, 3, 5, 7, 9, 11, 13, 15, 17, 19, 21, or 23.

The PCR consists of an initial denaturation step of 5 min at 95° C., 30 cycles of 30 sec denaturation at 95° C., 30 sec annealing at 58° C., and 30 sec extension at 72° C., followed by 5 min extension at 72° C. The PCR product is gel purified and ligated into the XbaI and SalI sites of vector p3-CI. This construct is transformed into E. coli cells for amplification and DNA purification. The DNA is purified with Qiagen chromatography columns and transfected into COS7 cells using Lipofectamine reagent from BRL, following the manufacturer's protocols. Forty eight and 72 hours after transfection, the media and the cells are tested for recombinant protein expression.

DmGPCR expressed from a COS cell culture can be purified by concentrating the cell-growth media to about 10 mg of protein/ml, and purifying the protein by, for example, chromatography. Purified DmGPCR is concentrated to 0.5 mg/ml in an Amicon concentrator fitted with a YM-10 membrane and stored at −80° C.

Expression of DmGPCR in Insect Cells

For expression of DmGPCR in a baculovirus system, a polynucleotide molecule having a nucleotide sequence selected from the group consisting of SEQ ID NOs: 1, 3, 5, 7, 9, 11, 13, 15, 17, 19, 21, or 23 can be amplified by PCR. The forward primer is determined by routine procedures and preferably contains a 5′ extension which adds the NdeI cloning site, followed by nucleotides which correspond to a nucleotide sequence selected from the group consisting of SEQ ID NOs: 1, 3, 5, 7, 9, 11, 13, 15, 17, 19, 21, or 23. The reverse primer is also determined by routine procedures and preferably contains a 5′ extension which introduces the KpnI cloning site, followed by nucleotides which correspond to the reverse complement of a nucleotide sequence selected from the group consisting of SEQ ID NOs: 1, 3, 5, 7, 9, 11, 13, 15, 17, 19, 21, or 23.

The PCR product is gel purified, digested with NdeI and KpnI, and cloned into the corresponding sites of vector pAcHTL-A (Pharmingen, San Diego, Calif.). The pAcHTL expression vector contains the strong polyhedrin promoter of the Autographa californica nuclear polyhedrosis virus (AcMNPV), and a 6×His tag upstream from the multiple cloning site. A protein kinase site for phosphorylation and a thrombin site for excision of the recombinant protein precedes the multiple cloning site is also present. Of course, many other baculovirus vectors could be used in place of pAcHTL-A, such as pAc373, pVL941 and pAcIM1. Other suitable vectors for the expression of GPCR polypeptides can be used, provided that the vector construct includes appropriately located signals for transcription, translation, and trafficking, such as an in-frame AUG and a signal peptide, as required. Such vectors are described in Luckow et al., Virology 170:31-39, among others.

The virus is grown and isolated using standard baculovirus expression methods, such as those described in Summers et al. (A MANUAL OF METHODS FOR BACULOVIRUS VECTORS AND INSECT CELL CULTURE PROCEDURES, Texas Agricultural Experimental Station Bulletin No. 1555 (1987)).

In one embodiment, pAcHLT-A containing DmGPCR gene is introduced into baculovirus using the “BaculoGold” transfection kit (Pharmingen, San Diego, Calif.) using methods established by the manufacturer. Individual virus isolates are analyzed for protein production by radiolabeling infected cells with ³⁵S-methionine at 24 hours post infection. Infected cells are harvested at 48 hours post infection, and the labeled proteins are visualized by SDS-PAGE. Viruses exhibiting high expression levels can be isolated and used for scaled up expression.

For expression of a DmGPCR polypeptide in Sf9 cells, a polynucleotide molecule having a nucleotide sequence selected from the group consisting of SEQ ID NOs: 1, 3, 5, 7, 9, 11, 13, 15, 17, 19, 21, or 23, can be amplified by PCR using the primers and methods described above for baculovirus expression. The DmGPCR cDNA is cloned into vector pAcHLT-A (Pharmingen) for expression in Sf9 insect cells. The insert is cloned into the NdeI and KpnI sites, after elimination of an internal NdeI site (using the same primers described above for expression in baculovirus). DNA is purified with Qiagen chromatography columns and expressed in Sf9 cells. Preliminary Western blot experiments from non-purified plaques are tested for the presence of the recombinant protein of the expected size which reacted with the GPCR-specific antibody. These results are confirmed after further purification and expression optimization in HiG5 cells.

### Example 5

### Interaction Trap/Two-Hybrid System

In order to assay for DmGPCR-interacting proteins, the interaction trap/two-hybrid library screening method can be used. This assay was first described in Fields, et al., Nature, 1989, 340, 245, which is incorporated herein by reference in its entirety. A protocol is published in CURRENT PROTOCOLS IN MOLECULAR BIOLOGY, John Wiley & Sons, NY, 1999, and Ausubel et al., SHORT PROTOCOLS IN MOLECULAR BIOLOGY, fourth edition, Greene and Wiley-interscience, NY, 1992, which are incorporated herein by reference in their entireties. Kits are available from Clontech, Palo Alto, Calif. (Matchmaker Two-Hybrid System 3).

A fusion of the nucleotide sequences encoding all or partial DmGPCR and the yeast transcription factor GAL4 DNA-binding domain (DNA-BD) is constructed in an appropriate plasmid (i.e., pGBKT7) using standard subcloning techniques. Similarly, a GAL4 active domain (AD) fusion library is constructed in a second plasmid (i.e., pGADT7) from cDNA of potential GPCR-binding proteins (for protocols on forming cDNA libraries, see Sambrook et al., MOLECULAR CLONING: A LABORATORY MANUAL, second edition, Cold Spring Harbor Press, Cold Spring Harbor, N.Y., 1989), which is incorporated herein by reference in its entirety. The DNA-BD/GPCR fusion construct is verified by sequencing, and tested for autonomous reporter gene activation and cell toxicity, both of which would prevent a successful two-hybrid analysis. Similar controls are performed with the AD/library fusion construct to ensure expression in host cells and lack of transcriptional activity. Yeast cells are transformed (ca. 105 transformants/mg DNA) with both the GPCR and library fusion plasmids according to standard procedure (Ausubel et al., SHORT PROTOCOLS IN MOLECULAR BIOLOGY, fourth edition, Greene and Wiley-interscience, NY, 1992, which is incorporated herein by reference in its entirety). In vivo binding of DNA-BD/GPCR with AD/library proteins results in transcription of specific yeast plasmid reporter genes (i.e., lacZ, HIS3, ADE2, LEU2). Yeast cells are plated on nutrient-deficient media to screen for expression of reporter genes. Colonies are dually assayed for β-galactosidase activity upon growth in Xgal (5-bromo-4-chloro-3-indolyl-β-D-galactoside) supplemented media (filter assay for P-galactosidase activity is described in Breeden et al., Cold Spring Harb. Symp. Quant. Biol., 1985, 50, 643, which is incorporated herein by reference in its entirety). Positive AD-library plasmids are rescued from transformants and reintroduced into the original yeast strain as well as other strains containing unrelated DNA-BD fusion proteins to confirm specific DmGPCR/library protein interactions. Insert DNA is sequenced to verify the presence of an open reading frame fused to GAL4 AD and to determine the identity of the DmGPCR-binding protein.

### Example 6

### Mobility Shift DNA-Binding Assay Using Gel Electrophoresis

A gel electrophoresis mobility shift assay can rapidly detect specific protein-DNA interactions. Protocols are widely available in such manuals as Sambrook et al., MOLECULAR CLONING: A LABORATORY MANUAL, second edition, Cold Spring Harbor Press, Cold Spring Harbor, N.Y., 1989, and Ausubel et al., SHORT PROTOCOLS IN MOLECULAR BIOLOGY, fourth edition, Greene and Wiley-interscience, NY, 1992, each of which is incorporated herein by reference in its entirety.

Probe DNA(<300 bp) is obtained from synthetic oligonucleotides, restriction endonuclease fragments, or PCR fragments and end-labeled with ³²P An aliquot of purified DmGPCR (ca. 15 μg) or crude DmGPCR extract (ca. 15 ng) is incubated at constant temperature (in the range 22-37° C.) for at least 30 minutes in 10-15 μl of buffer (i.e., TAE or TBE, pH 8.0-8.5) containing radiolabeled probe DNA, nonspecific carrier DNA (ca. 1 μg), BSA (300 μg/ml), and 10% (v/v) glycerol. The reaction mixture is then loaded onto a polyacrylamide gel and run at 30-35 mA until good separation of free probe DNA from protein-DNA complexes occurs. The gel is then dried and bands corresponding to free DNA and protein-DNA complexes are detected by autoradiography.

### Example 7

### Antibodies to DmGPCR

Standard techniques are employed to generate polyclonal or monoclonal antibodies to the DmGPCR and to generate useful antigen-binding fragments thereof or variants thereof, including “humanized” variants. Such protocols can be found, for example, in Sambrook et al. (1989), supra, and Harlow et al. (Eds.), ANTIBODIES: A LABORATORY MANUAL, Cold Spring Harbor Laboratory, Cold Spring Harbor, N.Y., 1988. In one embodiment, recombinant DmGPCR polypeptides (or cells or cell membranes containing such polypeptides) are used as antigen to generate the antibodies. In another embodiment, one or more peptides having amino acid sequences corresponding to an immunogenic portion of DmGPCR (e.g., 6, 7, 8, 9, 10, 11, 12, 13, 14, 15, 16, 17, 18, 19, 20, or more amino acids) are used as antigen. Peptides corresponding to extracellular portions of DmGPCR, especially hydrophilic extracellular portions, are included in the invention. The antigen may be mixed with an adjuvant or linked to a hapten to increase antibody production.

Polyclonal or Monoclonal Antibodies

As one exemplary protocol, recombinant DmGPCR or a synthetic fragment thereof is used to immunize a mouse for generation of monoclonal antibodies (or larger mammal, such as a rabbit, for polyclonal antibodies). To increase antigenicity, peptides are conjugated to Keyhole Lympet Hemocyanin (Pierce), according to the manufacturer's recommendations. For an initial injection, the antigen is emulsified with Freund's Complete Adjuvant and injected subcutaneously. At intervals of two to three weeks, additional aliquots of DmGPCR antigen are emulsified with Freund's Incomplete Adjuvant and injected subcutaneously. Prior to the final booster injection, a serum sample is taken from the immunized mice and assayed by western blot to confirm the presence of antibodies that immunoreact with DmGPCR. Serum from the immunized animals may be used as a polyclonal antisera or used to isolate polyclonal antibodies that recognize DmGPCR. Alternatively, the mice are sacrificed and their spleen removed for generation of monoclonal antibodies.

To generate monoclonal antibodies, the spleens are placed in 10 ml serum-free RPMI 1640, and single cell suspensions are formed by grinding the spleens in serum-free RPMI 1640, supplemented with 2 mM L-glutamine, 1 mM sodium pyruvate, 100 units/ml penicillin, and 100 μg/ml streptomycin (RPMI) (Gibco, Canada). The cell suspensions are filtered and washed by centrifugation and resuspended in serum-free RPMI. Thymocytes taken from three naive Balb/c mice are prepared in a similar manner and used as a Feeder Layer. NS-1 myeloma cells, kept in log phase in RPMI with 10% fetal bovine serum (FBS) (Hyclone Laboratories, Inc., Logan, Utah) for three days prior to fusion, are centrifuged and washed as well.

To produce hybridoma fusions, spleen cells from the immunized mice are combined with NS-1 cells and centrifuged, and the supernatant is aspirated. The cell pellet is dislodged by tapping the tube, and 2 ml of 37° C. PEG 1500 (50% in 75 mM HEPES, pH 8.0) (Boehringer-Mannheim) is stirred into the pellet, followed by the addition of serum-free RPMI. Thereafter, the cells are centrifuged, resuspended in RPMI containing 15% FBS, 100 μM sodium hypoxanthine, 0.4 μM aminopterin, 16 μM thymidine (HAT) (Gibco), 25 units/ml IL-6 (Boehringer-Mannheim), and 1.5×10⁶ thymocytes/ml, and plated into 10 Coming flat-bottom 96-well tissue culture plates (Corning, Corning N.Y.).

On days 2, 4, and 6 after the fusion, 100 μl of medium is removed from the wells of the fusion plates and replaced with fresh medium. On day 8, the fusions are screened by ELISA, testing for the presence of mouse IgG that binds to DmGPCR. Selected fusion wells are further cloned by dilution until monoclonal cultures producing anti-DmGPCR antibodies are obtained.

Humanization of Anti-DmGPCR Monoclonal Antibodies

The expression pattern of DmGPCR as reported herein and the proven track record of GPCRs as targets for therapeutic intervention suggest therapeutic indications for DmGPCR inhibitors (antagonists). DmGPCR-neutralizing antibodies comprise one class of therapeutics useful as DmGPCR antagonists. Following are protocols to humanize the monoclonal antibodies of the invention.

The principles of humanization have been described in the literature and are facilitated by the modular arrangement of antibody proteins. To minimize the possibility of binding complement, a humanized antibody of the IgG4 isotype may be used.

For example, a level of humanization is achieved by generating chimeric antibodies comprising the variable domains of non-human antibody proteins of interest with the constant domains of human antibody molecules. (See, e.g., Morrison et al., Adv. Immunol., 1989, 44, 65-92). The variable domains of DmGPCR-neutralizing anti-DmGPCR antibodies are cloned from the genomic DNA of a B-cell hybridoma or from cDNA generated from mRNA isolated from the hybridoma of interest. The V region gene fragments are linked to exons encoding human antibody constant domains, and the resultant construct is expressed in suitable mammalian host cells (e.g., myeloma or CHO cells).

To achieve an even greater level of humanization, only those portions of the variable region gene fragments that encode antigen-binding complementarity determining regions (“CDR”) of the non-human monoclonal antibody genes are cloned into human antibody sequences. (See, e.g., Jones et al., Nature, 1986, 321, 522-525; Riechmann et al., Nature, 1988, 332, 323-327; Verhoeyen et al., Science, 1988, 239, 1534-36; and Tempest et al., Bio/Technology, 1991, 9, 266-71). If necessary, the β-sheet framework of the human antibody surrounding the CDR3 regions also is modified to more closely mirror the three dimensional structure of the antigen-binding domain of the original monoclonal antibody. (See Kettleborough et al., Protein Engin., 1991, 4, 773-783; and Foote et al., J. Mol. Biol., 1992, 224, 487-499).

In an alternative approach, the surface of a non-human monoclonal antibody of interest is humanized by altering selected surface residues of the non-human antibody, e.g., by site-directed mutagenesis, while retaining all of the interior and contacting residues of the non-human antibody. See Padlan, Molecular Immunol., 1991, 28(4/5), 489-98.

The foregoing approaches are employed using DmGPCR-neutralizing anti-DmGPCR monoclonal antibodies and the hybridomas that produce them to generate humanized DmGPCR-neutralizing antibodies useful as therapeutics to treat or palliate conditions wherein DmGPCR expression or ligand-mediated DmGPCR signaling is detrimental.

### Example 8

### Assays to Identify Modulators of DmGPCR Activity

Set forth below are several nonlimiting assays for identifying modulators (agonists and antagonists) of DmGPCR activity. Among the modulators that can be identified by these assays are natural ligand compounds of the receptor; synthetic analogs and derivatives of natural ligands; antibodies, antibody fragments, and/or antibody-like compounds derived from natural antibodies or from antibody-like combinatorial libraries; and/or synthetic compounds identified by high-throughput screening of libraries; and the like. All modulators that bind DmGPCR are useful for identifying DmGPCR in tissue samples (e.g., for diagnostic purposes, pathological purposes, and the like). Agonist and antagonist modulators are useful for up-regulating and down-regulating DmGPCR activity, respectively, to treat disease states characterized by abnormal levels of DmGPCR activity. The assays may be performed using single putative modulators, and/or may be performed using a known agonist in combination with candidate antagonists (or visa versa).

cAMP Assays

In one type of assay, levels of cyclic adenosine monophosphate (cAMP) are measured in DmGPCR-transfected cells that have been exposed to candidate modulator compounds. Protocols for cAMP assays have been described in the literature. (See, e.g., Sutherland et al., Circulation, 1968, 37, 279; Frandsen et al., Life Sciences, 1976, 18, 529-541; Dooley et al., J. Pharm. and Exper. Ther., 1997, 283(2), 735-41; and George et al., J. Biomolecular Screening, 1997, 2(4), 235-40). An exemplary protocol for such an assay, using an Adenylyl Cyclase Activation FlashPlate® Assay from NEN™ Life Science Products, is set forth below.

Briefly, the DmGPCR coding sequence (e.g., a cDNA or intronless genomic DNA) is subcloned into a commercial expression vector, such as pzeoSV2 (Invitrogen), and transiently transfected into Chinese Hamster Ovary (CHO) cells using known methods, such as the transfection protocol provided by Boehringer-Mannheim when supplying the FuGENE 6 transfection reagent. Transfected CHO cells are seeded into 96-well microplates from the FlashPlate® assay kit, which are coated with solid scintillant to which antisera to cAMP has been bound. For a control, some wells are seeded with wild type (untransfected) CHO cells. Other wells in the plate receive various amounts of a cAMP standard solution for use in creating a standard curve.

One or more test compounds (i.e., candidate modulators) are added to the cells in each well, with water and/or compound-free medium/diluent serving as a control or controls. After treatment, cAMP is allowed to accumulate in the cells for exactly 15 minutes at room temperature. The assay is terminated by the addition of lysis buffer containing [¹²⁵I]-labeled cAMP, and the plate is counted using a Packard Topcount™ 96-well microplate scintillation counter. Unlabeled cAMP from the lysed cells (or from standards) and fixed amounts of [¹²⁵]-cAMP compete for antibody bound to the plate. A standard curve is constructed, and cAMP values for the unknowns are obtained by interpolation. Changes in intracellular cAMP levels of cells in response to exposure to a test compound are indicative of DmGPCR modulating activity. Modulators that act as agonists of receptors which couple to the Gₛ subtype of G proteins will stimulate production of cAMP, leading to a measurable 3-10 fold increase in cAMP levels. Agonists of receptors which couple to the G_{i/o} subtype of G proteins will inhibit forskolin-stimulated cAMP production, leading to a measurable decrease in cAMP levels of 50-100%. Modulators that act as inverse agonists will reverse these effects at receptors that are either constitutively active or activated by known agonists.

Aequorin Assays

In another assay, cells (e.g., CHO cells) are transiently co-transfected with both a DmGPCR expression construct and a construct that encodes the photoprotein apoaquorin. In the presence of the cofactor coelenterazine, apoaquorin will emit a measurable luminescence that is proportional to the amount of intracellular (cytoplasmic) free calcium. (See generally, Cobbold, et al., “Aequorin measurements of cytoplasmic free calcium,” in CELLULAR CALCIUM: A PRACTICAL APPROACH, McCormack J. G. and Cobbold P. H., eds., Oxford: IRL Press, 1991; Stables et al., Anal. Biochem., 1997, 252, 115-26; and Haugland, HANDBOOK OF FLUORESCENT PROBES AND RESEARCH CHEMICALS, sixth edition, Eugene, Oreg., Molecular Probes, 1996).

In one exemplary assay, DmGPCR is subcloned into the commercial expression vector pzeoSV2 (Invitrogen) and transiently co-transfected along with a construct that encodes the photoprotein apoaquorin (Molecular Probes, Eugene, Oreg.) into CHO cells using the transfection reagent FuGENE 6 (Boehringer-Mannheim) and the transfection protocol provided in the product insert.

The cells are cultured for 24 hours at 37° C. in MEM (Gibco/BRL, Gaithersburg, Md.) supplemented with 10% fetal bovine serum, 2 mM glutamine, 10 U/ml penicillin, and 10 μg/ml streptomycin, at which time the medium is changed to serum-free MEM containing 5 μM coelenterazine (Molecular Probes, Eugene, Oreg.). Culturing is then continued for two additional hours at 37° C. Subsequently, cells are detached from the plate using VERSEN (Gibco/BRL), washed, and resuspended at 200,000 cells/ml in serum-free MEM.

Dilutions of candidate DmGPCR modulator compounds are prepared in serum-free MEM and dispensed into wells of an opaque 96-well assay plate at 50 μl/well. Plates are then loaded onto an MLX microtiter plate luminometer (Dynex Technologies, Inc., Chantilly, Va.). The instrument is programmed to dispense 50 μl cell suspensions into each well, one well at a time, and immediately read luminescence for 15 seconds. Dose-response curves for the candidate modulators are constructed using the area under the curve for each light signal peak. Data are analyzed with SlideWrite, using the equation for a one-site ligand, and EC₅₀ values are obtained. Changes in luminescence caused by the compounds are considered indicative of modulatory activity. Modulators that act as agonists at receptors which couple to the Gq subtype of G proteins give an increase in luminescence of up to 100-fold. Modulators that act as inverse agonists will reverse this effect at receptors that are either constitutively active or activated by known agonists.

Luciferase Reporter Gene Assay

The photoprotein luciferase provides another useful tool for assaying for modulators of DmGPCR activity. Cells (e.g., CHO cells or COS7 cells) are transiently co-transfected with both a DmGPCR expression construct (e.g., DmGPCR in pzeoSV2) and a reporter construct which includes a gene for the luciferase protein downstream from a transcription factor binding site, such as the cAMP-response element (CRE), AP-1, or NF-kappa B. Agonist binding to receptors coupled to the Gₛ subtype of G proteins leads to increases in cAMP, thereby activating the CRE transcription factor and resulting in expression of the luciferase gene. Agonist binding to receptors coupled to the Gq subtype of G protein leads to production of diacylglycerol that activates protein kinase C, which activates the AP-1 or NF-kappa B transcription factors, in turn resulting in expression of the luciferase gene. Expression levels of luciferase reflect the activation status of the signaling events. See generally, George et al., J. Biomolecular Screening, 1997, 2(4), 235-240; and Stratowa et al., Curr. Opin. Biotechnol., 1995, 6, 574-581. Luciferase activity may be quantitatively measured using, e.g., luciferase assay reagents that are commercially available from Promega (Madison, Wis.).

In one exemplary assay, CHO cells are plated in 24-well culture dishes at a density of 100,000 cells/well one day prior to transfection and cultured at 37° C. in MEM (Gibco/BRL) supplemented with 10% fetal bovine serum, 2 mM glutamine, 10 U/ml penicillin, and 10 μg/ml streptomycin. Cells are transiently co-transfected with both a DmGPCR expression construct and a reporter construct containing the luciferase gene. The reporter plasmids CRE-luciferase, AP-1-luciferase, and NF-kappaB-luciferase may be purchased from Stratagene (LaJolla, Calif.). Transfections are performed using the FuGENE 6 transfection reagent (Boehringer-Mannheim) according to the supplier's instructions. Cells transfected with the reporter construct alone are used as a control. Twenty-four hours after transfection, cells are washed once with PBS pre-warmed to 37° C. Serum-free MEM is then added to the cells either alone (control) or with one or more candidate modulators and the cells are incubated at 37° C. for five hours. Thereafter, cells are washed once with ice-cold PBS and lysed by the addition of 100 μl of lysis buffer per well from the luciferase assay kit supplied by Promega. After incubation for 15 minutes at room temperature, 15 μl of the lysate is mixed with 50 μl of substrate solution (Promega) in an opaque-white, 96-well plate, and the luminescence is read immediately on a Wallace model 1450 MicroBeta scintillation and luminescence counter (Wallace Instruments, Gaithersburg, Md.).

Differences in luminescence in the presence versus the absence of a candidate modulator compound are indicative of modulatory activity. Receptors that are either constitutively active or activated by agonists typically give a 3-20-fold stimulation of luminescence compared to cells transfected with the reporter gene alone. Modulators that act as inverse agonists will reverse this effect.

Intracellular Calcium Measurement using FLIPR

Changes in intracellular calcium levels are another recognized indicator of G protein-coupled receptor activity, and such assays can be employed to screen for modulators of DmGPCR activity. For example, CHO cells stably transfected with a DmGPCR expression vector are plated at a density of 4×10⁴ cells/well in Packard black-walled, 96-well plates specially designed to discriminate fluorescence signals emanating from the various wells on the plate. The cells are incubated for 60 minutes at 37° C. in modified Dulbecco's PBS (D-PBS) containing 36 mg/L pyruvate and 1 g/L glucose and one of four calcium indicator dyes (Fluo-3™ AM, Fluo-4™ AM, Calcium Green™-1 AM, or Oregon Green™ 488 BAPTA-1 AM), each at a concentration of 4 μM. Plates are washed once with modified D-PBS and incubated for 10 minutes at 37° C. to remove residual dye from the cellular membrane. In addition, a series of washes with modified D-PBS is performed immediately prior to activation of the calcium response.

A calcium response is initiated by the addition of one or more candidate receptor agonist compounds, calcium ionophore A23187 (10 μM; positive control), or ATP (4 μM; positive control). Fluorescence is measured by Molecular Device's FLIPR with an argon laser (excitation at 488 nm). (See, e.g., Kuntzweiler et al., Drug Dev. Res., 1998, 44(1), 14-20). The F-stop for the detector camera was set at 2.5, and the length of exposure was 0.4 milliseconds. Basal fluorescence of cells was measured for 20 seconds prior to addition of candidate agonist, ATP, or A23187, and the basal fluorescence level was subtracted from the response signal. The calcium signal is measured for approximately 200 seconds, taking readings every two seconds. Calcium ionophore A23187 and ATP increase the calcium signal 200% above baseline levels. In general, activated GPCRs increase the calcium signal approximately 10-15% above baseline signal.

Mitogenesis Assay

In a mitogenesis assay, the ability of candidate modulators to induce or inhibit DmGPCR-mediated cell division is determined. (See, e.g., Lajiness et al., J. Pharm. and Exper. Ther., 1993, 267(3), 1573-1581). For example, CHO cells stably expressing DmGPCR are seeded into 96-well plates at a density of 5000 cells/well and grown at 37° C. in MEM with 10% fetal calf serum for 48 hours, at which time the cells are rinsed twice with serum-free MEM. After rinsing, 80 μl of fresh MEM, or MEM containing a known mitogen, is added along with 20 μl MEM containing varying concentrations of one or more candidate modulators or test compounds diluted in serum-free medium. As controls, some wells on each plate receive serum-free medium alone, and some receive medium containing 10% fetal bovine serum. Untransfected cells or cells transfected with vector alone also may serve as controls.

After culture for 16-18 hours, 1 μCi of [³H]-thymidine (2 Ci/mmol) is added to the wells and cells are incubated for an additional 2 hours at 37° C. The cells are trypsinized and collected on filter mats with a cell harvester (Tomtec); the filters are then counted in a Betaplate counter. The incorporation of [³H]-thymidine in serum-free test wells is compared to the results achieved in cells stimulated with serum (positive control). Use of multiple concentrations of test compounds permits creation and analysis of dose-response curves using the non-linear, least squares fit equation: A=B×[C/(D+C)]+G, where A is the percent of serum stimulation; B is the maximal effect minus baseline; C is the EC₅₀; D is the concentration of the compound; and G is the maximal effect. Parameters B, C and G are determined by Simplex optimization.

Agonists that bind to the receptor are expected to increase [³H]-thymidine incorporation into cells, showing up to 80% of the response to serum. Antagonists that bind to the receptor will inhibit the stimulation seen with a known agonist by up to 100%.

[³⁵S]GTPγS Binding Assay

Because G protein-coupled receptors signal through intracellular G proteins whose activity involves GTP binding and hydrolysis to yield bound GDP, measurement of binding of the non-hydrolyzable GTP analog [³⁵S]GTPγS in the presence and absence of candidate modulators provides another assay for modulator activity. See, e.g., Kowal et al., Neuropharmacology, 1998, 37, 179-187.

In one exemplary assay, cells stably transfected with a DmGPCR expression vector are grown in 10 cm tissue culture dishes to subconfluence, rinsed once with 5 ml of ice-cold Ca²⁺/Mg²⁺-free phosphate-buffered saline, and scraped into 5 ml of the same buffer. Cells are pelleted by centrifugation (500×g, 5 minutes), resuspended in TEE buffer (25 mM Tris, pH 7.5, 5 mM EDTA, 5 mM EGTA), and frozen in liquid nitrogen. After thawing, the cells are homogenized using a Dounce homogenizer (one ml TEE per plate of cells), and centrifuged at 1,000×g for 5 minutes to remove nuclei and unbroken cells.

The homogenate supernatant is centrifuged at 20,000×g for 20 minutes to isolate the membrane fraction, and the membrane pellet is washed once with TEE and resuspended in binding buffer (20 mM HEPES, pH 7.5, 150 mM NaCl, 10 mM MgCl₂, 1 mM EDTA). The resuspended membranes can be frozen in liquid nitrogen and stored at −70° C. until use.

Aliquots of cell membranes prepared as described above and stored at −70° C. are thawed, homogenized, and diluted into buffer containing 20 mM HEPES, 10 mM MgCl₂, 1 mM EDTA, 120 mM NaCl, 10 μM GDP, and 0.2 mM ascorbate, at a concentration of 10-50 μg/ml. In a final volume of 90 μl, homogenates are incubated with varying concentrations of candidate modulator compounds or 100 μM GTP for 30 minutes at 30° C. and then placed on ice. To each sample, 10 μl guanosine 5′-O-(3[³⁵S]thio) triphosphate (NEN, 1200 Ci/mmol; [³⁵S]-GTPγS), was added to a final concentration of 100-200 pM. Samples are incubated at 30° C. for an additional 30 minutes, 1 ml of 10 mM HEPES, pH 7.4, 10 mM MgCl₂, at 4° C. is added and the reaction is stopped by filtration.

Samples are filtered over Whatman GF/B filters and the filters are washed with 20 ml ice-cold 10 mM HEPES, pH 7.4, 10 mM MgCl₂. Filters are counted by liquid scintillation spectroscopy. Nonspecific binding of [³⁵S]-GTPγS is measured in the presence of 100 μM GTP and subtracted from the total. Compounds are selected that modulate the amount of [³⁵S]GTPγS binding in the cells, compared to untransfected control cells. Activation of receptors by agonists gives up to a five-fold increase in [³⁵S]GTPγS binding. This response is blocked by antagonists.

MAP Kinase Activity Assay

Evaluation of MAP kinase activity in cells expressing a GPCR provides another assay to identify modulators of DmGPCR activity. See, e.g., Lajiness et al., J. Pharm. and Exper. Ther., 1993, 267(3), 1573-1581 and Boulton et al., Cell, 1991, 65, 663-675.

In one embodiment, CHO cells stably transfected with DmGPCR are seeded into 6-well plates at a density of 70,000 cells/well 48 hours prior to the assay. During this 48-hour period, the cells are cultured at 37° C. in MEM medium supplemented with 10% fetal bovine serum, 2 mM glutamine, 10 U/ml penicillin, and 10 μg/ml streptomycin. The cells are serum-starved for 1-2 hours prior to the addition of stimulants.

For the assay, the cells are treated with medium alone or medium containing either a candidate agonist or 200 nM Phorbol ester-myristoyl acetate (i.e., PMA, a positive control), and the cells are incubated at 37° C. for varying times. To stop the reaction, the plates are placed on ice, the medium is aspirated, and the cells are rinsed with 1 ml of ice-cold PBS containing 1 mM EDTA. Thereafter, 200 μl of cell lysis buffer (12.5 mM MOPS, pH 7.3, 12.5 mM glycerophosphate, 7.5 mM MgCl₂, 0.5 mM EGTA, 0.5 mM sodium vanadate, 1 mM benzamidine, 1 mM dithiothreitol, 10 μg/ml leupeptin, 10 μg/ml aprotinin, 2 μg/ml pepstatin A, and 1 μM okadaic acid) is added to the cells. The cells are scraped from the plates and homogenized by 10 passages through a 23 ¾ G needle, and the cytosol fraction is prepared by centrifugation at 20,000×g for 15 minutes.

Aliquots (5-10 μl containing 1-5 μg protein) of cytosol are mixed with 1 mM MAPK Substrate Peptide (APRTPGGRR (SEQ ID NO: 168), Upstate Biotechnology, Inc., N.Y.) and 50 μM [γ-³² P]ATP (NEN, 3000 Ci/mmol), diluted to a final specific activity of ˜2000 cpm/pmol, in a total volume of 25 μl. The samples are incubated for 5 minutes at 30° C., and reactions are stopped by spotting 20 μl on 2 cm² squares of Whatman P81 phosphocellulose paper. The filter squares are washed in 4 changes of 1% H₃PO₄, and the squares are subjected to liquid scintillation spectroscopy to quantitate bound label. Equivalent cytosolic extracts are incubated without MAPK substrate peptide, and the bound label from these samples are subtracted from the matched samples with the substrate peptide. The cytosolic extract from each well is used as a separate point. Protein concentrations are determined by a dye binding protein assay (Bio-Rad Laboratories). Agonist activation of the receptor is expected to result in up to a five-fold increase in MAPK enzyme activity. This increase is blocked by antagonists.

[³H]Arachidonic Acid Release

The activation of GPCRs also has been observed to potentiate arachidonic acid release in cells, providing yet another useful assay for modulators of GPCR activity. See, e.g., Kanterman et al., Molecular Pharmacology, 1991, 39, 364-369. For example, CHO cells that are stably transfected with a DmGPCR expression vector are plated in 24-well plates at a density of 15,000 cells/well and grown in MEM medium supplemented with 10% fetal bovine serum, 2 mM glutamine, 10 U/ml penicillin, and 10 μg/ml streptomycin for 48 hours at 37° C. before use. Cells of each well are labeled by incubation with [³H]-arachidonic acid (Amersham Corp., 210 Ci/mmol) at 0.5 μCi/ml in 1 ml MEM supplemented with 10 mM HEPES, pH 7.5, and 0.5% fatty-acid-free bovine serum albumin for 2 hours at 37° C. The cells are then washed twice with 1 ml of the same buffer.

Candidate modulator compounds are added in 1 ml of the same buffer, either alone or with 10 μM ATP, and the cells are incubated at 37° C. for 30 minutes. Buffer alone and mock-transfected cells are used as controls. Samples (0.5 ml) from each well are counted by liquid scintillation spectroscopy. Agonists which activate the receptor will lead to potentiation of the ATP-stimulated release of [³H]-arachidonic acid. This potentiation is blocked by antagonists.

Extracellular Acidification Rate

In yet another assay, the effects of candidate modulators of DmGPCR activity are assayed by monitoring extracellular changes in pH induced by the test compounds. See, e.g., Dunlop et al., J. Pharmacological and Toxicological Methods, 1998, 40(1), 47-55. In one embodiment, CHO cells transfected with a DmGPCR expression vector are seeded into 12 mm capsule cups (Molecular Devices Corp.) at 4×10⁵ cells/cup in MEM supplemented with 10% fetal bovine serum, 2 mM L-glutamine, 10 U/ml penicillin, and 10 μg/ml streptomycin. The cells are incubated in this medium at 37° C. in 5% CO₂ for 24 hours.

Extracellular acidification rates are measured using a Cytosensor microphysiometer (Molecular Devices Corp.). The capsule cups are loaded into the sensor chambers of the microphysiometer and the chambers are perfused with running buffer (bicarbonate-free MEM supplemented with 4 mM L-glutamine, 10 units/ml penicillin, 10 μg/ml streptomycin, 26 mM NaCl) at a flow rate of 100 μl/minute. Candidate agonists or other agents are diluted into the running buffer and perfused through a second fluid path. During each 60-second pump cycle, the pump is run for 38 seconds and is off for the remaining 22 seconds. The pH of the running buffer in the sensor chamber is recorded during the cycle from 43-58 seconds, and the pump is re-started at 60 seconds to start the next cycle. The rate of acidification of the running buffer during the recording time is calculated by the Cytosoft program. Changes in the rate of acidification are calculated by subtracting the baseline value (the average of 4 rate measurements immediately before addition of a modulator candidate) from the highest rate measurement obtained after addition of a modulator candidate. The selected instrument detects 61 mV/pH unit. Modulators that act as agonists of the receptor result in an increase in the rate of extracellular acidification compared to the rate in the absence of agonist. This response is blocked by modulators which act as antagonists of the receptor.

### Example 9

### Matching DmGPCRs with Peptide Ligands

Cell Cultures and transfections

Wild type Chinese hamster ovary (CHO-KI) cells (from the American Type Culture Collection, Rockville, Md.) or CHO-10001A cells were cultured at 37° C. in a humidified atmosphere of 5% CO₂ in air in DMEM media supplemented with 10% heat-inactivated FBS, 10 μg/ml gentamicin, 0.1 mM nonessential amino acids to give complete DMEM media. Cells were transfected with orphan GPCR DNAs in the pCR3.1 vector, using LipofectAMINE PLUS™, essentially according to the manufacturer's instructions. Briefly, CHO cells were plated on 10 cm sterile tissue culture dishes (Coming Glass Works, Corning, N.Y.), and they were about 50-60% confluent the day of transfection. In a plastic tube, PLUS (20 μl/plate) was added to cDNA plasmid (5 μg/plate) which was earlier diluted into 0.75 ml OptiMEM, mixed and incubated at room temp for 15 min. Separately, LipofectAMINE (30 μl/plate) was mixed with 0.75 ml OptiMEM and added to the pre-complexed DNA/PLUS mixture and incubated at room temp. for 15 minutes. Medium on the cells was replaced with serum-free transfection medium (plain DMEM, 5 ml/plate), and the DNA-PLUS-LipofectAMINE complex was added (1.5 ml per plate) and mixed gently into the medium followed by a 3 hr incubation at 37° C./5% CO₂. Then the medium was supplemented with the complete DMEM medium containing 20% FBS (6.5 ml ml/plate) and the incubation continued at 37° C./5% CO₂ for 24 to 48 hrs. A plasmid for Green Fluorescent Protein (GFP, 4 μg/plate) was used for transient GFP expression in CHO cells to estimate the transfection yields under the same conditions also used for GPCRs.

Membrane Preparation

The transfected cells were washed once with ice-cold Dulbecco's phosphate buffered saline (PBS), 5 ml per 10 cm plate, and scraped into 5 ml of the same buffer. Cell suspensions from multiple plates were combined and centrifuged at 500×g for 10 min at 4° C. The cell pellet was reconstituted in ice-cold TEE (25 mM TRIS, 5 mM EGTA, 5 mM EDTA). Convenient aliquots were snap-frozen in liquid nitrogen and stored at −70° C. After thawing, the cells were homogenized and centrifuged at 4° C., 500×g for 5 minutes to pellet nuclei and unbroken cells. The supernatant was centrifuged at 47,000×g for 30 minutes at 4° C. The membrane pellet was washed once with TEE, resuspended in 20 mM HEPES, pH 7.4, 100 mM NaCl, 10 mM MgCl₂, 1 mM EDTA (assay buffer), aliquoted and frozen in liquid nitrogen. Membrane aliquots were stored at 70° C. Membrane protein concentration was determined using the BCA Protein Assay Reagent from Pierce (Rockford, Ill.) and BSA as standard.

[³⁵S]GTPγS Binding Assay

Aliquots of cell membranes were thawed, homogenized, and diluted into buffer containing 20 mM HEPES, pH 7.4, 100 mM NaCl, 10 mM MgCl₂, 1 mM EDTA (assay buffer). Initially, reaction mixtures were prepared in 96-well polypropylene plates (Nunc). In each well, peptide aqueous solution (20 μl, 10×), or water controls (20 μl), 18.2 μM GDP in assay buffer (0.11 ml, 10 μM final), and membranes suspended in assay buffer (50 μl, 10 μg membrane protein) were mixed and placed on ice. The ligand-GDP-membrane mixtures were incubated for 20 min. at room temperature on a shaking platform and then placed on ice. To each sample, 20 μl guanosine-5′-O-(3-[³⁵S]thio)-triphosphate ([³⁵S]GTPγS) (600-1,200 Ci/mmol from New England Nuclear, Boston, Mass.) was added to ˜40,000 cpm/0.2 ml, or a final concentration of 0.1 nM. Plates with the incubation mixtures (0.2 ml/well total) were incubated at room temperature for 45 minutes. Reaction mixture aliquots, 0.175 ml each, were then transferred into wash buffer pretreated (100 μl/well) 96-well FB MultiScreen filter plates (Millipore) and vacuum filtered using a MultiScreen Vacuum manifold (Millipore). Then the membranes were washed 3 times with 0.25 ml ice-cold wash buffer/well (10 mM HEPES, 10 mM MgCl₂, pH 7.4) and vacuum filtered. After the last wash, Supermix Opti-phase scintillation fluid (25 μl/well, Wallac) was added and the plates were scaled and counted in a Trilux 1450 Microbeta counter (Wallac) for 1 minute/well. As positive controls, membranes from CHO cells stably expressing a dopamine type 2 (rD₂) receptor were treated with 1 mM dopamine in 0.025% ascorbic acid (100 μM dopamine final) or vehicle (0.0025% ascorbic acid final). Non-specific binding was measured in the presence of 100 μM cold GTPγS and was subtracted from the total. Each treatment was carried out in triplicates.

Data Analysis

Ligand-induced stimulation of [³⁵S]GTPγS binding was expressed as fold increase over the basal activity with no ligand added. Each treatment was run either in triplicate, or, on occasion, in duplicate, and the binding (cpm) was calculated as means +/−standard deviations. Dose-response curves for the receptor/ligand systems were analyzed using a non-linear least square SAS model, y=BₘₐₓX/(K_{d}+X). Other dose-response curves were analyzed using Prism (GraphPad Software, Inc. San Diego, Calif.) and the following equation y=Bottom+(Top−Bottom)/(1+10^{LogEC50-X}).

Results

Originally, we have chosen the GTPγS assay as a functional assay because agonist-driven stimulation of GTPγS reflects early events in the DmGPCR activation cascade, regardless of further activation pathways of various down-stream signaling events. This appears especially useful for the assessment of possible activation of orphan DmGPCRs with unknown functions and unknown signaling pathways. The GTPγS assay was carried out with membranes prepared from CHO cells transiently transfected with DNA encoding Drosophila GPCRs using a 96-well MultiScreen G/IFB filter plates and a MultiScreen vacuum manifold (Millipore) for filtration. Since the GTPγS assay is known to poorly recognize GPCRs coupled to the Gq class of G-proteins, a Ca⁺² mobilization assay based on a FLIPR readout was used as well to evaluate Gq-coupled orphan GPCRs in CHO cells transiently transfected with DNA encoding Drosophila GPCRs.

Using GTPγS assay, DmGPCR1 (PnuFlyPep34651) was found to be activated by two Drosophila NPF-like peptides, AQRSPSLRLRF-NH₂ (SEQ ID NO: 186), and PIRSPSLRLRF-NH₂ (SEQ ID NO: 187) as reflected in the determined EC₅₀ values of about 2.5 nM. Activation with DPKQDFMRF-NH₂ (SEQ ID NO: 26) and PDNFMRF-NH₂ (SEQ ID NO: 27) resulted in the GTPγS responses with EC₅₀'s ranging from 370 nM to 500 nM. As reported by Nambu et al. (Neuron, 1988, 1, 55-61), these two peptides are encoded on the same precursor gene together with nine other FaRPs. Additional FaRPs and other neuropeptides which also stimulated GTPγS binding, although less effectively (EC₅₀'s in the range of 5 to 10 μM), included the following peptides: TDVDHVFLRF-NH₂ (SEQ ID NO: 25), TPAEDFMRF-NH₂ (SEQ ID NO: 28), SLKQDFMHF-NH₂ (SEQ ID NO: 29), SVKQDFMHF-NH₂ (SEQ ID NO: 30), AAMDRY-NH₂ (SEQ ID NO: 31), and SVQDNFMHF-NH₂ (SEQ ID NO: 32). In addition, the FLIPR assay identified a Colorado potato beetle peptide, ARGPQLRLRF-NH₂ (SEQ ID NO: 33), matched to DmGPCR1 receptor with an EC₅₀ of 100-200 nM. Our data indicate that Dmgpcr1 should be classified as a short neuropeptide F receptor since it is strongly activated by the two short NPF peptides, SEQ ID NO: 186 and SEQ ID NO: 187.

As shown by the GTPγS responses, DmGPCR4 (PnuFlyPep 67393) was activated by a Drosophila melanogaster allatostatin, drostatin-3 (SRPYSFGL-NH₂ (SEQ ID NO: 165)) with an EC₅₀ in the low nanomolar range, as well as by various Diplotera punctata (cockroach) allatostatins, namely: GDGRLYAFGL-NH₂ (SEQ ID NO: 34), DRLYSFGL-NH₂ (SEQ ID NO: 35), APSGAQRLYGFGL-NH₂ (SEQ ID NO: 36), and GGSLYSFGL-NH₂ (SEQ ID NO: 37) (EC₅₀'s in the range of ca. 20-280 nM). The same peptides elicited a very strong calcium signal when tested at 10 μM by FLIPR. DmGPCR4 has been recently cloned by Lenz et al., supra, and classified as a second putative allatostatin receptor (DAR11). However, no pharmacological data on receptor activation have been reported to date. To our knowledge this is the very first experimental evidence that various allatostatins do activate this receptor.

As shown by the GTPγS responses, DmGPCR5 (GenBank Accession No. AX128628) when transiently expressed in CHO-10001A cells, was activated by drotachykinins (DTKs), namely DTK-1 (APTSSFIGMR-NH₂) (SEQ ID NO: 169), Met8-DTK-2 (APLAFYGMR-NH₂) (SEQ ID NO: 170), DTK-2 (APLAFYGLR-NH₂) (SEQ ID NO: 171, DTK-3 (APTGFTGMR-NH₂) (SEQ ID NO: 172), DTK-4 (APVNSFVGMR-NH₂) (SEQ ID NO: 173), and DTK-5 (APNGFLGMR-NH₂) (SEQ ID NO: 174). In a dose-response experiment, DTK-1, Met8-DTK-2, DTK-3, and DTK-5 stimulated GTPγS binding with EC₅₀'s in the 250-500 nM range and the maximal stimulation ca. 1.5-fold above basal level. DTK-2 and DTK-4 were less potent as judged by their EC50's in the low micromolar range. In the calcium mobilization assay (FLIPR), DmGPCR5 showed Ca⁺² responses to the same DTKs with the EC₅₀'s in the 1-20 nM range. Additionally, DTK-5, DTK-2 and Met8-DTK-2 were tested in a cAMP (reporter-gene-based) assay and stimulated cAMP release in a dose-response fashion with EC₅₀'s of 197 nM, 1.06 μM, and 583 nM, respectively. These data indicate that DmGPCR5 couples to both Gs (cAMP) and Gq (Ca⁺²)-mediated signaling pathways which is analogous to the signaling pathways reported for vertebrate tachykinin receptors.

DmGPCR6a (M811490) was reported as a PYY receptor by Li et al. (J. Biol. Chem., 1992, 267, 9-12). Using the GTPγS assay, the peptides listed in Table 7, tested at 5 μM, stimulated GTPγS binding (1.7 to 4 fold increase above the basal) to membranes from CHO cells transfected with a DNA encoding DmGPCR6a. It is noteworthy that, in addition to a battery of insect and C. elegans peptides that activated this receptor, also human NPFF (FLFQPQRF-NH₂ (SEQ ID NO: 59)) was found to be a ligand for DmGPCR6 (4-fold increase in GTPγS binding by 5 μM NPFF).

Dmgpcr6aL and Dmgpcr6bL are two splice variants of DmGPCR6a (M811490). The latter was reported as a PYY receptor by Li et al. (J. Biol. Chem., 1992, 267, 9-12). We name both DmGPCR6aL and DmGPCR6bL, RF-amide receptors since they recognize only peptides that have an Arg-Phe-NH₂ (RFa) sequence at the C-terminus. The peptides that these DmGPCRs did not “see” have different than RFa sequences at the C-end (e.g., SFa, QFa, YFa, RLa, DWa, RPa, HFa, LQa, SNa etc.). In the calcium mobilization assay (FLIPR), Dmgpcr6aL and Dmgpcr6bL showed very strong Ca⁺² responses to a battery of FaRPs tested at 10 μM. The sequences shown below in Table 7 represent all the identified active FaRPs belonging to various species including Drosophila, C. elegans, A. suum, Mollusca, P. redivivus, Trematoda, lobster, human, and leech: The only exception to the C-end “RFamide rule” was the peptide pGluDRDYRPLQF-NH₂ (SEQ ID NO: 120), whose C-terminus ends with an Gln-Phe-NH₂ (QFa) sequence. Interestingly, both Dmgpcr6aL and Dmgpcr6bL also recognized NPFF (FLFQPQRF-NH₂ (SEQ ID NO: 152)), a mammalian peptide with the RFamide sequence at the C-terminus. (Note in the results above that p-Glu or pQ refers to pyroglutamic acid.)

As shown by FLIPR analysis, DmGPCR7 (GenBank Accession No. AX128636) transiently expressed in CHO-10001A cells, was activated by the leucokinins (LKs) and related peptides, namely LK-I (DPAFNSWGa) (SEQ ID NO: 175), LK-V (GSGFSSWGa) (SEQ ID NO: 176), LK-VI (pGlu-SSFHSWGa) (SEQ ID NO: 177), LK-VIII (GSAFYSWGa) (SEQ ID NO: 178), Culekinin (NPFHSWGa) (SEQ ID NO: 179), mollusc lymnokinin (PSFHSWSa) (SEQ ID NO: 180), and Drosophila leucokinin-like peptides DLK-1 (NSVVLGKKQRFHSWGa) (SEQ ID NO: 181), DLK-2 (pGlu-RFHSWGa) (SEQ ID NO: 182) and DLK-2A (QRFHSWGa) (SEQ ID NO: 183). DmGPCR7 was best activated by the LK peptides having a common C-terminal tetrapeptide sequence, HSWGa. Treatments with this group of peptides, which included DLK-1, DLK-2, DLK-2a, LK-VI and Culekinin, resulted in a very potent intracellular calcium release (EC₅₀'s in the picomoloar to subnanomolar range). In contrast other locust LK's with the C-terminal S/NSWGa (LK-I, LK-V) as well the Lymnaea LK (SEQ ID NO: 180), showed lower potency (EC₅₀'s 15-30 nM) and the LK-VIII with its YSWGa C-terminal sequence was the least potent in the series (EC₅₀'s in the 100-200 nM range). No GTPγS responses to these peptides could be detected in membranes prepared from DmGPCR7/CHO cells, which is indicative of a Gq/1-coupled receptor. Therefore, DmGPCR7 was identified as a calcium-signaling leucokinin receptor (most likely G_{q/11}-coupled) and matched with the drolucokinins as its cognate ligands.

As shown by the GTPγS responses, DmGPCR8 (GenBank Accession No. AX128638) transiently expressed in CHO-10001A cells was activated by the Manduca sexta allatostatin-C (AST-C, or Manse-AC), (pGlu-VRFRQCYFNPISCF-OH) (SEQ ID NO: 184) or drostatin-C (DST-C), also called flatline peptide (FLT) (pGlu-VRYRQCYFNPISCF-OH) (SEQ ID NO: 185). In a dose response GTPγS-binding experiment, a high potency AST-C and DST-C responses were detected (EC₅₀ 's in a low nanomolar range). These activities were completely abolished by cell pretreatment with pertussin toxin indicating Gi/Go involvement in receptor activation. In a direct calcium mobilization assay (FLIPR), DmGPCR8 did not show any activity when challenged with AST-C or DST-C. However, strong calcium releasing activity to DST-C was detected in CHO-10001A cells co-transfected with DmGPCR8 and chimeric G-proteins Gqi5 or Gqo5 (EC₅₀'s ca 30 nM). On the other hand, coupling to Gqz5 was less efficient (EC₅₀ 244 nM) and no calcium mobilization was observed in cells co-transfected with DmGPCR8 and Gqs5. These results indicate that DmGPCR8 is an inhibitory receptor in CHO cells that preferably couples to the Gi/Go type G-proteins. The presented results unequivocally identify DmGPCR8 as a DST-C/FLT receptor.

DmGPCR9 has been matched with FDDY(SO₃H)GHLRF-NH₂ (SEQ ID NO:157), based on its very strong signal in the calcium mobilization assay (EC₅₀ in the low nanomolar range). The fact that no GTPγS responses to this peptide were detected with membranes prepared from CHO cells transfected with a DNA encoding DmGPCR9, indicates that DmGPCR9 is most likely coupled to Gq signaling pathways. FDDY(SO₃H)GHLRF-NH₂ (SEQ ID NO: 157) represents a Met7-Leu7 analog of the naturally occurring drosulfakinin-1 (DSK-1), FDDY(SO₃H)GHMRF-NH₂ (SEQ ID NO: 159). Therefore we assign the DmGPCR9 receptor as a sulfakinin receptor. This match is very specific since even FDDYGHLRF-NH₂ (SEQ ID NO:158), which is an unsulfated counterpart of FDDY(SO₃H)GHLRF-NH₂ (SEQ ID NO: 157), showed only a very weak calcium signal when tested at 10 μM and none of the other 117 tested FaRPs and related peptides showed any activity either in FLIPR or in the GTPγS assay at the DmGPCR9 receptor.

A table matching the ligands with their associated receptors is shown below in Table 7.

TABLE 7

| GPCR | SEQ ID NO | Peptide Matching Sequence |
|---|---|---|
| dmgpcr1 | SEQ ID NO:186 | AQRSPSLRLRF-NH₂ |
| | SEQ ID NO:187 | PIRSPSLRLRF-NH₂ |
| | SEQ ID NO:25 | TDVDHVFLRF-NH₂ |
| | SEQ ID NO:26 | DPKQDFMRF-NH₂ |
| | SEQ ID NO:27 | PDNFMRF-NH₂ |
| | SEQ ID NO:28 | TPAEDFMRF-NH₂ |
| | SEQ ID NO:29 | SLKQDFMHF-NH₂ |
| | SEQ ID NO:30 | SVKQDFMHF-NH₂ |
| | SEQ ID NO:31 | AAMDRY-NH₂ |
| | SEQ ID NO:32 | SVQDNFMHF-NH₂ |
| | SEQ ID NO:33 | ARGPQLRLRF-NH₂ |
| dmgpcr4 | SEQ ID NO:34 | GDGRLYAFGL-NH₂ |
| | SEQ ID NO:35 | DRLYSFGL-NH₂ |
| | SEQ ID NO:36 | APSGAQRLYGFGL-NH₂ |
| | SEQ ID NO:37 | GGSLYSFGL-NH₂ |
| dmgpcr6 | SEQ ID NO:38 | FIRF-NH₂ |
| (6a) | SEQ ID NO:39 | KNEFIRF-NH₂ |
| | SEQ ID NO:40 | FMRF-NH₂ |
| | SEQ ID NO:41 | KSAFMRF-NH₂ |
| | SEQ ID NO:42 | KPNFLRF-NH₂ |
| | SEQ ID NO:43 | FLRF-NH₂ |
| | SEQ ID NO:44 | YLRF-NH₂ |
| | SEQ ID NO:45 | KPNFLRY-NH₂ |
| | SEQ ID NO:46 | TNRNFLRF-NH₂ |
| | SEQ ID NO:47 | RNKFEFIRF-NH₂ |
| | SEQ ID NO:48 | AGPRFIRF-NH₂ |
| | SEQ ID NO:49 | GLGPRPLRF-NH₂ |
| | SEQ ID NO:50 | IL-Nle-RF-NH₂ |
| | SEQ ID NO:51 | AGAKFIRF-NH₂ |
| | SEQ ID NO:52 | APKPKFIRF-NH₂ |
| | SEQ ID NO:53 | KSAFVLRF-NH₂ |
| | SEQ ID NO:54 | TKFQDFLRF-NH₂ |
| | SEQ ID NO:55 | SAEPFGTMRF-NH₂ |
| | SEQ ID NO:56 | ASEDALFGTMRF-NH₂ |
| | SEQ ID NO:57 | SADDSAPFGTMRF-NH₂ |
| | SEQ ID NO:58 | EDGNAPFGTMRF-NH₂ |
| | SEQ ID NO:59 | FLFQPQRF-NH₂ |
| dmgpcr6 | SEQ ID NO:60 | SADPNFLRF-NH₂ |
| 6aL and | SEQ ID NO:61 | SQPNFLRF-NH₂ |
| 6bL | SEQ ID NO:62 | ASGDPNFLRF-NH₂ |
| | SEQ ID NO:63 | SDPNFLRF-NH₂ |
| | SEQ ID NO:64 | AAADPNFLRF-NH₂ |
| | SEQ ID NO:65 | PNFLRF-NH₂ |
| | SEQ ID NO:66 | KPNFLRF-NH₂ |
| | SEQ ID NO:67 | AGSDPNFLRF-NH₂ |
| | SEQ ID NO:68 | KPNFLRY-NH₂ |
| | SEQ ID NO:69 | SPREPIRF-NH₂ |
| | SEQ ID NO:70 | LRGEPIRE-NH₂ |
| | SEQ ID NO:71 | SPLGTMRF-NH₂ |
| | SEQ ID NO:72 | EAEEPLGTMRF-NH₂ |
| | SEQ ID NO:73 | ASEDALFGTMRF-NH₂ |
| | SEQ ID NO:74 | EDGNAPFGTMRF-NH₂ |
| | SEQ ID NO:75 | SAEPFGTMRF-NH₂ |
| | SEQ ID NO:76 | SADDSAPFGTMRF-NH₂ |
| | SEQ ID NO:77 | KPTFIRF-NH₂ |
| | SEQ ID NO:78 | ASPSFIRF-NH₂ |
| | SEQ ID NO:79 | GAKFIRF-NH₂ |
| | SEQ ID NO:80 | AGAKFIRF-NH₂ |
| | SEQ ID NO:81 | APKPKFIRF-NH₂ |
| | SEQ ID NO:82 | KSAYMRF-NH₂ |
| | SEQ ID NO:83 | SPMQRSSMVRF-NH₂ |
| | SEQ ID NO:84 | SPMERSAMVRE-NH₂ |
| | SEQ ID NO:85 | SPMDRSKMVRF-NH₂ |
| | SEQ ID NO:86 | KNEFIRF-NH₂ |
| | SEQ ID NO:87 | KPSFVRF-NH₂ |
| | SEQ ID NO:88 | pQPKARSGYIRF-NH₂ |
| | SEQ ID NO:89 | AMRNALVRF-NH₂ |
| | SEQ ID NO:90 | ASGGMRNALVRF-NH₂ |
| | SEQ ID NO:91 | NGAPQPFVRF-NH₂ |
| | SEQ ID NO:92 | RNKFEFIRF-NH₂ |
| | SEQ ID NO:93 | SDRPTRAMDSPLIRF-NH₂ |
| | SEQ ID NO:94 | AADGAPLIRF-NH₂ |
| | SEQ ID NO:95 | APEASPFIRF-NH₂ |
| | SEQ ID NO:96 | ASPSAPLIRF-NH₂ |
| | SEQ ID NO:97 | SPSAVPLIRF-NH₂ |
| | SEQ ID NO:98 | ASSAPLIRF-NH₂ |
| | SEQ ID NO:99 | KHEYLRE-NH₂ |
| | SEQ ID NO:100 | SLLDYRF-NH₂ |
| | SEQ ID NO:101 | EIVFHQISPIFFRF-NH₂ |
| | SEQ ID NO:102 | GGPQGPLRF-NH₂ |
| | SEQ ID NO:103 | GPSGPLRF-NH₂ |
| | SEQ ID NO:104 | AQTFVRF-NH₂ |
| | SEQ ID NO:105 | GQTFVRF-NH₂ |
| | SEQ ID NO:106 | KSAFVRF-NH₂ |
| | SEQ ID NO:107 | KSQYIRF-NH₂ |
| | SEQ ID NO:108 | DVPGVLRF-NH₂ |
| | SEQ ID NO:109 | KSVPGVLRF-NH₂ |
| | SEQ ID NO:110 | SEVPGVLRF-NH₂ |
| | SEQ ID NO:111 | SVPGVLRF-NH₂ |
| | SEQ ID NO:112 | DFDGAMPGVLRF-NH₂ |
| | SEQ ID NO:113 | EIPGVLRF-NH₂ |
| | SEQ ID NO:114 | WANQVRF-NH₂ |
| | SEQ ID NO:115 | ASWASSVRF-NH₂ |
| | SEQ ID NO:116 | AMMRF-NH₂ |
| | SEQ ID NO:117 | GLGPRPLRE-NH₂ |
| | SEQ ID NO:118 | SPSAKWMRF-NH₂ |
| | SEQ ID NO:119 | TKFQDFLRF-NH₂ |
| | SEQ ID NO:120 | pQDRDYRPLQF-NH₂ |
| | SEQ ID NO:121 | FIRF-NH₂ |
| | SEQ ID NO:122 | AVPGVLRF-NH₂ |
| | SEQ ID NO:123 | GDVPGVLRF-NH₂ |
| | SEQ ID NO:124 | SDIGISEPNFLRF-NH₂ |
| | SEQ ID NO:125 | SGKPTFIRF-NH₂ |
| | SEQ ID NO:126 | AEGLSSPLIRF-NH₂ |
| | SEQ ID NO:127 | FDRDFMRF-NH₂ |
| | SEQ ID NO:128 | AGPRFIRF-NH₂ |
| | SEQ ID NO:129 | GMPGVLRF-NH₂ |
| | SEQ ID NO:130 | IL-Nle-RF-NH₂ |
| | SEQ ID NO:131 | LQPNFLRF-NH₂ |
| | SEQ ID NO:132 | KPNFIRF-NH₂ |
| | SEQ ID NO:133 | FMRF-NH₂ |
| | SEQ ID NO:134 | FLRF-NH₂ |
| | SEQ ID NO:135 | YIRF-NH₂ |
| | SEQ ID NO:136 | GNSFLRF-NH₂ |
| | SEQ ID NO:137 | DPSFLRF-NH₂ |
| | SEQ ID NO:138 | pQDFMRF-NH₂ |
| | SEQ ID NO:139 | KPNQDFMRF-NH₂ |
| | SEQ ID NO:140 | TDVDHVFLRF-NH₂ |
| | SEQ ID NO:141 | AAMDRY-NH₂ |
| | SEQ ID NO:142 | SPKQDFMRF-NH₂ |
| | SEQ ID NO:143 | PDNFMRF-NH₂ |
| | SEQ ID NO:144 | DPKQDFMRF-NH₂ |
| | SEQ ID NO:145 | TPAEDFMRE-NH₂ |
| | SEQ ID NO:146 | SDNFMRF-NH₂ |
| | SEQ ID NO:147 | YLRF-NH₂ |
| | SEQ ID NO:148 | SDRNFLRF-NH₂ |
| | SEQ ID NO:149 | TNRNFLRF-NH₂ |
| | SEQ ID NO:150 | PDVDHVFLRF-NH₂ |
| | SEQ ID NO:151 | pQDVDHVFLRF-NH₂ |
| | SEQ ID NO:152 | FLFQPQRF-NH₂ |
| | SEQ ID NO:153 | ARGPQLRLRF-NH₂ |
| | SEQ ID NO:154 | FDDY(SO₃H)GHLRF-NH₂ |
| | SEQ ID NO:155 | FDDYGHLRF-NH₂ |
| | SEQ ID NO:156 | MDSNFIRF-NH₂ |
| dmgpcr9 | SEQ ID NO:157 | FDDY(SO₃H)GHLRF-NH₂ |
| dmgpcr5 | SEQ ID NO:169 | APTSSFIGMR-NH₂ |
| | SEQ ID NO:170 | APLAFYGMR-NH₂ |
| | SEQ ID NO:171 | APLAFYGLR-NH₂ |
| | SEQ ID NO:172 | APTGFTGMR-NH₂ |
| | SEQ ID NO:173 | APVNSFVGMR-NH₂ |
| | SEQ ID NO:174 | APNGFLGMR-NH₂ |
| dmgpcr7 | SEQ ID NO:175 | DPAFNSWG-NH₂ |
| | SEQ ID NO:176 | GSGFSSWG-NH₂ |
| | SEQ ID NO:177 | pGlu-SSFHSWG-NH₂ |
| | SEQ ID NO:178 | GASFYSWG-NH₂ |
| | SEQ ID NO:179 | NPFHSWG-NH₂ |
| | SEQ ID NO:180 | PSFHSWS-NH₂ |
| | SEQ ID NO:181 | NSVVLGKKQRFHSWG-NH₂ |
| | SEQ ID NO:182 | pGlu-RFHSWG-NH₂ |
| | SEQ ID NO:183 | QRFHSWG-NH₂ |
| dmgpcr8 | SEQ ID NO:184 | 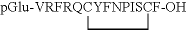 |
| | SEQ ID NO:185 | 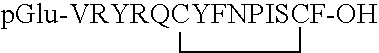 |

### Example 10

### Competition Assay

Preparation Of Mono-Iodinated Peptide

The peptide is iodinated via a typical chloramine T procedure. Added to a 2 ml glass vial are 10 μl of a 1 mM water solution of peptide, 10 μL of 0.1M (pH 7.99) sodium phosphate buffer, 1.0 mCi [¹²⁵I] sodium iodide, and 5 μl of a 2 mg/ml chloramine T solution (in the phosphate buffer). The mixture is vortexed for 60 seconds and the reaction stopped by the addition of 25 μl of a 5 mg/ml solution of sodium metabisulfite in phosphate buffer. The mixture then undergoes HPLC by injecting it onto a Vydac C18 (0.45×15 cm) column and subjecting it to gradient separation. The gradient used is 70% A and 30% B at time zero to 20% A and 80% B at time 25 minutes (A=0.1M NH4 acetate in water. B=0.1M NH4 acetate in water 40%: CH₃CN 60%, v:v.). Flow rate is 1.0 ml/minute. Samples are collected into 0.25 ml capture buffer (0.1M sodium phosphate buffer with 0.5% bovine serum albumin, 0.1% Triton X10O and 0.05% Tween 20) at 30 second intervals from t=8 to t=20 minutes. Monoiodo peptide typically elutes at t=11 minutes and the yield is approximately 100 μCi in 0.75 ml.

Binding Assay

96-well plates used are Millipore Multiscreen® filtration plates (FB opaque 1.0 μM glass fiber type B, cat. # MAFBNOB50). A Millipore Multiscreen® solvent resistant manifold (cat. # MAVMO960R) is used in conjunction with the plates to filter the assay at termination. Each replicate is one well and has a volume of 100 ul containing 5 ug protein (preparation described above). Each test group contains two replicates. For each test compound, one group is run with [¹²⁵I]peptide only (for total binding) and one with 1 μM (or as designated) concentration of the test compound and [¹²⁵I]peptide (for non-specific binding). The order of adding reagents for each replicate is: assay buffer (20 mM HEPES, 10 mM MgCl₂, 1% bovine serum albumin, pH 7.4) test compound (made up in assay buffer), [¹²⁵I]peptide (in assay buffer) and membrane suspension (in assay buffer). The addition of the membrane suspension initiates the binding reaction which is run for 30 minutes at room temperature (22° C.). Following the 30 minute incubation each plate is place on the filtration manifold and vacuum is applied, pulling the liquid through the filter (discarded) and catching the protein on the filters in each well. For washing, the vacuum is released and 200 μl assay buffer is added to each well followed by reapplication of the vacuum. This washing is repeated twice more (total of 3×washes for each replicate). Following washing, the plastic covering on the underside of each plate is removed and the plate placed in a bottom sealed Microbeta® scintillation counting cassette (cat # 1450-105). 25 μl of scintillant is added to each well and the plate is placed on a rotary shaker at 80 rpm for one hour and then allowed to sit overnight. The following day the plate is counted in a Microbeta® scintillation counter. The mean non-specific binding is subtracted from the mean total binding to yield specific binding for both the standard (peptideamide) and the unknowns.

As those skilled in the art will appreciate, numerous changes and modifications may be made to the embodiments of the invention described above without departing from the spirit of the invention. It is intended that all such variations fall within the scope of the invention.

The entire disclosure of each publication cited herein is hereby incorporated by reference.

What is claimed is:

1. A method for identifying a modulator of binding and/or function between a DmGPCR1 and a DmGPCR1 binding partner, comprising the steps of:
(a) contacting a DmGPCR1 binding partner and a composition comprising a DmGPCR1 in the presence or in the absence of a putative modulator compound;
(b) detecting binding between the DmGPCR1 binding partner and the DmGPCR1; and
(c) determining whether binding or function in the presence of said putative modulator compound is increased or decreased compared to binding or function in the absence of said putative modulator compound,
wherein said DmGPCR1 binding partner has a sequence with at least 70% sequence identity to a sequence selected from the group consisting of SEQ ID NO: 186 and SEQ ID NO: 187.

2. The method according to claim 1, wherein said DmGPCR1 binding partner has a sequence with at least 80% sequence identity to a sequence selected from the group consisting of SEQ ID NO: 186 and SEQ ID NO: 187.

3. The method according to claim 1, wherein said DmGPCR1 binding partner has a sequence with at least 95% sequence identity to a sequence selected from the group consisting of SEQ ID NO: 186 and SEQ ID NO: 187.

4. The method according to claim 1, wherein said DmGPCR1 binding partner has a sequence selected from the group consisting of SEQ ID NO: 186 and SEQ ID NO: 187.

5. A method of controlling a population of insects comprising administering a binding partner or a modulator of a DmGPCR1 polynucleotide or polypeptide to an insect to modify the expression or activity of the DmGPCR1, wherein said binding partner has a sequence with at least 70% seqeunce identity to a sequence selected from the group consisting of SEQ ID NO: 186 and SEQ ID NO: 187.

6. The method according to claim 5, wherein said binding partner has a sequence with at least 80% identity to a sequence selected from the group consisting of SEQ ID NO: 186 and SEQ ID NO: 187.

7. The method according to claim 5, wherein said binding partner has a sequence with at least 95% identity to a sequence selected from the group consisting of SEQ ID NO: 186 and SEQ ID NO: 187.

8. The method according to claim 5, wherein said binding partner has a sequence selected from the group consisting of SEQ ID NO: 186 and SEQ ID NO: 187.

9. The method according to claim 5, wherein said insect is selected from the group consisting of a fly, a fruitfly, a tick, a flea, lice, a mite, and a cockroach.

10. A method of treating or preventing a disease or condition caused by an ectoparasite in a subject comprising administering to said subject a therapeutically effective amount of a DmGPCR1 binding partner, wherein said DmGPCR1 binding partner has a sequence with at least 70% identity to a sequence selected from the group consisting of SEQ ID NO: 186 and SEQ ID NO: 187.

11. The method according to claim 10, wherein said DmGPCR1 binding partner has a sequence with at least 80% identity to a sequence selected from the group consisting of SEQ ID NO: 186 and SEQ ID NO: 187.

12. The method according to claim 10, wherein said DmGPCR1 binding partner has a sequence with at least 95% identity to a sequence selected from the group consisting of SEQ ID NO: 186 and SEQ ID NO: 187.

13. The method according to claim 10, wherein said DmGPCR1 binding partner has a sequence selected from the group consisting of SEQ ID NO: 186 and SEQ ID NO: 187.

14. The method according to claim 10, wherein said subject is a human.

15. The method according to claim 10, wherein said disease is selected from the group consisting of Alzheimer's disease, Parkison's disease, Huntington's Disease, neuromsuscular diseases, and neurodegenerative dieseases.
